SEQUENCE LISTING (3 sequences)

SEQ ID NO: 1 - protein, 460 aa, Artificial Sequence
  A4-Fla2, also known as Fla2=Synthetic Construct
MVVQHNLRAMNSNRMLGITQGSLNKSTEKLSSGYKVNRAADDAAGLSISEKMRKQIRGLSQASLNAEDGISAVQTAEGALTEVHDMLQRMNELAVKAANGTNSTSDRQTIQDEVDQLLTEIDRVAETTKFNELYTLKGDEDKVTRYLSAHDAGIEGTLTQGATNATFSMDQLKFGDTIMIAGREYHISGTQKQQGEIITSSVKIGQQVTIDGIMYTCTATVSNADKFELTKDDLIAKLDTSSLSIMSVNGKTYYGAGITDDRTVVSSIGAYKLIQKELGLASSIGADGSTQASVNAGVDGKTLKKPSFEGKWVFSIDKGSVQVREDIDFSLHVGADADMNNKIAVKIGALDTKGLGIQGLNVKDTTGAAATYAIDSIADAVARISAQRSLLGAVQNRLEHTINNLDNVVENTTAAESQIRDTDMATEMVKYSNNNVLAQAGQSMLAQSNQANQGVLSLLG

SEQ ID NO: 2 - protein, 493 aa, Unknown
  Description of Unknown Sequencefull-length CBir-1 flagellin from C3H/HeJ Bir mouse cecal bacteria genomic DNA random shear expression library PCR amplification
MVVQHNLQAMNSNRMLGITQKTASKSTEKLSSGYAINRAADNAAGLAISEKMRKQIRGLTQASTNAEDGISSVQTAEGALTEVHDMLQRMNELAIQAANGTNSEDDRSYIQDEIDQLTQEIDRVAETTKFNETYLLKGDTKNVDAMDYTYSYKAVTTNTVARASVLAAENTATGMSVSISFAANSGKVTAADSNNLAKAIRDQGFTITTSTQNGKVVYGLELNGSDAKANYTVSTVSMEAGTFKILNSNKQVVASVTISTTASFKKVSGMSQIVTAYSVSAAYATGDVYSLYDADGNAISANKLDKYFTAGGATEAGGIATTLSANSGVPKVYDVLGKEVSAVSIASTLVTAVKDKTAALKMNFHVGADGTDNNKIKINIEAMTAKSLGVNGLKVSGSSGTNATNAIEIIAGAIKKVSTQRSALGAVQNRLEHTINNLDNIVENTTAAESGIRDTDMATEMVKYSNANILSQAGQSMLAQSNQSNQGVLQLLQ

SEQ ID NO: 3 - protein, 459 aa, Unknown
  Description of Unknown Sequencefull-length Flagellin X from C3H/HeJ Bir mouse cecal bacteria genomic DNA random shear expression library PCR amplification
MVVQHNLRAMNSNRMLGITQGSLNKSTEKLSSGYKVNRAADDAAGLSISEKMRKQIRGLSQASLNAEDGISAVQTAEGALTEVHDMLQRMNELAVKAANGTNSTSDRQTIQDEVDQLLTEIDRVAETTKFNELYTLKGDEDKVTRYLSAHDAGIEGTLTQGATNATFSMDQLKFGDTIMIAGREYHISGTKAEQAAIITASVKIGQQVTIDGIMYTCSSVSNADKFELKSEDLIAKLDTSSLSIMSVNGKTYYGAGITDDRTVVSSIGAYKLIQKELGLASSIGADGATQASVNAGVDGKTLMKPSFEGKWVFSIDKGSVQVREDIDFSLHVGADADMNNKIAVKIGALDTKGLGIQGLNVKDTTGAAATYAIDSIADAVARISAQRSLLGAVQNRLEHTINNLDNVVENTTAAESQIRDTDMATEMVKYSNNNVLAQAGQSMLAQSNQANQGVLQLLQ

(19) United States
(12) Patent Application Publication
(10) Pub. No.: US 2017/0087188 A1
(43) Pub. Date: Mar. 30, 2017

(54) WNT INDUCED MOTILITY AND ENHANCED ENGRAFTMENT OF CELLS

(71) Applicant: OTTAWA HOSPITAL RESEARCH INSTITUTE, Ottawa (CA)

(72) Inventors: Michael A. RUDNICKI, Ottawa (CA); Conrad Florian BENTZINGER, Bremblens (CH)

(21) Appl. No.: 15/126,760

(22) PCT Filed: Mar. 27, 2015
(86) PCT No.: PCT/US15/22992

§ 371 Date: Sep. 16, 2016

Related U.S. Application Data
(60) Provisional application No. 61/972,097, filed on Mar. 28, 2014.

(51) Int. Cl. A61K 35/34; C12N 5/077

(52) U.S. Cl. CPC A61K 35/34; C12N 5/0658; C12N 5/0659; C12N 2501/415; A61K 48/00

(57) ABSTRACT

The invention provides cell therapy compositions and associated methods. In particular embodiments, improved therapeutic cells and improved cell-based gene therapies for promoting cell or tissue formation, regeneration, repair or maintenance in a subject in need thereof are provided.

### CROSS-REFERENCE TO RELATED APPLICATIONS

This application claims the benefit under 35 U.S.C. §119(e) of U.S. Provisional Application No. 61/972,097, filed Mar. 28, 2014, which is incorporated by reference in its entirety.

### STATEMENT OF GOVERNMENTAL SUPPORT

This work was supported in part by grants from the National Institutes of Health (R01 AR044031). The Government has certain rights in this invention.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is FATE_123_01WO_ST25.txt. The text file is 248 KB, was created on Mar. 26, 2015, and is being submitted electronically via EFS-Web.

### BACKGROUND

Technical Field

The invention relates generally to cell therapy compositions and associated methods. In particular, the present invention relates to improved therapeutic cells and improved cell-based gene therapies for promoting cell or tissue formation, regeneration, repair or maintenance in a subject in need thereof.

Description of the Related Art

Stem cells are undifferentiated or immature cells that are capable of giving rise to multiple specialized cell types and ultimately, to terminally differentiated cells. Most adult stem cells are lineage-restricted and are generally referred to by their tissue origin. Unlike any other cells, stem cells are able to renew themselves such that a virtually endless supply of mature cell types can be generated when needed over the lifetime of an organism. Due to this capacity for self-renewal, stem cells have been the subject of intense, yet disappointing, research efforts for cell or tissue regeneration, repair, and maintenance.

In several diseases and conditions affecting muscle, a reduction in muscle mass is seen that is associated with reduced numbers of satellite cells and a reduced ability of the satellite cells to repair, regenerate and grow skeletal muscle. A few exemplary diseases and conditions affecting muscle include wasting diseases, such as cachexia, muscular attenuation or atrophy, including sarcopenia, ICU-induced weakness, surgery-induced weakness (e.g., following knee or hip replacement), muscle trauma, muscle injury, surgery, disuse atrophy, and muscle degenerative diseases, such as muscular dystrophies. Muscular dystrophies are genetic diseases characterized by progressive weakness and degeneration of the skeletal or voluntary muscles which control movement. The muscles of the heart and some other involuntary muscles are also affected in some forms of muscular dystrophy. In many cases, the histological picture shows variation in fiber size, muscle cell necrosis and regeneration, and often proliferation of connective and adipose tissue. The progressive muscular dystrophies include at least Duchenne muscular dystrophy (DMD), Becker muscular dystrophy (BMD), Emery-Dreifuss muscular dystrophy, Landouzy-Dejerine muscular dystrophy, facioscapulohumeral muscular dystrophy (FSH), Limb-Girdle muscular dystrophies, von Graefe-Fuchs muscular dystrophy, oculopharyngeal muscular dystrophy (OPMD), Myotonic dystrophy (Steinert's disease) and congenital muscular dystrophies.

Satellite cells represent a heterogeneous population composed of stem cells and small mononuclear progenitor cells found in mature muscle tissue (Kuang et al., 2007). Satellite cells are involved in the normal growth of muscle, as well as the regeneration of injured or diseased tissue. In undamaged muscle, the majority of satellite cells are quiescent, meaning they neither differentiate nor undergo cell division. Satellite cells are attractive candidates for stem cell therapy of diseases and conditions affecting muscle but the therapeutic potential of satellite cells as a cell-based therapy is far from being realized.

In fact, despite significant research efforts, such therapies for skeletal muscle tissue have not yet reached the clinic (Bareja and Billin, 2013). Difficulties in obtaining sufficient donor cells, poor survival, poor engraftment and dispersal of transplanted cells in muscle tissue are fundamental problems that have not yet been resolved (Bentzinger et al., 2012).

### BRIEF SUMMARY

The present invention contemplates improved cell therapy compositions and methods. In various embodiments, cells having increased Wnt signaling are provided. In various preferred embodiments cells having increased non-canonical Wnt signaling are provided. In various other preferred embodiments, cells having increased Wnt7a signaling are provided. In particular embodiments, the cell has been contacted with or comprises one or more non-canonical Wnt signaling pathway activators to increase non-canonical Wnt signaling in the cell for a time sufficient to increase one or more therapeutic properties of the cells. The therapeutic cell may further comprise one or more exogenous polynucleotides that provide a secreted therapeutic protein and/or that provide a gene therapy.

In various embodiments, a method of increasing engraftment of a cell is provided, comprising: contacting the cell with or introducing into the cell one or more non-canonical Wnt signaling activators in vitro, for a time sufficient to increase non-canonical Wnt signaling in the cell; and administering the contacted cell to a subject in need thereof, wherein the administered cell has an increased engraftment potential compared to a non-contacted cell.

In particular embodiments, the cell is a stem cell or progenitor cell.

In certain embodiments, the stem cell is an embryonic stem cell (ESC) or an induced pluripotent stem cell (iPSC).

In further embodiments, the cell is a myogenic cell.

In some embodiments, the cell is a muscle satellite stem cell.

In additional embodiments, the myogenic cell is differentiated from an ESC or an iPSC.

In certain embodiments, the myogenic cell is a Pax7+/Myf5− cell or a Pax7+/Myf5+ cell.

In particular embodiments, the myogenic cell is a myoblast cell.

In some embodiments, the myogenic cell is a Pax7+/Myf5+/MyoD+ cell.

In additional embodiments, the cell is allogeneic to the subject.

In further embodiments, the subject and the cell are HLA compatible.

In some embodiments, the cell is not a hematopoietic cell.

In certain embodiments, the cell is genetically modified.

In additional embodiments, the non-canonical Wnt signaling activator is selected from the group consisting of a small molecule, a nucleic acid, a polypeptide, and suitable combinations thereof.

In particular embodiments, the polypeptide comprises a non-canonical Wnt polypeptide or modified non-canonical Wnt polypeptide.

In particular embodiments, the modified non-canonical Wnt polypeptide comprises one or more N-terminal or C-terminal truncations, or one or more amino acid additions, deletions, or substitutions.

In further embodiments, the modified non-canonical Wnt polypeptide comprises a biologically active fragment of the Wnt polypeptide.

In additional embodiments, the lipidation of the modified non-canonical Wnt polypeptide is reduced.

In certain embodiments, the non-canonical Wnt polypeptide comprises a Wnt7a polypeptide.

In some embodiments, the polypeptide is a Fzd7 polypeptide or modified Fzd7 polypeptide.

In particular embodiments, engraftment potential is increased by an increase in cell motility, cell migration, myofusion or a combination thereof.

In various embodiments, a myogenic cell-based gene therapy is provided, comprising a myogenic cell comprising an exogenous polynucleotide; contacting the myogenic cell in vitro with at least one non-canonical Wnt signaling activator for a time sufficient to increase non-canonical Wnt signaling in the cell; and administering the contacted myogenic cell to a subject in need of gene therapy, wherein fusion of the myogenic cell with a myofiber in the subject delivers the polynucleotide to the subject.

In certain embodiments, the cell is a muscle satellite stem cell.

In some embodiments, the myogenic cell is differentiated from an ESC or iPSC.

In further embodiments, the myogenic cell is a stem cell or a progenitor cell.

In particular embodiments, the myogenic cell is a Pax7+/Myf5− cell or a Pax7+/Myf5+ cell.

In additional embodiments, the myogenic cell is a myoblast cell.

In some embodiments, the myogenic cell is a Pax7+/Myf5+/MyoD+ cell.

In certain embodiments, the myogenic cell is allogeneic to the subject.

In additional embodiments, the subject and the myogenic cell are HLA compatible.

In some embodiments, the non-canonical Wnt signaling activator is selected from the group consisting of a small molecule, a nucleic acid, a polypeptide, and suitable combinations thereof.

In additional embodiments, the polypeptide comprises a non-canonical Wnt polypeptide or modified non-canonical Wnt polypeptide.

In particular embodiments, the modified non-canonical Wnt polypeptide comprises one or more N-terminal or C-terminal truncations, or one or more amino acid additions, deletions, or substitutions.

In certain embodiments, the modified non-canonical Wnt polypeptide comprises a biologically active fragment of the Wnt polypeptide.

In some embodiments, the lipidation of the modified non-canonical Wnt polypeptide is reduced.

In certain embodiments, the non-canonical Wnt polypeptide comprises a Wnt7a polypeptide.

In further embodiments, the polypeptide is a Fzd7 polypeptide or modified Fzd7 polypeptide.

In particular embodiments, the exogenous polynucleotide comprises a nucleic acid that encodes dystrophin, Wnt1, Wnt2, Wnt2b/13, Wnt3, Wnt3a, Wnt4, Wnt5a, Wnt5b, Wnt6, Wnt7a, Wnt7b, Wnt8, Wnt8a, Wnt8b, Wnt9a, Wnt9b, Wnt10a, Wnt10b, Wnt11, or Wnt16.

In certain embodiments, the subject has a disorder selected from the group consisting of: cachexia, cancer, AIDS, muscular attenuation, muscle atrophy, muscle trauma, muscle injury, surgery, disuse atrophy, or a muscle degenerative disease.

In further embodiments, the subject has a disorder selected from the group consisting of: Duchenne muscular dystrophy (DMD), Becker muscular dystrophy (BMD), Emery-Dreifuss muscular dystrophy, Landouzy-Dejerine muscular dystrophy, facioscapulohumeral muscular dystrophy (FSH), Limb-Girdle muscular dystrophies, von Graefe-Fuchs muscular dystrophy, oculopharyngeal muscular dystrophy (OPMD), Myotonic dystrophy (Steinert's disease) and congenital muscular dystrophies.

In various embodiments, a method for delivering a polynucleotide encoding a polypeptide-of-interest to muscle tissue of a mammal is provided, comprising contacting a myogenic cell with a non-canonical Wnt signaling activator in vitro, wherein the myogenic cell comprises a polynucleotide encoding a polypeptide-of-interest; and administering the contacted myogenic cell to a subject in need thereof, wherein the polynucleotide is delivered to the muscle tissue of the mammal.

In particular embodiments, the mammal is a human.

In additional embodiments, the myogenic cell is muscle satellite stem cell.

In certain embodiments, the myogenic cell is differentiated from an ESC or an iPSC.

In certain embodiments, wherein the myogenic cell is a stem cell or progenitor cell.

In some embodiments, the myogenic cell is a Pax7+/Myf5− cell or a Pax7+/Myf5+ cell.

In further embodiments, the myogenic cell is a myoblast cell.

In further embodiments, the myogenic cell is a Pax7+/Myf5+/MyoD+ cell.

In some embodiments, the myogenic cell is allogeneic to the subject.

In particular embodiments, the subject and the myogenic cell are HLA compatible.

In certain embodiments, the non-canonical Wnt signaling activator is selected from the group consisting of a small molecule, a nucleic acid, a polypeptide, and suitable combinations thereof.

In additional embodiments, the polypeptide comprises a non-canonical Wnt polypeptide or modified non-canonical Wnt polypeptide.

In additional embodiments, the modified non-canonical Wnt polypeptide comprises one or more N-terminal or C-terminal truncations, or one or more amino acid additions, deletions, or substitutions.

In particular embodiments, the modified non-canonical Wnt polypeptide comprises a biologically active fragment of the Wnt polypeptide.

In particular embodiments, the lipidation of the modified non-canonical Wnt polypeptide is reduced.

In additional embodiments, the non-canonical Wnt polypeptide comprises a Wnt7a polypeptide.

In further embodiments, the polypeptide is a Fzd7 polypeptide or modified Fzd7 polypeptide.

In certain embodiments, the polynucleotide comprises a nucleic acid that encodes dystrophin, Wnt1, Wnt2, Wnt2b/13, Wnt3, Wnt3a, Wnt4, Wnt5a, Wnt5b, Wnt6, Wnt7a, Wnt7b, Wnt8, Wnt8a, Wnt8b, Wnt9a, Wnt9b, Wnt10a, Wnt10b, Wnt11, or Wnt16.

In particular embodiments, the subject has a disorder selected from the group consisting of: cachexia, cancer, AIDS, muscular attenuation, muscle atrophy, muscle trauma, muscle injury, surgery, disuse atrophy, or a muscle degenerative disease.

In some embodiments, the subject has a disorder selected from the group consisting of: Duchenne muscular dystrophy (DMD), Becker muscular dystrophy (BMD), Emery-Dreifuss muscular dystrophy, Landouzy-Dejerine muscular dystrophy, facioscapulohumeral muscular dystrophy (FSH), Limb-Girdle muscular dystrophies, von Graefe-Fuchs muscular dystrophy, oculopharyngeal muscular dystrophy (OPMD), Myotonic dystrophy (Steinert's disease) and congenital muscular dystrophies.

In various embodiments, a method of increasing cell graft efficacy is provided, comprising contacting a cell graft in vitro with a non-canonical Wnt signaling activator for a time sufficient to increase the engraftment potential of the cell graft; and administering the contacted cell graft to a subject in need thereof, wherein the administered cell graft has increased engraftment compared to a non-contacted cell graft.

In additional embodiments, the cell graft comprises stem cell or progenitor cells.

In certain embodiments, the stem cells comprise ESCs or iPSCs.

In certain embodiments, the cell graft comprises myogenic cells.

In particular embodiments, the cell graft comprises muscle satellite stem cells.

In some embodiments, the myogenic cells are differentiated from ESCs or iPSCs.

In some embodiments, the myogenic cells comprise Pax7+/Myf5− cells or Pax7+/Myf5+ cells.

In further certain embodiments, the myogenic cells comprise myoblast cells.

In additional embodiments, the myogenic cells comprise Pax7+/Myf5+/MyoD+ cells.

In certain embodiments, the cell graft is allogeneic to the subject.

In some embodiments, the subject and the cell graft are HLA compatible.

In some embodiments, the cell graft comprises genetically modified cells.

In particular embodiments, the non-canonical Wnt signaling activator is selected from the group consisting of a small molecule, a nucleic acid, a polypeptide, and suitable combinations thereof.

In certain embodiments, the polypeptide comprises a non-canonical Wnt polypeptide or modified non-canonical Wnt polypeptide.

In additional embodiments, the modified non-canonical Wnt polypeptide comprises one or more N-terminal or C-terminal truncations, or one or more amino acid additions, deletions, or substitutions.

In additional embodiments, the modified non-canonical Wnt polypeptide comprises a biologically active fragment of the Wnt polypeptide.

In certain embodiments, the lipidation of the modified non-canonical Wnt polypeptide is reduced.

In further embodiments, the non-canonical Wnt polypeptide comprises a Wnt7a polypeptide.

In some embodiments, the polypeptide is a Fzd7 polypeptide or modified Fzd7 polypeptide.

In particular embodiments, the genetically modified cells comprise a polynucleotide that comprises a nucleic acid that encodes dystrophin, Wnt1, Wnt2, Wnt2b/13, Wnt3, Wnt3a, Wnt4, Wnt5a, Wnt5b, Wnt6, Wnt7a, Wnt7b, Wnt8, Wnt8a, Wnt8b, Wnt9a, Wnt9b, Wnt10a, Wnt10b, Wnt11, or Wnt16.

In some embodiments, the subject has a disorder selected from the group consisting of: cachexia, cancer, AIDS, muscular attenuation, muscle atrophy, muscle trauma, muscle injury, surgery, disuse atrophy, or a muscle degenerative disease.

In further embodiments, the subject has a disorder selected from the group consisting of: Duchenne muscular dystrophy (DMD), Becker muscular dystrophy (BMD), Emery-Dreifuss muscular dystrophy, Landouzy-Dejerine muscular dystrophy, facioscapulohumeral muscular dystrophy (FSH), Limb-Girdle muscular dystrophies, von Graefe-Fuchs muscular dystrophy, oculopharyngeal muscular dystrophy (OPMD), Myotonic dystrophy (Steinert's disease) and congenital muscular dystrophies.

In certain embodiments, engraftment is increased by an increases in cell motility, cell migration, myofusion or a combination thereof.

In various embodiments, a culture is provided, comprising a population of myogenic cells; and an exogenous non-canonical Wnt signaling pathway activator in an amount sufficient to increase the engraftment potential of the population of cells.

In particular embodiments, the population of myogenic cells comprises Pax7+/Myf5−/MyoD− cells, Pax7+/Myf5+/MyoD− cells, and/or Pax7+/Myf5+/MyoD+ cells.

In some embodiments, the population of myogenic cells is differentiated from ESCs or iPSCs.

In certain embodiments, the population of myogenic cells comprises stem cells.

In certain embodiments, the population of myogenic cells comprises muscle satellite stem cells.

In additional embodiments, the population of myogenic cells comprises progenitor cells.

In further embodiments, the population of myogenic cells comprises myoblast cells.

In some embodiments, the non-canonical Wnt signaling activator is selected from the group consisting of a small molecule, a nucleic acid, a polypeptide, and suitable combinations thereof.

In particular embodiments, the polypeptide comprises a non-canonical Wnt polypeptide or modified non-canonical Wnt polypeptide.

In further embodiments, the modified non-canonical Wnt polypeptide comprises one or more N-terminal or C-terminal truncations, or one or more amino acid additions, deletions, or substitutions.

In certain embodiments, the modified non-canonical Wnt polypeptide comprises a biologically active fragment of the Wnt polypeptide.

In further embodiments, the lipidation of the modified non-canonical Wnt polypeptide is reduced.

In additional embodiments, the non-canonical Wnt polypeptide comprises a Wnt7a polypeptide.

In some embodiments, the polypeptide is a Fzd7 polypeptide or modified Fzd7 polypeptide.

In particular embodiments, at least a portion of the population of myogenic cells is genetically modified.

In additional embodiments, the portion of the population of myogenic cells is genetically modified with a polynucleotide comprising a nucleic acid that encodes dystrophin, Wnt1, Wnt2, Wnt2b/13, Wnt3, Wnt3a, Wnt4, Wnt5a, Wnt5b, Wnt6, Wnt7a, Wnt7b, Wnt8, Wnt8a, Wnt8b, Wnt9a, Wnt9b, Wnt10a, Wnt10b, Wnt11, or Wnt16.

In various embodiments, a method of preventing, ameliorating, or treating a muscle disorder in a mammal in need thereof is provided, comprising contacting a myogenic cell comprising a polynucleotide encoding a polypeptide-of-interest with one or more non-canonical Wnt signaling activators, in vitro; and administering the contacted myogenic cell to the mammal.

In further embodiments, the mammal is a human.

In some embodiments, the myogenic cell is differentiated from an ESC or an iPSC.

In certain embodiments, the myogenic cell is muscle satellite stem cell.

In some embodiments, the myogenic cell is a stem cell or progenitor cell.

In particular embodiments, the myogenic cell is a Pax7+/Myf5− cell or a Pax7+/Myf5+ cell.

In further particular embodiments, the myogenic cell is a myoblast cell.

In additional embodiments, the myogenic cell is a Pax7+/Myf5+/MyoD+ cell.

In further embodiments, the myogenic cell is allogeneic to the subject.

In some embodiments, the subject and the myogenic cell are HLA compatible.

In additional embodiments, the non-canonical Wnt signaling activator is selected from the group consisting of a small molecule, a nucleic acid, a polypeptide, and suitable combinations thereof.

In certain embodiments, the polypeptide comprises a non-canonical Wnt polypeptide or modified non-canonical Wnt polypeptide.

In particular embodiments, the modified non-canonical Wnt polypeptide comprises one or more N-terminal or C-terminal truncations, or one or more amino acid additions, deletions, or substitutions.

In some embodiments, the modified non-canonical Wnt polypeptide comprises a biologically active fragment of the Wnt polypeptide.

In further embodiments, the lipidation of the modified non-canonical Wnt polypeptide is reduced.

In certain embodiments, the non-canonical Wnt polypeptide comprises a Wnt7a polypeptide.

In additional embodiments, the polypeptide is a Fzd7 polypeptide or modified Fzd7 polypeptide.

In further embodiments, the polynucleotide comprises a nucleic acid that encodes dystrophin, Wnt1, Wnt2, Wnt2b/13, Wnt3, Wnt3a, Wnt4, Wnt5a, Wnt5b, Wnt6, Wnt7a, Wnt7b, Wnt8, Wnt8a, Wnt8b, Wnt9a, Wnt9b, Wnt10a, Wnt10b, Wnt11, or Wnt16.

In certain embodiments, the subject has a disorder selected from the group consisting of: cachexia, cancer, AIDS, muscular attenuation, muscle atrophy, muscle trauma, muscle injury, surgery, disuse atrophy, or a muscle degenerative disease.

In particular embodiments, the subject has a disorder selected from the group consisting of: Duchenne muscular dystrophy (DMD), Becker muscular dystrophy (BMD), Emery-Dreifuss muscular dystrophy, Landouzy-Dejerine muscular dystrophy, facioscapulohumeral muscular dystrophy (FSH), Limb-Girdle muscular dystrophies, von Graefe-Fuchs muscular dystrophy, oculopharyngeal muscular dystrophy (OPMD), Myotonic dystrophy (Steinert's disease) and congenital muscular dystrophies.

### BRIEF DESCRIPTION OF THE SEVERAL VIEWS OF THE DRAWINGS

FIG. 1 shows a representative example of Wnt7a and Fzd7 inducing the polarization and migration of myogenic cells. (A) Morphological quantification of triangular polarized C2C12 cells upon Wnt7a stimulation and Fzd7 overexpression. Vehicle (Veh.) treated cells expressing YFP were set to 100%. Bars represent means±SEM. N≧4. p values are **p<0.01; *p<0.05. (B) Confocal images showing the localization of Fzd7-YFP and the Tubulin cytoskeleton of a C2C12 cell. Scale bar=4 μm. (C) Sequences derived from live-imaging of C2C12 cells that were transfected with Fzd7-YFP or YFP at the given time points. The arrowheads show peripheral Fzd7-YFP that is dynamically rearranged during cell migration. Scale bar=10 μm. (D) Frequency of peripheral Fzd7-YFP accumulation in C2C12 cells when compared to Fzd3-YFP. Fzd7-YFP was set to 100%. Bars represent means±SEM. N=3. p value is **p<0.01. (E) Representative images from scratch assays with C2C12 cells. The dashed line represents the border of the scratch wound. Cells that were treated with Wnt7a migrate further than Veh. treated cells. (F) Quantification of C2C12 migration in scratch assays as shown in (E). Wnt7a significantly increases migration compared to Veh. Bars represent means±SEM. N=3. p value is **p<0.01. (G) Overexpression of Fzd7-Flag also increases the migration of C2C12 cells in scratch assays when compared to empty vector (EV). Bars represent means±SEM. N=3. p value is **p<0.01.

FIG. 2 shows a representative example of Wnt7a and Fzd7 facilitating directed cell migration. (A) Scratch migration assay with mouse primary myoblasts that were stimulated with Wnt7a or Veh. Bars represent means±SEM. N=3. p value is *p<0.05. (B) Quantification of scratch assays using primary myoblasts overexpressing EV or Fzd7-Flag. Bars represent means±SEM. N=3. p value is ***p<0.001. (C) Primary myoblasts derived from Fzd7 knockout mice (Fzd7−/−) do not respond to Wnt7a stimulation. Bars represent means±SEM. N≧3. No significant difference (n.s.). (D) Scratch migration assay showing that Fzd−/− primary myoblasts migrate significantly less than heterozygous cells (Fzd7+/−). Genetic Fzd7 knockout can be rescued by expression of Fzd7-Flag. Bars represent means±SEM. N=3. p value is ***p<0.001. (E) Quantification showing that canonical Wnt3a does not affect cell migration in scratch wound assays. Bars represent means±SEM. N=3. (F) Mean velocity of satellite cells on single cultured myofibers as determined by live-imaging. Bars represent means±SEM. N≧27. p value is *p<0.05. (G) The mean maximal speed of Wnt7a stimulated satellite cells is not significantly different from the Veh. control. Bars represent means±SEM. N≧27. (H) Quantification of the mean change in direction of Wnt7a treated satellite cells. In the presence of Wnt7a the cells migrate with increased directional persistence when compared to the Veh. Bars represent means±SEM. N≧27. p value is ***p<0.001. (I) Representative tracks of satellite cells on single cultured myofibers. The green x represents the start of imaging while the blue x is the stop. Fewer changes in directional motility can be observed for the Wnt7a treated satellite cell.

FIG. 3 shows the involvement of Dvl2 and Rac1 in Wnt7a induced cell migration. (A) Fzd7-tdTomato colocalizes with GFP-Rac1 (Arrowhead) in the periphery of C2C12 cells but not in cytoplasmatic vesicles. Scale bar=5 μm. (B) Rac1 activation assay of mouse primary myoblasts transduced with Wnt7a-HA (Wnt7a) retrovirus or an empty control virus (EV). In addition, all cells were either treated with an siRNA SMARTpool targeting Dvl2 (siDvl2) or with a scrambled control (siSCR). Total Rac1 is shown as a loading control. (C) Co-IP of Rac1 with Dvl2 in primary myoblasts that were infected with Wnt7a or EV. More Rac1 associates with Dvl2 in Wnt7a expressing cells. (D) Scratch assay with mouse primary myoblasts that were Wnt7a or Veh. treated. The cells were also transfected with either siSCR or siDvl2. Bars represent means±SEM. N=3. p value is *p<0.05. (E) Scratch assay with mouse primary myoblasts that overexpress EV or Fzd7-Flag and that were treated with siDvl2 or siSCR. Bars represent means±SEM. N=3. p value is *p<0.05. (F) Dominant negative Rac1 (Rac1-DN) prevents Wnt7a induced mouse primary myoblast migration in scratch assays. Bars represent means±SEM. N=3. p value is **p<0.01. (G) Rac1-DN prevents Fzd7-Flag induced mouse primary myoblast migration in scratch assays. Bars represent means±SEM. N=3. p value is *p<0.05.

FIG. 4 shows that the Fzd7/Wnt7a signal is non-canonical. (A) Wnt3a induces a dose dependent increase in TOP-flash luciferase reporter activity in C2C12 cells. Bars represent means±SEM. N=3. p value is *p<0.05. (B) Wnt7a does not activate the TOP-flash reporter at any tested concentration. Bars represent means±SEM. N=3. (C) Wnt7a treatment decreases the abundance of peripheral Fzd7-YFP and leads to its accumulation in intracellular clusters (arrowheads). Scale bar=5 μm. (D) Inhibition of clathrin-dependent endocytosis with monodansylcadaverine (MDC) prevents Wnt7a induced migration of primary myoblasts in scratch assays. Bars represent means±SEM. N=3. p value is ***p<0.001. (E) Substantial amounts of Wnt7a-HA are present in intracellular stores (arrowheads) 72 h after a 3 h exposure to conditioned supernatants produced in COS-1 cells (upper panel). In primary myoblasts from Fzd7−/− mice Wnt7a does not show such intracellular accumulation (lower panel). Scale bar=2 μm.

FIG. 5 shows a representative example of Wnt7a loading increasing myoblast dispersal in muscle tissue. (A) Experimental scheme for the in vivo myoblast dispersal assay. Cells expressing tdTomato were treated with Wnt7a or Veh. for 3 h, washed and transplanted into C57BL/6 mice. Blue fluorescent microspheres were co-injected to mark the injection site. After 7d, the distance from the closest microsphere to myofibers expressing high-(tdT+++), medium-(tdT++). or low-(tdT+) level of tdTomato was enumerated in muscle cross-sections. (B) The total number tdTomato-expressing myofibers generated by fusion with donor myoblasts was increased by Wnt7a treatment compared to Veh. Bars represent means±SEM. N=3. p value is *p<0.05. (C) Representative images showing the abundance and fluorescent intensity myofibers expressing tdTomato with respect to the injection site (outlined by a dashed line). The injection site is marked by a high concentration of blue fluorescent microspheres. In case of the cells that were treated with Wnt7a the myofibers are more spread out and generally express reduced levels of tdTomato. Scale bar=50 μm. (D-F) The minimal distance of tdT+++, tdT++ and tdT+ myofibers to the microspheres was measured in muscle cross-section according to the false color image shown in the respective inserts. The data for the minimal distance from microspheres of the myofiber types was grouped into 200 μm bins. In the Veh. treated condition, a large fraction of myofibers proximal to the injection site is tdT+++, while few distal tdT+ myofibers are present. The Wnt7a condition shows the opposite trend. Bars represent means±SEM. N≧3. p values are *<0.05 and **p<0.01.

FIG. 6 shows that Wnt7a-loaded satellite cells have an enhanced engraftment potential. (A) Schematic of the isolation, ex vivo or in vitro treatment and transplantation of satellite cells from Pax7-zsGreen mice. (B) Representative pictures showing engraftment of zsGreen and Pax7-expressing donor derived cells that were either Wnt7a or Veh. treated. Scale bar=20 μm. (C) Quantification of engraftment of Wnt7a or Veh. treated donor satellite cells (Pax7+/zsGreen+, Arrowheads). Bars represent means±SEM. N=3. p value is **p<0.01. (D) Representative images showing dystrophin-expressing myofibers (asterisks) derived from transplanted Wnt7a or Veh. treated satellite cells. Scale bar=20 μm. (E) Quantification of myofibers expressing dystrophin after transplantation of Wnt7a or Veh. treated satellite cells. Bars represent means±SEM. N=3. p value is **p<0.01. (F) Engrafted satellite cells form clusters of dystrophin-expressing myofibers in host mdx muscles. The photographs show the TA muscle that was injected with Wnt7a or Veh. treated cells. The distance between the two maximally spaced clusters of dystrophin-expressing myofibers (highlighted in red) is indicated with a magenta line. (G) Quantification of the maximal cluster distance in muscles injected with Wnt7a or Veh. treated cells. Bars represent means±SEM. N≧4. p value is **p<0.01. (H) Minimal feret measurements of dystrophin-expressing myofibers in host muscles. Myofibers that had fused with Wnt7a treated cell become hypertrophic. Bars represent means±SEM. N=3. p value is *p<0.05. (I) Quantification of the twitch force tension of mdx muscles injected with Wnt7a or Veh. treated cells. Bars represent means±SEM. N≧7. p value is **p<0.01. (J) Maximal specific force generated by mdx muscles injected with Wnt7a or Veh. treated cells. Bars represent means±SEM. N<=7. p value is *p<0.05.

FIG. 7 shows a representative example of Wnt7a stimulating dispersal of human primary myoblasts. (A) Scratch assay with human myoblasts. Wnt7a treatment significantly increases cell migration into the scar and expression of Rac1-DN prevents this effect. Bars represent means±SEM. N=3. p value is **p<0.01. (B) Fzd7-Flag increases human myoblast migration and Rac1-DN prevents this effect. Bars represent means±SEM. N=3. p value is **p<0.01. (C) Strategy used for Wnt7a or Veh. treatment and subsequent transplantation of human primary myoblasts into mdx mice. (D) Number of dystrophin-expressing myofibers following transplantation of Wnt7a or Veh. treated cells. Bars represent means±SEM. N≧4. p value is ***p<0.001. (E) Minimal myofiber feret of dystrophin-expressing myofibers generated from fusion with Wnt7a or Veh. treated human primary myoblasts. Bars represent means±SEM. N=3. p value is *p<0.05. (F) Mean maximum cluster difference in muscles transplanted with Wnt7a or Veh. treated human primary myoblasts. Bars represent means±SEM. N≧4. p value is **p<0.01.

FIG. 8 shows a model for the molecular mechanisms of ex vivo Wnt7a modulation. Upon stimulation, Wnt7a induces the symmetric proliferation of Myf5 independent satellite cells in conjunction with Fibronectin (FN1), Syndecan-4 (SDC4) and Vangl2 through the planar cell polarity pathway. In myogenic progenitors Wnt7a also facilitates Rac1 mediated cell polarization and migration. Fusion of Wnt7a treated cells activates the AKT-mTOR pathway leading to myofiber hypertrophy. Therefore, Wnt7a acts on three levels to facilitate the outcomes of cell therapy: (1) it boosts stem cell number, (2) facilitates their dispersion in the host tissue and (3) leads to muscle growth.

FIG. 9 shows a representative example of Wnt7a and Fzd7 polarizing myogenic cells. (A) Morphological quantification of C2C12 cells transfected with the indicated constructs and treated with either Wnt7a or Veh. Representative pictures of cell morphologies are depicted with YFP transfected cells above. Bars represent means±SEM. N≧4. p values are **p<0.01; *p<0.05. (B) Subcellular localization of YFP with respect to the tubulin cytoskeleton. Scale bar=2 μm. (C) Localization of Fzd7-YFP in primary myoblasts. Note the accumulation of Fzd7 in the cellular periphery. Scale bar=10 μm. (D) Localization of Fzd7-YFP in satellite cells that were transfected on single muscle fibers. Accumulation of Fzd7 in the cellular periphery can be observed. Scale bar=10 μm. (E) A human primary myoblast that was transfected with Fzd7-YFP. Similar to other myogenic cell types, Fzd7 shows accumulation in the periphery of the cell. Scale bar=10 μm. (F) Little to no peripheral localization could be observed for Fzd3-YFP in C2C12 cells. Scale bar=10 μm.

FIG. 10 shows representative Fzd7 mRNA levels in Fzd7 knockout muscle tissue and dose dependency of Wnt7a mediated cell migration. (A) qPCR comparing Fzd7 expression in muscles of Fzd7+/− and Fzd7−/− mice. Bars represent means±SEM. N=3. p value is *p<0.05. (B) Scratch assay using mouse primary myoblasts that were exposed to different concentrations of Wnt3a or Wnt7a. Bars represent means±SEM. N=3. p values are *p<0.05 and **p<0.01.

FIG. 11 shows a representative Grey value quantification of western blots, Dvl2 knockdown and Wnt7a endocytosis in C2C12 cells. (A) Grey value quantification of western blots for active Rac1 as shown in FIG. 3B. Bars represent means±SEM. N=3. p value is **p<0.01. (B) qPCR comparing Dvl2 expression upon siDvl2 or siSCR treatment in primary myoblasts. Bars represent means±SEM. N=3. p value is ***p<0.001. (C) Grey value quantification from western blots of Rac1 bound to Dvl2 as shown in FIG. 3C. Bars represent means±SEM. N=5. p value is *p<0.05. (D) Scratch assay using Wnt7a stimulated mouse primary myoblasts expressing either EV, RhoA-DN or Cdc42-DN. Bars represent means±SEM. N=3. p value is **p<0.01. (E) A C2C12 cell that was loaded for three hours with Wnt7a-HA from conditioned medium produced in COS-1 cells, washed extensively and then cultured for >72 hours. Wnt7a was detected by staining for the HA epitope. Scale bar=10 μm.

FIG. 12 shows a representative example of the effects of Wnt7a loading on cell cycle, engraftment and number of endogenous satellite cells. (A&B) The proliferation of equal numbers of primary myoblasts in the presence of different concentrations of Wnt7a and Wnt3a was assayed over five days. Data points represent means±SEM. N=3. p value is *p<0.05. (C) Immunostaining for engrafted zsGreen positive satellite cells (full arrowheads) upon Wnt7a and Veh. treatment. Host derived satellite cells are negative for zsGreen (empty arrowhead). Scale bar=50 μm. (D) Pax7+/zsGreen+ engrafted satellite cells that were treated with Wnt7a do not show more Ki67 staining than Veh. treated cells. Bars represent means±SEM. N=3. (E) The number of endogenous Pax7+/zsGreen− satellite cells is not significantly changed by transplantation of Wnt7a or Veh. treated satellite cells. Bars represent means±SEM. N=3.

FIG. 13 shows a representative example of Wnt7a loading improving the engraftment of mouse primary myoblasts. (A) Strategy used for Wnt7a or Veh. treatment and subsequent transplantation of mouse primary myoblasts into mdx mice. (B) Number of dystrophin positive fibers upon transplantation of Wnt7a or Veh. treated mouse primary myoblasts. Bars represent means±SEM. N=3. p value is *p<0.05. (C) Minimal fiber feret of dystrophin positive fibers generated from fusion with Wnt7a or Veh. treated mouse primary myoblasts. Bars represent means±SEM. N=3. p value is **p<0.01. (D) Mean maximum cluster distance in muscles transplanted with Wnt7a or Veh. treated mouse primary myoblasts. Bars represent means±SEM. N=3. p value is *p<0.05. (E&F) Three weeks following transplantation of Wnt3a and Wnt5a treated zsGreen+ myoblasts, no difference in the number of dystrophin positive fibers or in the mean maximal cluster distance is observed when compared to Veh. Bars represent means±SEM. N=3.

### BRIEF DESCRIPTION OF THE SEQUENCE IDENTIFIERS

SEQ ID NO: 1 sets forth a cDNA sequence of human Wnt7a.

SEQ ID NO: 2 sets forth the amino acid sequence of the human Wnt7a polypeptide encoded by SEQ ID NO: 1.

SEQ ID NO: 3 sets forth the amino acid sequence of the human Wnt7a polypeptide of SEQ ID NO: 2, having an alanine mutation at amino acid position 206.

SEQ ID NO: 4 sets forth the amino acid sequence of a mouse Wnt7a polypeptide.

SEQ ID NO: 5 sets forth the amino acid sequence of a rat Wnt7a polypeptide.

SEQ ID NO: 6 sets forth the amino acid sequence of a chicken Wnt7a polypeptide.

SEQ ID NO: 7 sets forth the amino acid sequence of a zebrafish Wnt7a polypeptide.

SEQ ID NO: 8 sets forth the amino acid sequence of a porcine Wnt7a polypeptide.

SEQ ID NO: 9 sets forth the amino acid sequence of a bovine Wnt7a polypeptide.

SEQ ID NO: 10 sets forth the amino acid sequence of a human Wnt7a polypeptide with the native secretion signal peptide replaced with the signal peptide of Human Immunoglobulin Kappa Chain.

SEQ ID NO: 11 sets forth a cDNA sequence of human Wnt5a.

SEQ ID NO: 12 sets forth the amino acid sequence of the human Wnt5a polypeptide encoded by SEQ ID NO: 14.

SEQ ID NO: 13 sets forth the amino acid sequence of the human Wnt5a polypeptide of SEQ ID NO: 15, having an alanine mutation at amino acid position 104.

SEQ ID NO: 14 sets forth the amino acid sequence of the human Wnt5a polypeptide of SEQ ID NO: 15, having an alanine mutation at amino acid position 244.

SEQ ID NO: 15 sets forth the amino acid sequence of the human Wnt5a polypeptide of SEQ ID NO: 15, having an alanine mutation at amino acid position 104 and at position 244.

SEQ ID NO: 16 sets forth the amino acid sequence of a mouse Wnt5a polypeptide.

SEQ ID NO: 17 sets forth the amino acid sequence of a rat Wnt5a polypeptide.

SEQ ID NO: 18 sets forth the amino acid sequence of a chicken Wnt5a polypeptide.

SEQ ID NO: 19 sets forth the amino acid sequence of a zebrafish Wnt5a polypeptide.

SEQ ID NO: 20 sets forth the amino acid sequence of a bovine Wnt5a polypeptide.

SEQ ID NO: 21 sets forth the amino acid sequence of a human Wnt1 polypeptide.

SEQ ID NO: 22 sets forth the amino acid sequence of a human Wnt2 polypeptide.

SEQ ID NO: 23 sets forth the amino acid sequence of a human Wnt2b polypeptide.

SEQ ID NO: 24 sets forth the amino acid sequence of a human Wnt3 polypeptide.

SEQ ID NO: 25 sets forth the amino acid sequence of a human Wnt3a polypeptide.

SEQ ID NO: 26 sets forth the amino acid sequence of a human Wnt4 polypeptide.

SEQ ID NO: 27 sets forth the amino acid sequence of a human Wnt5b polypeptide.

SEQ ID NO: 28 sets forth the amino acid sequence of a human Wnt6 polypeptide.

SEQ ID NO: 29 forth the amino acid sequence of a human Wnt7b polypeptide.

SEQ ID NO: 30 sets forth the amino acid sequence of a human Wnt8a polypeptide.

SEQ ID NO: 31 sets forth the amino acid sequence of a human Wnt8b polypeptide.

SEQ ID NO: 32 sets forth the amino acid sequence of a human Wnt9a polypeptide.

SEQ ID NO: 33 sets forth the amino acid sequence of a human Wnt9b polypeptide.

SEQ ID NO: 34 sets forth the amino acid sequence of a human Wnt10a polypeptide.

SEQ ID NO: 35 sets forth the amino acid sequence of a human Wnt10b polypeptide.

SEQ ID NO: 36 sets forth the amino acid sequence of a human Wnt11 polypeptide.

SEQ ID NO: 37 sets forth the amino acid sequence of a human Wnt16 polypeptide.

SEQ ID NO: 38 sets forth amino acids 32-212 of SEQ ID NO: 2.

SEQ ID NO: 39 sets forth amino acids 213-349 of SEQ ID NO: 2.

SEQ ID NO: 40 sets forth amino acids 221-349 of SEQ ID NO: 2.

SEQ ID NO: 41 sets forth amino acids 235-349 of SEQ ID NO: 2.

SEQ ID NO: 42 sets forth amino acids 264-349 of SEQ ID NO: 2.

SEQ ID NOs: 43-46 set forth the amino acid sequences of fusion polypeptides comprising the amino acid sequence of SEQ ID NO: 40.

SEQ ID NOs: 47-50 set forth the amino acid sequences of fusion polypeptides comprising the amino acid sequence of SEQ ID NO: 41.

SEQ ID NOs: 51-54 set forth the amino acid sequences of fusion polypeptides comprising the amino acid sequence of SEQ ID NO: 42.

SEQ ID NOs: 55-57 set forth polynucleotide sequences used to construct Wnt expression vectors.

SEQ ID NO: 58 sets forth the polynucleotide sequence that encodes a CD33 signal peptide.

SEQ ID NO: 59 sets forth the amino acid sequence encoded by the polynucleotide sequence of SEQ ID NO: 60.

SEQ ID NO: 60 sets forth the polynucleotide sequence that encodes a IgGκ signal peptide.

SEQ ID NO: 61 sets forth the amino acid sequence encoded by the polynucleotide sequence of SEQ ID NO: 62

SEQ ID NOs: 62-63 set forth the amino acid sequences of fusion polypeptides comprising the amino acid sequence of SEQ ID NO: 40.

SEQ ID NOs: 64-65 set forth the amino acid sequences of fusion polypeptides comprising the amino acid sequence of SEQ ID NO: 41.

SEQ ID NOs: 66-67 set forth the amino acid sequences of fusion polypeptides comprising the amino acid sequence of SEQ ID NO: 42.

SEQ ID NOs: 68-69 set forth the amino acid sequences of fusion polypeptides comprising the amino acid sequence of SEQ ID NO: 41.

SEQ ID NOs: 70-71 set forth the amino acid sequences of fusion polypeptides comprising the amino acid sequence of SEQ ID NO: 42.

SEQ ID NOs: 72-73 set forth the amino acid sequences of fusion polypeptides comprising the amino acid sequence of SEQ ID NO: 41.

SEQ ID NOs: 74-75 set forth the amino acid sequences of fusion polypeptides comprising the amino acid sequence of SEQ ID NO: 42.

SEQ ID NO: 76 sets forth the amino acid sequence of human FZD1.

SEQ ID NO: 77 sets forth the amino acid sequence of human FZD2.

SEQ ID NO: 78 sets forth the amino acid sequence of human FZD3.

SEQ ID NO: 79 sets forth the amino acid sequence of human FZD4.

SEQ ID NO: 80 sets forth the amino acid sequence of human FZD5.

SEQ ID NO: 81 sets forth the amino acid sequence of human FZD6.

SEQ ID NO: 82 sets forth the amino acid sequence of human FZD7.

SEQ ID NO: 83 sets forth the amino acid sequence of human FZD8.

SEQ ID NO: 84 sets forth the amino acid sequence of human FZD9.

SEQ ID NO: 85 sets forth the amino acid sequence of human FZD10.

SEQ ID NOs: 86-89 represent PCR primers.

SEQ ID NOs: 90-99 set forth cDNA sequences that encodes Fzd polypeptides.

SEQ ID NOs: 100-109 set forth amino acid sequences of various cell permeable peptides.

SEQ ID NOs: 110-111 set forth amino acid sequences of peptide linkers.

### DETAILED DESCRIPTION

### A. Overview

The present invention contemplates improved cell therapies and methods of using the same. Without wishing to be bound to any particular theory, the present inventors have discovered that increasing non-canonical Wnt signaling in a cell increases one or more therapeutic properties of the cell, and thus, results in an improved therapeutic compared to existing cell-based therapeutics. It is contemplated that cells having an increase in one or more therapeutic properties, e.g., increased motility, migration, dispersion, and/or engraftment potential, enhance the outcome of cell therapies because such cell therapies allow for delivery of small numbers of cells that provide a substantial and long-lasting therapeutic benefit including, without limitation, regenerative therapy and gene therapy.

In various embodiments, compositions comprising one or more therapeutic cells having increased Wnt signaling are provided. In various preferred embodiments, compositions comprising one or more therapeutic cells having increased non-canonical Wnt signaling are provided. In various other preferred embodiments, compositions comprising one or more therapeutic cells having increased Wnt7a signaling are provided. The therapeutic cell has been contacted with or comprises one or more non-canonical Wnt signaling pathway activators to increase non-canonical Wnt signaling in the cell for a time sufficient to increase one or more therapeutic properties of the cells. The therapeutic cell may further comprise one or more exogenous polynucleotides that provide a secreted therapeutic protein and/or that provide a gene therapy. Also contemplated are cultures comprising compositions of one or more therapeutic cells.

In various embodiments, methods of providing cell-based therapy are contemplated. In one embodiment, a method of increasing engraftment of a cell having increased non-canonical Wnt signaling is provided. Increased non-canonical Wnt signaling may be provided by contacting the cell with one or more non-canonical Wnt signaling pathway activators for a time sufficient to increase the non-canonical Wnt signaling in the cell, thereby increasing the engraftment potential of the cell. Methods of increasing the efficacy of a cell graft are also contemplated. Cell grafts may be prepared ex vivo or in vitro by contacting the graft, e.g., a population of cells, with a sufficient amount of a non-canonical Wnt signaling pathway activator for a sufficient duration to increase non-canonical Wnt signaling in the cell graft, thereby improving the engraftment potential of the cell graft.

In various embodiments, the cell-based therapy comprises gene therapy. In one embodiment, a cell comprising one or more polynucleotides encoding a therapeutic protein may be contacted with at least one non-canonical Wnt signaling pathway activator and the therapeutic cell may then be administered to a subject in need of the therapeutic protein. It is further contemplated that such gene therapies may be provided to subjects in need or regenerative therapy, a subject having any monogenetic disorder, degenerative disease, or a subject having any other disease, disorder, or condition that would be amenable to gene therapy.

In various embodiments, methods of treating, preventing, or ameliorating at least one symptom of a subject in need is provided. In one embodiment, a subject in need of treatment has, or has been diagnosed with a disorder that would benefit from increased cell or tissue regeneration or from the delivery of a therapeutic protein to an affect cell or tissue. In other embodiments, the subject in need of treatment has a degenerative disorder including, without limitation, degenerative nervous system disorders and degenerative muscular disorders, are contemplated. In one embodiment, a cell comprising an exogenous polynucleotide encoding a therapeutic protein that is deficient, reduced, or absent in a subject, is contacted with one or more non-canonical Wnt signaling pathway activators to increase the non-canonical Wnt signaling in the cell. The cell is then administered to a subject in need of the therapeutic protein.

In one embodiment, a therapeutic myogenic cell comprises increased non-canonical Wnt signaling as a result of being contacted with or cultured with one or more non-canonical Wnt signaling activators for a sufficient time to increase non-canonical Wnt signaling in the cell. In another embodiment, the therapeutic myogenic cell comprises an exogenous non-canonical Wnt signaling activator or polynucleotide encoding the same, and has optionally been genetically modified to express the non-canonical Wnt signaling activator. In yet another embodiment, the therapeutic myogenic cell has been genetically modified with a polynucleotide encoding a therapeutic protein and has optionally been genetically modified to express the non-canonical Wnt signaling activator. Without wishing to be bound to any particular theory, it is contemplated that therapeutic myogenic cells can be exploited to potentiate the outcome of a muscle cell therapy because ex vivo or in vitro modulation of the myogenic cells to increase non-canonical Wnt signaling results in increased dispersal and engraftment of transplanted cells that eventually fuse and form multinucleate syncytia with endogenous muscle cells, thereby delivering not only a wild type copy of a genome to an affected cell population but also any therapeutic proteins the myogenic cell was genetically modified to express. Thus, the present inventors have discovered an improved method of providing treatment and gene therapy to a subject in need thereof.

In particular embodiments, a culture comprising a population of myogenic cells including, but not limited to Pax7⁺/Myf5⁻/MyoD⁻, Pax7⁺/Myf5⁺/MyoD⁻, or Pax7⁺/Myf5⁺/MyoD⁺ or any combination thereof; and a non-canonical Wnt signaling pathway activator in an amount sufficient to increase the engraftment potential of the population of cells is provided.

A method of increasing engraftment of a cell comprising contacting the cell in vitro with a non-canonical Wnt signaling pathway activator for a time sufficient to increase non-canonical Wnt signaling in the cell; and administering the contacted cell to a subject in need thereof; wherein the administered cell has an increased engraftment potential compared to a non-contacted cell. In another embodiment, the cell comprises an exogenous non-canonical Wnt signaling activator or polynucleotide encoding the same, and has optionally been genetically modified to express the non-canonical Wnt signaling activator. In yet another embodiment, the cell has also been genetically modified with a polynucleotide encoding a therapeutic protein.

In certain embodiments, a method of increasing cell graft efficacy is provided. The method may comprise treating a population of cells to be transplanted, i.e., a cell graft, with one or more non-canonical Wnt signaling pathway activators for a time sufficient to increase one or more therapeutic properties of the cell graft, e.g., engraftment potential. The improved cell graft may then be administered to a subject in need thereof. In another embodiment, the cell graft has been genetically modified to express a non-canonical Wnt signaling activator and has optionally been genetically modified to express a therapeutic protein.

In various embodiments, a myogenic cell-based gene therapy is provided by contacting a myogenic cell, that has optionally been genetically modified with a polynucleotide encoding a therapeutic protein, with a non-canonical Wnt signaling activator for a time sufficient to increase non-canonical Wnt signaling in the cell. The genetically modified myogenic cell with increased non-canonical Wnt signaling is then subsequently administered to a subject in need of gene therapy. Without wishing to be bound to any particular theory, it is contemplated that the myogenic cell with increased non-canonical Wnt signaling has improved dispersal and engraftment properties and thereby delivers the polynucleotide encoding the therapeutic polypeptide to the subject when the myogenic cell fuses with a myofiber in the subject.

Also contemplated herein are methods for delivering a polynucleotide encoding a therapeutic polypeptide or polypeptide-of-interest to a subject. In one embodiment, a myogenic cell is genetically modified with a polynucleotide encoding a therapeutic polypeptide or polypeptide-of-interest and contacted, or genetically modified, with one or more non-canonical Wnt signaling activators that increase non-canonical Wnt signaling in the myogenic cell. The myogenic cell is then administering to a subject and delivers the polynucleotide encoding a therapeutic polypeptide or polypeptide-of-interest to the muscle tissue of the subject following engraftment and cell fusion of the myogenic cell with the muscle tissue of the subject.

In various embodiments, strategies to treat progressive degenerative muscle diseases is contemplated. In one embodiment a myogenic cell is genetically altered with a polynucleotide encoding a therapeutic protein and contacted with, cultured with, or modified to express a non-canonical Wnt signaling pathway activator to increase non-canonical Wnt signaling in the cell. Administration of the genetically altered myogenic cells facilitate the genetic correction of affected muscle fibers and the restoration of tissue regenerative capacity. The ability of the myogenic cells to efficiently disperse and engraft and to add their nuclei to the syncytial muscle fibers through fusion makes them an ideal cell therapy for genetic diseases that affect myofiber stability or function. In addition, such therapeutic strategies may provide a life-long muscle hypertrophy as a consequence of transplanting a small number of myogenic cells comprising Pax7⁺/Myf5⁻/MyoD⁻ cells.

In various embodiments, it is contemplated that increasing non-canonical Wnt7a/Fzd7 signaling in a myogenic cell increases one or more therapeutic properties of the cell, e.g., increased engraftment potential of the cells, and/or an increased ability of the transplanted cells to disperse from the administration site, to undergo myofusion, to increase force generation, to increase twitch tension; and/or to increase cell motility or cell migration of the cells. In one embodiment, non-canonical Wnt7a/Fzd7 signaling is increased in the cell by contacting or culturing the myogenic cell with a small molecule that increases Wnt7a/Fzd7 signaling, by contacting or culturing the myogenic cell with a Wnt7a or Fzd7 polypeptide contemplated herein, or modifying the myogenic cell to express a Wnt7a or Fzd7 polypeptide as contemplated herein. Without wishing to be bound to any particular theory, it is contemplated that increased Wnt7a/Fzd7 signaling in myogenic cells increases the polarity and directional migration of the myogenic cells through activation of Dvl2 and the small GTPase Rac1 and can be exploited to potentiate the outcome of myogenic cell transplantation and enhance the efficacy of stem cell therapy for skeletal muscle.

In particular embodiments, the practice of the invention will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Such techniques are explained fully in the literature. See, e.g., Sambrook, et al., Molecular Cloning: A Laboratory Manual (3rd Edition, 2001); Sambrook, et al., Molecular Cloning: A Laboratory Manual (2nd Edition, 1989); Maniatis et al., Molecular Cloning: A Laboratory Manual (1982); Ausubel et al., Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford, 1985); Anand, Techniques for the Analysis of Complex Genomes, (Academic Press, New York, 1992); Transcription and Translation (B. Hames & S. Higgins, Eds., 1984); Perbal, A Practical Guide to Molecular Cloning (1984); and Harlow and Lane, Antibodies, (Cold Spring Harbor Laboratory Press, Cold Spring Harbor, N.Y., 1998).

All publications, patents and patent applications cited herein are hereby incorporated by reference in their entirety.

### B. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred embodiments of compositions, methods and materials are described herein. For the purposes of the present invention, the following terms are defined below.

### A, an, the

The articles “a,” “an,” and “the” are used herein to refer to one or to more than one (i.e., to at least one) of the grammatical object of the article. By way of example, “an element” means one element or more than one element.

### About

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%, or any intervening range thereof.

### Substantially

The term “substantially” refers to a quantity, level, concentration, value, number, frequency, percentage, dimension, size, amount, weight or length that is 95%, 96%, 97%, 98%, 99% or 100% of a reference value. For example, a composition that is substantially homogeneous, e.g., a cell population, is 95%, 96%, 97%, 98%, 99% or 100% free different cells, or the different cells are undetectable as measured by conventional means. Similar meaning can be applied to the term “absence of,” where referring to the absence of a particular substance or component of a composition.

### Appreciable

As used herein, the term “appreciable” refers to a range of quantity, level, value, number, frequency, percentage, dimension, size, amount, weight or length or an event that is readily detectable by one or more standard methods. The terms “not-appreciable” and “not appreciable” and equivalents refer to a range of quantity, level, value, number, frequency, percentage, dimension, size, amount, weight or length or an event that is not readily detectable or undetectable by standard methods. In one embodiment, an event is not appreciable if it occurs less than 5%, 4%, 3%, 2%, 1%, 0.1%, 0.01%, 0.001% or less of the time.

Comprising, Consisting of Consisting essentially of

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements

### Embodiment

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

Stem cell

The term “stem cell” refers to a cell which is an undifferentiated cell capable of (1) long term self-renewal, or the ability to generate at least one identical copy of the original cell, (2) differentiation at the single cell level into multiple, and in some instance only one, specialized cell type and (3) of in vivo functional regeneration of tissues. Stem cells are subclassified according to their developmental potential as totipotent, pluripotent, multipotent and oligo/unipotent.

Pluripotent cell

A “pluripotent cell” refers a cell that has the ability to form all lineages, i.e., ectoderm, mesoderm, endoderm, of the body or soma (i.e., the embryo proper). Pluripotent cells can be identified, in part, by assessing pluripotency characteristics including, but are not limited to: (i) pluripotent stem cell morphology; (ii) the potential for unlimited self renewal (iii) expression of pluripotent stem cell markers; (iv) teratoma formation; and (v) formation of embryoid bodies. Illustrative examples of pluripotent cells include embryonic stem cells or induced pluripotent stem cells.

Adult stem cell

An “adult stem cell” or “somatic stem cell” refers to a stem cell, typically a multipotent stem cell, that is found in a developed or developing organism; often in a specific tissue of an organism. Adult stem cells can divide by cell division, are either multipotent or unipotent and subsequently differentiate to increase, replace or regenerate lost cells and/or tissues. Adult stem cells include, but are not limited to, ectodermal stem cells, endodermal stem cells, mesodermal stem cells, neural stem cells, hematopoietic stem cells, muscle stem cells, satellite stem cells, and the like.

Progenitor cells

The term “progenitor cell” refers to a cell that has the capacity to self-renew and to differentiate into more mature cells, but is committed to a lineage (e.g., hematopoietic progenitors are committed to the blood lineage), whereas stem cells are not necessarily so limited. A myoblast is an example of a progenitor cell, which is capable of differentiation to only one type of cell, but is itself not fully mature or fully differentiated. A myoblast may differentiate into a myocyte.

Satellite cells

“Satellite cells” refer to satellite stem cells, satellite progenitor cells, or populations of cells comprising a mixture of satellite stem cells and satellite progenitor cells in any particular ratio.

Satellite stem cells/progenitor cells

The term “satellite stem cell” refers to a type of adult stem cell that gives rise to cells of the myogenic lineage, e.g., satellite progenitor cells, myoblasts, and myocytes. In one embodiment, a satellite stem cell refers to a Pax7⁺ myogenic cell. In one embodiment, the satellite stem cell is a Pax7⁺/Myf5⁻ (MyoD⁻) muscle stem cell or a muscle satellite stem cell. In a particular embodiment, the satellite stem cell is a skeletal muscle stem cell. The term “satellite progenitor cell” refers to a type of progenitor cell that gives rise to myoblasts and myocytes. In one embodiment, the satellite progenitor cell is a Pax7⁺/Myf5⁺ (MyoD⁻) muscle stem cell. In a particular embodiment, the satellite stem cell is a skeletal muscle progenitor cell.

### Myoblast

The term “myoblast” refers to a muscle progenitor cell that gives rise to myocytes. In one embodiment, the myoblast is a Pax7⁺/Myf5⁺/MyoD⁺ muscle progenitor cell.

### Myocyte

The term “myocyte” or “myofiber” refers to a differentiated type of cell found in muscles. Each myocyte contains myofibrils, which are long chains of sarcomeres, the contractile units of the muscle cell. Myocytes fuse to form multinucleate syncytia in skeletal muscle. There are various specialized forms of myocytes: cardiac, skeletal, and smooth muscle cells, with various properties known in the art.

Myogenic cells

A “myogenic cell” refers to a cell of the muscle lineage or a cell that gives rise to a cell of the muscle lineage. The term encompasses mesoangioblasts, satellite cells, satellite stem cells, muscle satellite stem cells, satellite progenitor cells, muscle satellite progenitor cells, myoblasts, myocytes, Pax7⁺/Myf5⁻/MyoD⁻ cells, Pax7⁺/Myf5⁺/MyoD⁻ cells, and Pax7⁺/Myf5⁺/MyoD⁺ cells.

Self renewal

“Self-renewal” refers to a cell with a unique capacity to produce unaltered daughter cells and to generate specialized cell types (potency). Self-renewal can be achieved in at least two ways. Asymmetric cell division produces one daughter cell that is identical to the parental cell and one daughter cell that is different from the parental cell and is a progenitor or differentiated cell. Asymmetric cell division thus does not increase the number of daughter cells identical to the parental cell, but maintains the number of cells of the parental cell type. Symmetric cell division, in contrast, produces two daughter cells that are each identical to the parental cell. Symmetric cell division thus increases the number of cells identical to the parental cell, expanding the population of parental cells. In particular embodiments, symmetric cell division is used interchangeably with cell expansion, e.g., expansion of the stem cell population

### Differentiation

“Differentiation” refers to a developmental process whereby cells become specialized for a particular function, for example, where cells acquire one or more morphological characteristics and/or functions different from that of the initial cell type. The term “differentiation” includes both lineage commitment and terminal differentiation processes. States of undifferentiation or differentiation may be assessed, for example, by assessing or monitoring the presence or absence of biomarkers using immunohistochemistry or other procedures known to a person skilled in the art.

Lineage commitment

The term “lineage commitment” refers to the process by which a stem cell becomes committed to forming a particular limited range of differentiated cell types. Lineage commitment arises, for example, when a stem cell gives rise to a progenitor cell during asymmetric cell division. Committed progenitor cells are often capable of self-renewal or cell division.

Terminal differentiation

“Terminal differentiation” refers to the final differentiation of a cell into a mature, fully differentiated cell. Usually, terminal differentiation is associated with withdrawal from the cell cycle and cessation of proliferation.

Ex vivo

The term “ex vivo” refers generally to activities that take place outside an organism, such as experimentation or measurements done in or on living tissue in an artificial environment outside the organism, preferably with minimum alteration of the natural conditions. In particular embodiments, “ex vivo” procedures involve living cells or tissues taken from an organism and cultured in a laboratory apparatus, usually under sterile conditions, and typically for a few hours or up to about 24 hours, but including up to 48 or 72 hours, depending on the circumstances.

In vitro

Tissue culture experiments or procedures lasting longer than a few days using living cells or tissue are typically considered to be “in vitro.” In particular embodiments, the term “in vitro” refers to cells or tissues that have been manipulated or cultured for a period of at least 72 hours or more, at least 4, 5, 6, 7 days or more, at least 1, 2, 3, 4 weeks or more, at least 1, 2, 3, 4, 5, 6 months or more, or at least one year or more.

In vivo

The term “in vivo” refers generally to activities that take place inside an organism, such as cell engraftment, cell migration, cell dispersion, cell homing, self-renewal of cells, and expansion of cells.

Therapeutic properties

The term “therapeutic properties” refers to one or more properties in a cell with increased non-canonical Wnt signaling. In particular embodiments, one or more therapeutic properties are increased in a cell after it has been contacted with or cultured in the presence of a non-canonical Wnt signaling pathway activator contemplated herein for a sufficient time to increase non-canonical Wnt signaling in the cell. In one embodiment, a cell with increased non-canonical Wnt signaling acquires one or more of the following therapeutic properties including, but not limited to: increased engraftment potential of the cells, and/or an increased ability of the transplanted cells to disperse from the administration site, to undergo myofusion, to increase force generation, to increase twitch tension; and/or to increase cell motility or cell migration of the cells. A cell comprising an increase in one or more therapeutic properties in a cell as a result of increased non-canonical Wnt signaling may also be referred to as a cell comprising increased therapeutic potential.

Engraftment potential/Engraft

The “engraftment potential” refers to the ability of a cell to engraft. In one embodiment, increased engraftment relates to improved attachment of the cell to a muscle fiber. In particular embodiments, the engraftment potential of a cell, such as a myogenic cell, e.g., a mesoangioblast cell, a satellite cell, satellite stem cell, satellite progenitor cell, myoblast, myocyte, Pax7⁺/Myf5⁻/MyoD⁻ cell, Pax7⁺/Myf5⁺/MyoD⁻ cell, and Pax7⁺/Myf5⁺/MyoD⁺ cell, or cell population comprising any number and combination of the foregoing cell types can be determined by measuring, for example, the activity of non-canonical Wnt signaling pathways, the expression in the cell of genes associated with engraftment, cell viability, and the capacity of the cell to self-renew. In particular embodiments, a cell comprising increased non-canonical Wnt signaling comprises increased engraftment potential as a function of the cell's increased dispersion and motility and myofusion. Of course, the skilled artisan would appreciate other suitable assays that would also indicate an increased engraftment potential in a myogenic cell. As used herein, the term “engraft” refers to the ability of a cell to integrate into a location, such as a tissue, e.g., cardiac or skeletal muscle tissue, and persist in the particular location over time.

### Hypertrophy

“Muscle hypertrophy” refers to an increase in muscle size, and may include an increase in individual fiber volume and/or an increase in the cross-sectional area of myofibers, and may also include an increase in the number of nuclei per muscle fiber. Muscle hypertrophy may also include an increase in the volume and mass of whole muscles; however, muscle hypertrophy can be differentiated from muscle hyperplasia, which is the formation of new muscle cells. In one embodiment, muscular hypertrophy refers to an increase in the number of actin and myosin contractile proteins.

Cell motility or cell migration

“Cell motility” or “cell migration” refers to the ability of a transplanted cell to move away from the point of transplant. Cell motility or migration may be influenced by secretion of guidance factors from resident cells or tissues as well as expression of guidance factor gradient sensing molecules in the transplanted cells. In one embodiment, the movement of cells away from the site of transplant, e.g., administration or injection site, is known as dispersion.

### Increase

As used herein, the terms “promoting,” “enhancing,” “stimulating,” or “increasing” generally refer to the ability of a cell comprising increased non-canonical Wnt signaling or another composition contemplated herein to produce or cause a greater physiological response (i.e., measurable downstream effect), as compared to the response caused by either vehicle or a control molecule/composition. One such measurable physiological response includes, without limitation, increased engraftment, increased engraftment potential, increased dispersion of transplanted cells, increased myofusion, increased force generation, increased twitch tension, increased motility, increased migration or any combination thereof compared to normal, untreated, or control-treated cells. The physiological response may be increased by at least 5%, 10%, 20%, 30%, 40%, 50%, 60%, 70%, 80%, 90%, 100%, 125%, 150%, 175%, 200%, or greater compared to the response measured in normal, untreated, or control-treated cells. An “increased” or “enhanced” response or property is typically “statistically significant”, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) that produced by normal, untreated, or control-treated cells.

### Maintain

As used herein, the terms “retaining” or “maintaining,” generally refer to the ability of a cell comprising increased non-canonical Wnt signaling or another composition contemplated herein to produce or cause a physiological response (i.e., measurable downstream effect) that is of a similar nature or that is substantially the same as a response caused by normal, untreated, or control-treated cells.

### Decrease

As used herein, the terms “decrease” or “lower,” or “lessen,” or “reduce,” or “abate” refers generally to the ability of a cell comprising increased non-canonical Wnt signaling or another composition contemplated herein to produce or cause a lesser physiological response (i.e., downstream effects), as compared to the response caused by either vehicle or a control molecule/composition, e.g., decreased apoptosis. In one embodiment, the decrease can be a decrease in gene expression or a decrease in cell signaling that normally is associated with a reduction of cell viability. A “decrease” or “reduced” response is typically a “statistically significant” response, and may include an decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response produced by normal, untreated, or control-treated cells.

Conditions sufficient

As used herein, the terms “conditions sufficient,” or “under conditions sufficient,” refer to the incubation or culture conditions for treating the source of transplant material, for example, stem or progenitor cells, and/or other populations of cells comprising satellite stem and/or progenitor cells, and/or populations of cells comprising Pax7⁺/Myf5⁻/MyoD⁻, Pax7⁺/Myf5⁺/MyoD⁻, Pax7⁺/Myf5⁺/MyoD⁺ and/or other myogenic cells, with one or more non-canonical Wnt signaling pathway activators. In one embodiment, “conditions sufficient” include contacting the cells with a non-canonical Wnt signaling pathway activator and/or activating or increasing non-canonical Wnt signaling in a cell for sufficient time or duration. In one embodiment, “conditions sufficient” include contacting the cells with a sufficient amount, e.g., an effective amount, of a non-canonical Wnt signaling pathway activator, sufficient to activate or increase non-canonical Wnt signaling in the cell. In a particular embodiment, the conditions are sufficient to increase engraftment, engraftment potential, dispersion, myofusion, force generation, twitch tension, motility, migration, or any combination thereof in a cell-based therapy contemplated herein that is administered to a subject.

Gene therapy

As used herein, the term “gene therapy” refers to the introduction of a polynucleotide into a cell that restores, corrects, or modifies the gene and/or expression of the gene. In particular embodiments, the polynucleotide is incorporated into the cell's genome and in other embodiments, the polynucleotide is episomal.

Therapeutic polypeptide

In various embodiments, a cell is genetically modified to express a “therapeutic polypeptide.” As used herein, the term “therapeutic polypeptide” refers to a peptide that confers at least one ameliorative, preventative, or therapeutic benefit when expressed in a subject. In one embodiment, a therapeutic polypeptide is delivered to a subject by fusion of a myogenic cell with the muscle tissue of the subject to form a multinucleate syncytium.

Polynucleotide-of-interest

The term “polynucleotide-of-interest” refers to a polynucleotide encoding a therapeutic polypeptide or polypeptide-of-interest.

Polypeptide-of-interest

The term “polypeptide-of-interest” refers to a polypeptide for which expression in a cell contemplated herein is desired. In one embodiment, the term “polypeptide-of-interest” is used interchangeably with the term “therapeutic polypeptide.” In other embodiments, the term “polypeptide-of-interest” refers to a polypeptide that provides at least one ameliorative or preventative benefit when expressed in a subject, but that may not provide a therapeutically relevant benefit.

### Subject

A “subject,” “subject in need of treatment,” “subject in need thereof,” “individual,” or “patient” as used herein, includes as used herein, includes any animal that exhibits a symptom of a disease, disorder, or condition that can be treated with the cell-based therapies contemplated herein. The disorder may be monogenetic, polygenetic, and/or a progressive and/or degenerative disease, disorder, or condition. In preferred embodiments, a subject includes any animal that exhibits symptoms of a degenerative disease, disorder, or condition of the nervous system or musculoskeletal system that can be treated with the cell-based therapeutics and methods contemplated herein. Suitable subjects include laboratory animals (such as mouse, rat, rabbit, or guinea pig), farm animals (such as horses, cows, sheep, pigs), and domestic animals or pets (such as a cat or dog). In particular embodiments, the subject is a mammal. In certain embodiments, the subject is a non-human primate and, in preferred embodiments, the subject is a human. Typical subjects include animals that exhibit aberrant amounts (lower or higher amounts than a “normal” or “healthy” subject) of one or more physiological activities that can be modulated by the cell-based therapies contemplated herein.

### Treatment

As used herein “treatment” or “treating,” includes any beneficial or desirable effect on the symptoms or pathology of a disease or pathological condition, and may include even minimal reductions in one or more measurable markers of the disease or condition being treated. Treatment can involve optionally either the reduction or amelioration of symptoms of the disease or condition, or the delaying of the progression of the disease or condition. “Treatment” does not necessarily indicate complete eradication or cure of the disease or condition, or associated symptoms thereof.

### Prevention

As used herein, “prevent,” and similar words such as “prevented,” “preventing” etc., indicate an approach for preventing, inhibiting, or reducing the likelihood of the occurrence or recurrence of, a disease or condition. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

### Amount

As used herein, the term “amount” refers to “an amount effective” or “an effective amount” of cells sufficient to achieve a beneficial or desired prophylactic or therapeutic result, including clinical results. In one embodiment an effect amount refers to the amount of a non-canonical Wnt signaling pathway activator to increase non-canonical Wnt signaling in a cell.

Prophylactic amount

A “prophylactically effective amount” refers to an amount of cells effective to achieve the desired prophylactic result. Typically but not necessarily, since a prophylactic dose is used in subjects prior to or at an earlier stage of disease, the prophylactically effective amount is less than the therapeutically effective amount.

Therapeutically effective

A “therapeutically effective amount” of cells may vary according to factors such as the disease state, age, sex, and weight of the individual, and the ability of the stem and progenitor cells to elicit a desired response in the individual. A therapeutically effective amount is also one in which any toxic or detrimental effects of the therapeutic cells are outweighed by the therapeutically beneficial effects. The term “therapeutically effective amount” includes an amount that is effective to “treat” a subject (e.g., a patient).

### C. Non-Canonical Wnt Signaling Pathway Activators

In various embodiments, one or more cells are contacted with or cultured in the presence of a non-canonical Wnt signaling pathway activator and/or are modified to express one or more non-canonical Wnt signaling pathway activators. As used herein, the terms “non-canonical Wnt signaling pathway activator” and “non-canonical Wnt signaling activator” are used interchangeably and refer to a small molecule, polypeptide, or polynucleotide that increases signal transduction through the non-canonical Wnt signaling pathway. In preferred embodiments, the non-canonical Wnt signaling activator increases Wnt7a/Fzd7 signaling.

The non-canonical Wnt pathway is often referred to as the β-catenin-independent pathway and, while not as well-defined as the canonical pathway, this pathway can be further divided into at least two distinct branches, the Planar Cell Polarity pathway (or PCP pathway) and the Wnt/Ca²⁺ pathway. The PCP pathway emerged from genetic studies in Drosophila in which mutations in Wnt signaling components including Frizzled (Fzd) and Dishevelled (Dsh) were found to randomize the orientation of epithelial structures including cuticle hairs and sensory bristles. Non-canonical Wnt signaling is transduced through Fzd independent of LRP5/6 leading to the activation of Dsh. Dsh through Daam1 mediates activation of Rho which in turn activates Rho kinase (Rock). Daam1 also mediates actin polymerization through the actin binding protein Profilin. Dsh also mediates activation of Rac, which in turn activates JNK. The signaling from Rock, JNK and Profilin are integrated for cytoskeletal changes for cell polarization and motility during gastrulation.

1. Small Molecule Activators of Non-Canonical Wnt Signaling

In particular embodiments, a cell or population of cells is contacted with one or more small molecule activators of non-canonical Wnt signaling. In one embodiment, one or more cells are contacted with an effective amount of a small molecule activator of non-canonical Wnt signaling for a time sufficient to increase non-canonical Wnt signaling and to increase one or more therapeutic properties of the cell, e.g., increased engraftment potential.

The term “small molecule activator of non-canonical Wnt signaling” refers to small molecules that can increase non-canonical Wnt signaling, e.g., Wn7a/Fzd7 signaling, in a cell, either alone or in combination with other factors. A “small molecule” refers to an agent that has a molecular weight of less than about 5 kD, less than about 4 kD, less than about 3 kD, less than about 2 kD, less than about 1 kD, or less than about 0.5 kD. Small molecules include, but are not limited to: nucleic acids, peptidomimetics, peptoids, carbohydrates, lipids or other organic or inorganic molecules. Libraries of chemical and/or biological mixtures, such as fungal, bacterial, or algal extracts, are known in the art and can be used as a source of small molecules in certain embodiments. In particular embodiments, the small molecule reprogramming agent used herein has a molecular weight of less than 10,000 daltons, for example, less than 8000, 6000, 4000, 2000 daltons, e.g., between 50-1500, 500-1500, 200-2000, 500-5000 daltons.

Illustrative examples of small molecule activators of non-canonical Wnt signaling include, but are not limited to, small molecule activators of Akt/mTOR, Dvl2, Rac1 GTPases, RhoA GTPases, small GTPases, and cdc42, and their downstream effectors.

2. Polypeptides that Activate Non-Canonical Wnt Signaling

In particular embodiments, one or more cells are contacted with a polypeptide that activates non-canonical Wnt signaling or, a polypeptide that activates non-canonical Wnt signaling is introduced into the cell. In one embodiment, one or more cells are contacted with an effective amount of a polypeptide that activates non-canonical Wnt signaling for a time sufficient to increase non-canonical Wnt signaling and to increase one or more therapeutic properties of the cell, e.g., increased engraftment potential.

“Polypeptide,” “polypeptide fragment,” “peptide” and “protein” are used interchangeably and according to conventional meaning, i.e., a sequence of amino acids, unless specified to the contrary. In one embodiment, a “polypeptide” includes homologs, paralogs, or orthologs, polypeptides comprising one or more modifications, and fusion polypeptides. Polypeptides can be prepared using any of a variety of well known recombinant and/or synthetic techniques. Polypeptides are not limited to a specific length, e.g., they may comprise a full length protein sequence, a fragment of a full length protein, or a fusion protein, and may include or lack one or more post-translational modifications of the polypeptide, for example, glycosylations, acetylations, phosphorylations, lipidations, and the like, as well as other modifications known in the art, both naturally occurring and non-naturally occurring.

An “isolated polypeptide” refers to the in vitro isolation and/or purification of a polypeptide molecule from a cellular environment, and from association with other components of the cell, i.e., it is not significantly associated with in vivo substances. In preferred embodiments, isolated polypeptides contemplated herein are recombinant and comprise one or more non-naturally occurring modifications.

As used herein, the term “naturally occurring”, refers to a polypeptide or polynucleotide sequence that can be found in nature. For example, a naturally occurring polypeptide or polynucleotide sequence would be one that is present in an organism, and can be isolated from the organism, and which has not been intentionally modified by man in the laboratory. The term “wild-type” is often used interchangeably with the term “naturally occurring.”

In particular embodiments, a polypeptide that activates non-canonical Wnt signaling comprises a non-canonical Wnt polypeptide or a modified non-canonical Wnt polypeptide. Modifications include, but not limited to truncations, biologically active fragments, variants, and fusion polypeptides as contemplated herein. The term “non-canonical Wnt polypeptide,” refers to a Wnt polypeptide that generally or predominantly signals through non-canonical Wnt signaling pathways. Exemplary non-canonical Wnt polypeptides include, but are not limited to Wnt4, Wnt5a, Wnt5b, Wnt6, Wnt7a, Wnt7b, and Wnt11. In particular embodiments, the term “non-canonical Wnt polypeptide,” refers to a recombinant and/or modified non-canonical Wnt polypeptide having a sequence that is at least about 70%, more preferably about 80%, 85%, 90%, 91%, 92%, 93%, 94%, 95%, 96%, 97%, 98%, 99% or about 100%, identical to a naturally occurring non-canonical Wnt polypeptide sequence. Identity may be assessed over at least about 10, 25, 50, 100, 200, 300, or more contiguous amino acids, or may be assessed over the full length of the sequence. Methods for determining % identity or % homology are known in the art and any suitable method may be employed for this purpose. Illustrative examples of Wnt polypeptides are set forth in U.S. Ser. No. 13/979,368, filed Jan. 11, 2012, U.S. Ser. No. 14/344,310, filed Sep. 14, 2012, and U.S. Ser. No. 14/344,39, filed Sep. 14, 2012, the disclosures, descriptions, and sequences of which are incorporated by reference herein in their entirety.

Additional illustrative examples of Wnt polypeptides are set forth in SEQ ID NOs: 3-56 and 64-77.

Activity of non-canonical Wnt polypeptides or modified non-canonical Wnt polypeptides can be determined by measuring the ability to mimic wild-type Wnt biological activity and comparing the ability to the activity of a wild type protein.

In particular embodiments, the non-canonical Wnt is selected from the group consisting of: Wnt4, Wnt5a, Wnt5b, Wnt6, Wnt7a, Wnt7b, and Wnt11. In preferred embodiments, the non-canonical Wnt is Wnt5a or Wnt7a. In particular preferred embodiments, the non-canonical Wnt is Wnt7a, optionally human Wnt7a.

In particular embodiments, a polypeptide that activates non-canonical Wnt signaling comprises a Fzd polypeptide or a modified Fzd polypeptide. Modifications include, but not limited to truncations, biologically active fragments, variants, and fusion polypeptides as contemplated herein. The terms “Fzd”, “Fzd polypeptides” and “Fzd receptors” are used interchangeably and to refer to proteins of the Frizzled receptor family. These proteins are seven-pass transmembrane proteins (Ingham, P. W. (1996) Trends Genet. 12: 382-384; Yang-Snyder, J. et al. (1996) Curr. Biol. 6: 1302-1306; Bhanot, P. et al. (1996) Nature 382: 225-230) that comprise a CRD domain. There are ten known members of the Fzd family (Fzd1 through Fzd10), any of which may server as receptors of Wnts. Exemplary Fzd polypeptides include, but are not limited to, Fzd1, Fzd2, Fzd3, Fzd4, Fzd5, Fzd6, Fzd7, Fzd8, Fzd9, and Fzd10.

In particular embodiments, the term “Fzd polypeptide,” refers to recombinant and/or modified Fzd polypeptide having a sequence that is at least about 70%, more preferably about 80%, 85%, 90%, 91%, 92%, 93%, 94%, 95%, 96%, 97%, 98%, 99% or about 100%, identical to a naturally occurring Fzd polypeptide sequence. Identity may be assessed over at least about 10, 25, 50, 100, 200, 300, or more contiguous amino acids, or may be assessed over the full length of the sequence. Methods for determining % identity or % homology are known in the art and any suitable method may be employed for this purpose. Illustrative examples of Fzd polypeptides are set forth in SEQ ID NOs: 78-87. Activity of a Fzd polypeptide or a modified Fzd polypeptide can be determined by measuring its ability to mimic wild-type Fzd biological activity and comparing the ability to the activity of a wild type protein.

In particular embodiments, the Fzd polypeptide is selected from the group consisting of: Fzd1, Fzd2, Fzd3, Fzd4, Fzd5, Fzd6, Fzd7, Fzd8, Fzd9, and Fzd10. In preferred embodiments, the Fzd polypeptide is Fzd7, optionally human Fzd7.

In various embodiments, modified polypeptides that activate non-canonical Wnt signaling include, but are not limited to truncated polypeptides, biologically active polypeptide fragments, polypeptide variants, and fusion polypeptides, are contemplated. In preferred embodiments, the modified polypeptide is a non-canonical Wnt polypeptide, e.g., Wnt7a, or a Fzd polypeptide, e.g., Fzd7.

In particular embodiments, modified polypeptides that activate non-canonical Wnt signaling have been modified or engineered to comprise an N-terminal and/or C-terminal deletion or truncation of one or more amino acid residues, but retain non-canonical Wnt signaling activity. In particular embodiments, truncated polypeptides retain at least 100%, at least 90%, at least 80%, at least 70%, at least 60%, at least 50%, at least 40%, at least 30%, at least 20%, at least 10%, or at least 5% of the naturally occurring polypeptide activity.

In particular embodiments, a truncated polypeptide is a truncated Wnt polypeptide, e.g., a Wnt7a polypeptide comprising an N-terminal deletion or truncation of at least 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 217, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, or 300 N-terminal amino acids. In particular embodiments, a Wnt polypeptide according to the invention, comprises an N-terminal deletion or truncation sufficient to eliminate one or more Wnt lipidation sites. In a certain embodiment, a Wnt polypeptide comprises an N-terminal deletion of at least 50, 60, 70, 80, 90, 100, 110, 120, 130, 140, 150, 160, 170, 180, 190, 200, 210, 220, 230, 240, 250, 260, 270, 280, 290 or 300 N-terminal amino acids. In particular embodiments, a truncated polypeptide is a truncated Wnt7a polypeptide comprising a C-terminal deletion or truncation of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 C-terminal amino acids. In certain embodiments, truncated non-canonical Wnt polypeptides comprise an N-terminal deletion or truncation of about 220 to about 284 N-terminal amino acids and a C-terminal deletion or truncation of about 1 to about 50 C-terminal amino acids.

Illustrative examples of truncated Wnt polypeptides are disclosed in U.S. Ser. No. 14/344,310, filed Sep. 14, 2012, and U.S. Ser. No. 14/344,39, filed Sep. 14, 2012, the disclosures, descriptions, and sequences of which are incorporated by reference herein in their entirety.

Additional illustrative examples of truncated Wnt polypeptides are set forth in SEQ ID NOs: 38-56 and 64-77.

In certain embodiments, modified polypeptides that activate non-canonical Wnt signaling include a minimal biologically active fragment of a polypeptide comprising one or more N-terminal amino acid truncations and one or more C-terminal amino acid truncations as described elsewhere herein. As used herein, the term “minimal active fragment” or “minimal biologically active fragment” refers to a polypeptide fragment that retains at least 100%, at least 90%, at least 80%, at least 70%, at least 60%, at least 50%, at least 40%, at least 30%, at least 20%, at least 10%, or at least 5% of the naturally occurring Wnt polypeptide activity. In particular embodiments, the present invention contemplates, minimal biologically active fragments comprising 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118 119, 120, 121, 122, 123, 124, 125, 126, 127, 128, or 129 contiguous amino acids of polypeptide.

In preferred embodiments, a minimal biologically active fragment of a non-canonical Wnt, e.g., Wnt7a is provided comprising 30, 35, 40, 45, 50, 55, 60, 0, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150 contiguous amino acids including all integers (e.g., 101, 102, 103) and ranges (e.g., 50-75, 75-100, 100-129) in between, of the amino acid sequences set forth in any one of the Wnt polypeptides described herein or disclosed in U.S. Ser. No. 14/344,310, filed Sep. 14, 2012, and U.S. Ser. No. 14/344,39, filed Sep. 14, 2012, the disclosures, descriptions, and sequences of which are incorporated by reference herein in their entirety.

In particular embodiments, modified polypeptides that activate non-canonical Wnt signaling include polypeptide variants. The term “variant” refers to polypeptides that are distinguished from a reference polypeptide by the modification, addition, deletion, or substitution of at least one amino acid residue. In certain embodiments, a polypeptide variant is distinguished from a reference polypeptide by one or more amino acid substitutions (e.g., 1, 2, 3, 4, 5 or more substitutions), which may be conservative or non-conservative. In certain embodiments, polypeptide variants comprise one or more amino acid additions, deletions, or substitutions in order to prevent lipidation, to increase non-canonical Wnt signaling activity, and/or to increase stability of the modified polypeptide compared to the naturally occurring polypeptide. In particular embodiments, non-canonical Wnt polypeptide variants comprise one or more amino acid additions, deletions, or substitutions in order to prevent lipidation, to increase non-canonical Wnt pathway signaling activity, and/or to increase stability of the modified non-canonical Wnt polypeptide compared to the naturally occurring non-canonical polypeptide.

In other particular embodiments, a non-canonical Wnt polypeptide variant comprises an amino acid mutation, addition, deletion, and/or substitution at one or more of the amino acid positions identified in U.S. Ser. No. 13/979,368, filed Jan. 11, 2012, U.S. Ser. No. 14/344,310, filed Sep. 14, 2012, and U.S. Ser. No. 14/344,39, filed Sep. 14, 2012, the disclosures, descriptions, and sequences of which are incorporated by reference herein in their entirety.

In particular embodiments, the non-canonical Wnt polypeptide variant is a Wnt7a variant comprising an amino acid mutation, addition, deletion, and/or substitution at amino 206 that prevents lipidation at such position(s), wherein the Wnt7a polypeptide retains or has increased levels of Wnt7a biological activity, e.g., SEQ ID NO: 3. In certain embodiments, S206 of a Wnt7a polypeptide is substituted with Ala or another amino acid that prevents lipidation of these residues. Such Wnts and modifications are disclosed in related applications U.S. Ser. No. 13/979,368, filed Jan. 11, 2012, U.S. Ser. No. 14/344,310, filed Sep. 14, 2012, and U.S. Ser. No. 14/344,39, filed Sep. 14, 2012, the disclosures, descriptions, and sequences of which are incorporated by reference herein in their entirety.

In particular embodiments, modified polypeptides that activate non-canonical Wnt signaling include fusion polypeptides. Fusion polypeptides contemplated herein may comprise a signal peptide at the N-terminal end, which co-translationally or post-translationally directs transfer of the protein; a truncated, biologically active fragment, or variant polypeptide as contemplated herein, e.g., a non-canonical Wnt polypeptide (e.g., Wnt7a) of Fzd polypeptide (e.g., Fzd7). Fusion polypeptides may also comprise linkers or spacers, Fc domains, one or more protease cleavage sites, or one or more epitope tags or other sequence for ease of synthesis, purification or production of the polypeptide. Fusion polypeptide and fusion proteins refer to a polypeptide of the invention that has been covalently linked, either directly or via an amino acid linker, to one or more heterologous polypeptide sequences (fusion partners), including, but not limited to cell permeable peptides. The polypeptides forming the fusion protein are typically linked C-terminus to N-terminus, although they can also be linked C-terminus to C-terminus, N-terminus to N-terminus, or N-terminus to C-terminus. The polypeptides of the fusion protein can be in any order.

As used herein, the term “signal peptide” refers to a leader sequence ensuring entry into the secretory pathway. For industrial production of a secreted protein, the protein to be produced needs to be secreted efficiently from the host cell or the host organism. The signal peptide may be, e.g., the native signal peptide of the protein to be produced, a heterologous signal peptide, or a hybrid of native and heterologous signal peptide. Numerous signal peptides are used for production of secreted proteins.

Illustrative examples of signal peptides for use in fusion polypeptides of the invention include, but are not limited to: a CD33 signal peptide; an immunoglobulin signal peptide, e.g., an IgGκ signal peptide or an IgGμ signal peptide; a growth hormone signal peptide; an erythropoietin signal peptide; an albumin signal peptide; a secreted alkaline phosphatase signal peptide, and a viral signal peptide, e.g., rotovirus VP7 glycoprotein signal peptide.

In particular embodiments, the fusion polypeptides comprise protease cleavage sites and epitope tags to facilitate purification and production of polypeptides. The position of the protease cleavage site is typically between the C-terminus of the Wnt polypeptide and the epitope tag to facilitate removal of heterologous sequences prior to delivery of the polypeptide to a cell or tissue.

Illustrative examples of heterologous protease cleavage sites that can be used in fusion proteins of the invention include, but are not limited to: a tobacco etch virus (TEV) protease cleavage site, a heparin cleavage site, a thrombin cleavage site, an enterokinase cleavage site and a Factor Xa cleavage site.

Illustrative examples of epitope tags that can be used in fusion proteins of the invention include, but are not limited to: a HIS6 epitope, a MYC epitope, a FLAG epitope, a V5 epitope, a VSV-G epitope, and an HA epitope.

A peptide linker sequence may also be employed to separate the fusion polypeptide components by a distance sufficient to ensure that each polypeptide folds into its secondary and tertiary structures, if desired. Such a peptide linker sequence is incorporated into the fusion protein using standard techniques well known in the art. Amino acid sequences which may be usefully employed as linkers include those disclosed in Maratea et al., Gene 40:39 46 (1985); Murphy et al., Proc. Natl. Acad. Sci. USA 83:8258 8262 (1986); U.S. Pat. No. 4,935,233 and U.S. Pat. No. 4,751,180. Other linkers that may be used include Glu-Gly-Lys-Ser-Ser-Gly-Ser-Gly-Ser-Glu-Ser-Lys-Val-Asp (SEQ ID NO:110) (Chaudhary et al., 1990, Proc. Natl. Acad. Sci. U.S.A. 87:1066-1070) and Lys-Glu-Ser-Gly-Ser-Val-Ser-Ser-Glu-Gln-Leu-Ala-Gln-Phe-Arg-Ser-Leu-Asp (SEQ ID NO:111) (Bird et al., 1988, Science 242:423-426).

In various embodiments, fusions polypeptides comprising a truncated polypeptide and an Fc domain are provided. The Fc-domain can be fused to the N-terminus or C-terminus of the polypeptide. The Fc domain can be obtained from any of the classes of immunoglobulin, IgG, IgA, IgM, IgD and IgE. In some embodiments, the Fc region is a wild-type Fc region. In some embodiments, the Fc region is a mutated Fc region. In some embodiments, the Fc region is truncated at the N-terminal end by 1, 2, 3, 4, 5, 6, 7, 8, 9, or 10 amino acids, (e.g., in the hinge domain).

In various embodiments, a fusion polypeptide comprises a polypeptide that increases non-canonical Wnt signaling fused to one or more cell permeable peptides (CPP). Illustrative examples of peptide sequences which can facilitate protein uptake into cells include, but are not limited to: HIV TAT polypeptides; a 20 residue peptide sequence which corresponds to amino acids 84-103 of the p16 protein (see Fahraeus et al., 1996. Curr. Biol. 6:84); the third helix of the 60-amino acid long homeodomain of Antennapedia (Derossi et al., 1994. J. Biol. Chem. 269:10444); the h region of a signal peptide, such as the Kaposi fibroblast growth factor (K-FGF) h region; and the VP22 translocation domain from HSV (Elliot et al., 1997. Cell 88:223-233). In addition, Several bacterial toxins, including Clostridium perfringens iota toxin, diphtheria toxin (DT), Pseudomonas exotoxin A (PE), Bordetella pertussis toxin (PT), Bacillus anthracis toxin, and Bordetella pertussis adenylate cyclase (CYA), have been used to deliver peptides to the cell cytosol as internal or amino-terminal fusions. Arora et al., 1993. J. Biol. Chem. 268:3334-3341; Perelle et al., 1993. Infect. Immun. 61:5147-5156; Stenmark et al., 1991. J. Cell Biol. 113:1025-1032; Donnelly et al., 1993. Proc. Natl. Acad. Sci. USA 90:3530-3534; Carbonetti et al., 1995. Abstr. Annu. Meet. Am. Soc. Microbiol. 95:295; Sebo et al., 1995. Infect. Immun. 63:3851-3857; Klimpel et al., 1992. Proc. Natl. Acad. Sci. USA. 89:10277-10281; and Novak et al., 1992. J. Biol. Chem. 267:17186-17193.

Other exemplary CPP amino acid sequences include, but are not limited to: RKKRRQRRR (SEQ ID NO:100), KKRRQRRR (SEQ ID NO:101), and RKKRRQRR (SEQ ID NO:102) (derived from HIV TAT protein); RRRRRRRRR (SEQ ID NO:103); KKK (SEQ ID NO:104); RQIKIWFQNRRMKWKK (SEQ ID NO: 105) (from Drosophila Antp protein); RQIKIWFQNRRMKSKK (SEQ ID NO:106) (from Drosophila Ft protein); RQIKIWFQNKRAKIKK (SEQ ID NO:107) (from Drosophila Engrailed protein); RQIKIWFQNRRMKWKK (SEQ ID NO:108) (from human Hox-A5 protein); and RVIRVWFQNKRCKDKK (SEQ ID NO:109) (from human Isl-1 protein). Such subsequences can be used to facilitate polypeptide translocation, including the fusion polypeptides contemplated herein, across a cell membrane.

3. Polynucleotides that Activate Non-Canonical Wnt Signaling

In particular embodiments, an exogenous polynucleotide that activates non-canonical Wnt signaling is introduced into one or more cells to express a polypeptide that activates non-canonical Wnt signaling. In one embodiment, one or more cells are modified by the introduction of an exogenous polynucleotide, to express an effective amount of a polypeptide, encoded by the polynucleotide, that activates non-canonical Wnt signaling for a time sufficient to increase non-canonical Wnt signaling and to increase one or more therapeutic properties of the cell, e.g., increased engraftment potential. In various embodiments, polynucleotides encoding polypeptides that activate non-canonical Wnt signaling are contemplated.

As used herein, the term “gene” may refer to a synthetic and/or chimeric non-naturally occurring polynucleotide sequence comprising enhancers, promoters, introns, exons, and the like. In particular embodiments, the term “gene” refers to a polynucleotide sequence encoding a polypeptide, regardless of whether the polynucleotide sequence is identical to the genomic sequence encoding the polypeptide.

An “isolated polynucleotide,” as used herein, refers to a polynucleotide that has been purified from the sequences which flank it in a naturally-occurring state, e.g., a DNA fragment that has been removed from the sequences that are normally adjacent to the fragment, a synthetic polynucleotide and/or chimeric non-naturally occurring polynucleotide. In particular embodiments, an “isolated polynucleotide” refers to a complementary DNA (cDNA), a recombinant DNA, or other synthetic polynucleotide that does not exist in nature and that has been made by the hand of man.

In particular embodiments, one or more polynucleotides may be arranged in any suitable order within a larger polynucleotide, such as a vector. In preferred embodiments, the vector that integrates into the genome of a host cell including, but not limited to, a retroviral, e.g., lentiviral vector. In other preferred embodiments, the vector is an episomal vector.

The polynucleotides contemplated herein, regardless of the length of the coding sequence itself, may be combined with other DNA sequences, such as expression control sequences, promoters and/or enhancers, untranslated regions (UTRs), Kozak sequences, polyadenylation signals, additional restriction enzyme sites, multiple cloning sites, internal ribosomal entry sites (IRES), recombinase recognition sites (e.g., LoxP, FRT, and Att sites), termination codons, transcriptional termination signals, and polynucleotides encoding self-cleaving polypeptides, epitope tags, as disclosed elsewhere herein or as known in the art, such that their overall length may vary considerably. It is therefore contemplated that a polynucleotide fragment of almost any length may be employed, with the total length preferably being limited by the ease of preparation and use in the intended recombinant DNA protocol.

Polynucleotides can be prepared, manipulated and/or expressed using any of a variety of well established techniques known and available in the art. In order to express a desired polypeptide, a nucleotide sequence encoding the polypeptide, can be inserted into appropriate vector. Examples of vectors are plasmid, autonomously replicating sequences, and transposable elements. Additional exemplary vectors include, without limitation, plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Examples of categories of animal viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40). Examples of expression vectors are pClneo vectors (Promega) for expression in mammalian cells; pLenti4/V5-DEST™, pLenti6/V5-DEST™, and pLenti6.2/V5-GW/lacZ (Invitrogen) for lentivirus-mediated gene transfer and expression in mammalian cells. In particular embodiments, coding sequences of polypeptides disclosed herein can be ligated into such expression vectors for the expression of the polypeptides in mammalian cells.

In particular embodiments, the vector is an episomal vector or a vector that is maintained extrachromosomally. As used herein, the term “episomal” refers to a vector that is able to replicate without integration into host's chromosomal DNA and without gradual loss from a dividing host cell also meaning that said vector replicates extrachromosomally or episomally. The vector is engineered to harbor the sequence coding for the origin of DNA replication or “ori” from a lymphotrophic herpes virus or a gamma herpesvirus, an adenovirus, SV40, a bovine papilloma virus, or a yeast, specifically a replication origin of a lymphotrophic herpes virus or a gamma herpesvirus corresponding to oriP of EBV. In a particular aspect, the lymphotrophic herpes virus may be Epstein Barr virus (EBV), Kaposi's sarcoma herpes virus (KSHV), Herpes virus saimiri (HS), or Marek's disease virus (MDV). Epstein Barr virus (EBV) and Kaposi's sarcoma herpes virus (KSHV) are also examples of a gamma herpesvirus. Typically, the host cell comprises the viral replication transactivator protein that activates the replication.

“Expression control sequences,” “control elements,” or “regulatory sequences” present in an expression vector are those non-translated regions of the vector—origin of replication, selection cassettes, promoters, enhancers, translation initiation signals (Shine Dalgarno sequence or Kozak sequence) introns, a polyadenylation sequence, 5′ and 3′ untranslated regions—which interact with host cellular proteins to carry out transcription and translation. Such elements may vary in their strength and specificity. Depending on the vector system and host utilized, any number of suitable transcription and translation elements, including ubiquitous promoters and inducible promoters may be used.

The term “operably linked” refers to a juxtaposition wherein the components described are in a relationship permitting them to function in their intended manner. In one embodiment, the term refers to a functional linkage between an expression control sequence (such as a promoter, and/or enhancer) and a second polynucleotide sequence, wherein the expression control sequence directs transcription of the nucleic acid corresponding to the second sequence.

Illustrative ubiquitous expression control sequences suitable for use in particular embodiments of the invention include, but are not limited to, a cytomegalovirus (CMV) immediate early promoter, a viral simian virus 40 (SV40) (e.g., early or late), a Moloney murine leukemia virus (MoMLV) LTR promoter, a Rous sarcoma virus (RSV) LTR, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus (Irions et al., Nature Biotechnology 25, 1477-1482 (2007)), a Ubiquitin C promoter (UBC), a phosphoglycerate kinase-1 (PGK) promoter, a cytomegalovirus enhancer/chicken β-actin (CAG) promoter, and a β-actin promoter.

Illustrative examples of inducible promoters/systems include, but are not limited to, steroid-inducible promoters such as promoters for genes encoding glucocorticoid or estrogen receptors (inducible by treatment with the corresponding hormone), metallothionine promoter (inducible by treatment with various heavy metals), MX-1 promoter (inducible by interferon), the “GeneSwitch” mifepristone-regulatable system (Sirin et al., 2003, Gene, 323:67), the cumate inducible gene switch (WO 2002/088346), tetracycline-dependent regulatory systems, etc.

Conditional expression can also be achieved by using a site specific DNA recombinase. According to certain embodiments of the invention, polynucleotides comprises at least one (typically two) site(s) for recombination mediated by a site specific recombinase. As used herein, the terms “recombinase” or “site specific recombinase” include excisive or integrative proteins, enzymes, co-factors or associated proteins that are involved in recombination reactions involving one or more recombination sites (e.g., two, three, four, five, six, seven, eight, nine, ten or more), which may be wild-type proteins (see Landy, Current Opinion in Biotechnology 3:699-707 (1993)), or mutants, derivatives (e.g., fusion proteins containing the recombination protein sequences or fragments thereof), fragments, and variants thereof. Illustrative examples of recombinases suitable for use in particular embodiments of the present invention include, but are not limited to: Cre, Int, IHF, Xis, Flp, Fis, Hin, Gin, ΦC31, Cin, Tn3 resolvase, TndX, XerC, XerD, TnpX, Hjc, Gin, SpCCE1, and ParA.

### D. Cell-Based Compositions

In various embodiments, improved cell-based compositions having one or more therapeutic properties are provided. These cells are otherwise referred to as therapeutic cells, therapeutic cellular compositions, therapeutic compositions, and equivalents. The cells contemplated herein comprise increased non-canonical Wnt signaling, which in turn increases cell motility, migration, dispersion, engraftment potential, and/or engraftment. The cell compositions are useful for providing regenerative therapy and/or gene therapy. In particular embodiments, the cells are genetically modified to provide cell-based gene therapy to a subject in need thereof.

1. Starting Populations of Cells

A starting population of cells suitable for use in particular embodiments may be derived from essentially any suitable source, and may be heterogeneous or homogeneous with respect to cell types and may comprise stem cells, progenitor cells, and/or differentiated cells. Suitable cells include both fetal cells and adult cells. In addition, suitable cells may be mammalian in origin, and in preferred embodiments, human cells. In particular embodiments, the population of cells does not comprise hematopoietic cells.

The cells may be somatic, non-pluripotent, incompletely or partially pluripotent stem cells, multipotent stem cells, oligopotent stem cells, unipotent stem cells, progenitor cells, terminally differentiated cells, or a mixed population of cells comprising any combination of the foregoing. Pluripotent cells suitable for use in particular embodiments include, but are not limited to, naturally-occurring stem cells, embryonic stem cells, or induced pluripotent stem cells (iPSCs). Suitable cells also include myogenic cells differentiated from embryonic stem cells or iPSCs using methods known in the art including, but not limited to myogenic cells differented by methods and compositions disclosed in WO 2013/138623, the disclosure, methods, and compositions related to the differentiation of muscle cells or myogenic cells is incorporated by reference herein in its entirety. A “mixed” population of cells is a population of cells of varying degrees of developmental potency. For example, a mixed population of cells may comprise stem cells, progenitor cells, and/or differentiated cells in any suitable ratio.

In one embodiment, the starting population of cells is selected from adult or neonatal stem/progenitor cells. In particular embodiments, the starting population of stem/progenitor cells is selected from the group consisting of: mesodermal stem/progenitor cells, endodermal stem/progenitor cells, and ectodermal stem/progenitor cells.

Illustrative examples of mesodermal stem/progenitor cells include, but are not limited to: mesodermal stem/progenitor cells, endothelial stem/progenitor cells, bone marrow stem/progenitor cells, umbilical cord stem/progenitor cells, adipose tissue derived stem/progenitor cells, hematopoietic stem/progenitor cells (HSCs), mesenchymal stem/progenitor cells, muscle stem/progenitor cells, kidney stem/progenitor cells, osteoblast stem/progenitor cells, chondrocyte stem/progenitor cells, and the like.

Illustrative examples of ectodermal stem/progenitor cells include, but are not limited to neural stem/progenitor cells, retinal stem/progenitor cells, skin stem/progenitor cells, and the like.

Illustrative examples of endodermal stem/progenitor cells include, but are not limited to liver stem/progenitor cells, pancreatic stem/progenitor cells, epithelial stem/progenitor cells, and the like.

In preferred embodiments, the starting cell population comprises myogenic cells including, but not limited to mesoangioblasts, satellite cells, satellite stem cells, satellite progenitor cells, myoblasts, myocytes, Pax7⁺/Myf5⁻/MyoD⁻ cells, Pax7⁺/Myf5⁺/MyoD⁻ cells, and/or Pax7⁺/Myf5⁺/MyoD⁺ cells, or any suitable combination thereof

In other preferred embodiments, the starting cell population comprises Pax7⁺/Myf5⁻/MyoD⁻ cells, Pax7⁺/Myf5⁺/MyoD⁻ cells, and/or Pax7⁺/Myf5⁺/MyoD⁺ cells.

Cells contemplated for therapeutic use may be autologous/autogeneic (“self”) or non-autologous (“non-self,” e.g., allogeneic, syngeneic or xenogeneic) cells. “Autologous,” as used herein, refers to cells from the same subject. “Allogeneic,” as used herein, refers to cells of the same species that differ genetically to the cell in comparison. “Syngeneic,” as used herein, refers to cells of a different subject that are genetically identical to the cell in comparison. “Xenogeneic,” as used herein, refers to cells of a different species to the cell in comparison.

In preferred embodiments, the cells contemplated herein are allogeneic.

In particular embodiments, the cells contemplated herein are HLA typed and may be matched or partially matched to a specific patient for transplantation. HLA-type refers to the unique set of proteins called human leukocyte antigens. These proteins are present on each individual's cells and allow the immune system to recognize “self” from “foreign.” Administration of cells or tissues that are recognized as foreign can lead to compatibility problems such as immuno-rejection or graft versus host disease (GVHD). Accordingly, HLA type and matching is particularly important in organ and tissue transplantation.

There are six major HLAs (HLA-A, HLA-B, HLA-C, HLA-DR, HLA-DP, and HLA-DQ). Each HLA antigen has multiple isoforms in the human population, and each individual can have two different isoforms for each HLA due to the diploid nature of our genome. Therefore, a complete match would match twelve out of twelve isoforms. A cell or tissue donated from the same individual as, or an identical twin of, the intended recipient would have a perfect HLA-type and is referred to as syngeneic or autologous. It is also understood that certain factors including but not limited to ethnic background and race correlate with certain HLA-types.

Many major and minor HLA isoforms exist and it is understood that a suitable match may include a match between a subset of the major HLAs, all the major HLAs, some or all major and minor HLAs or any combination known to the art that mitigates immuno-rejection or GVDH. It is also understood that specific guidelines for what constitutes a good HLA-type match depends on many factors. Therefore, judgment must be made by one skilled in the art to assess the suitability of a given cell or tissue sample for transplant into a given individual.

HLA-type can be determined using so-called low resolution methods, for example by sero-typing, or using antibody based methods. Sero-typing is based on antibody recognition of HLA-types. Sero-typing can distinguish between 28 different HLA-A genes, 59 HLA-B genes and 21 HLA-C genes. A perfect match by sero-typing methods would be a so-called six out of six match referring to the two alleles for each HLA (A, B, and C) present in each individual. In certain cases, a five out of six match or less may be considered a good match as determined by one skilled in the art.

Other low or medium resolution methods to determine HLA-type examine the HLA isoforms of the individual, but do not rely on determining the actual sequence of an individual's HLA alleles. Often, the donor is related to the individual receiving the sample, in this case sero-typing alone or in combination with other low or medium resolution methods may be sufficient to determine if a sample is suitable for transplantation. In other cases a five out of six or lower match is readily found, but a perfect match is not. In such cases it may be advantageous to use cells or tissues with a lower match rather than expend time and effort to find a better HLA-type match.

High resolution methods involve examining the specific sequence of the HLA genes or gene expression products (protein or RNA). High resolution methods can distinguish between thousands of different isoforms. At a minimum, HLA typing of the therapeutic composition is performed for six HLA loci, HLA-A, -B, and -DR, for example, at low resolution/split antigen level.

DNA-based testing methods can be utilized for HLA-DR typing. DNA-based testing may be used for HLA-A and -B. Transplant center guidelines for typing of patient, family and to confirm the HLA types of potential unrelated donors include, typing HLA-A, B, and -DR loci using primarily DNA-based testing methods at allele level resolution for DRBl and low resolution/split antigen level for HLA-A and -B. The typing of a patient and the selected donor can be performed using the same set of reagents, methodology, and interpretation criteria with fresh tissue samples to ensure HLA identity. Quality assurance and quality control for HLA testing are complied with.

In various embodiments, the population of cells comprises haplotyped myogenic cells including, but not limited to, mesoangioblasts, satellite cells, satellite stem cells, satellite progenitor cells, myoblasts, myocytes, Pax7⁺/Myf5⁻/MyoD⁻ cells, Pax7⁺/Myf5⁺/MyoD⁻ cells, and Pax7⁺/Myf5⁺/MyoD⁺ cells, or mixtures thereof. In some embodiments, the population of cells is HLA typed based on HLA-A, HLA-B, HLA-C, and HLA-DRB1. In particular embodiments, the population of cells is HLA typed based on the group consisting of HLA-DRB3/4/5, HLA-DQB1, and DPB1. In some embodiments, the population of cells is matched with a specific human patient. In some embodiments, the population of HLA haplotyped cells has 4 out of 6 HLA matches with a specific human subject. HLA matching may be based on alleles or antigens, and combinations thereof. In some embodiments, the population of HLA haplotyped cells is a partial mismatch with a specific human subject, such as the subject to which the therapeutic cell-based composition is administered.

2. Therapeutic Cells

In various embodiments, one or more therapeutic cells or a population of cells comprising one or more therapeutic cells is provided. To generate therapeutic cells, non-canonical Wnt signaling is increased in the cells. Non-canonical Wnt signaling may be increased in the cells by contacting or culturing the cells in the presence of, or introducing into the cells, one or more non-canonical Wnt signaling activators as contemplated herein. In preferred embodiments, non-canonical Wnt signaling is increased in myogenic cells for a time sufficient to increase engraftment potential of the cells, and/or increase the ability of the transplanted cells to disperse from the administration site, to undergo myofusion, to increase force generation, to increase twitch tension, to increase cell motility, and/or increase cell migration.

In one embodiment, a culture of therapeutic cells and one or more non-canonical Wnt signaling activators is provided. In a particular embodiment, the culture comprises one or more myogenic cells including, but not limited to Pax7+/Myf5−/MyoD− stem cells, Pax7+/Myf5+/MyoD− progenitors cells, and Pax7+/Myf5+/MyoD+ myoblasts, and an exogenous non-canonical Wnt signaling pathway activator in an amount sufficient to increase one or more therapeutic properties of the cells, e.g., engraftment potential.

In one embodiment, a population of cells is contacted with, cultured with, or modified to express one or more non-canonical Wnt signaling pathway activators to increase one or more therapeutic properties of the cells. In another embodiment, one or more non-canonical Wnt signaling pathway activators may be introduced into the cells to increase one or more therapeutic properties of the cells. A cell may be cultured in the presence of a small molecule activator of non-canonical Wnt signaling; by contacting or introducing into the cells, one or more polypeptides that increases non-canonical Wnt signaling; or by contacting or introducing into the cells, one or more polynucleotides that increase non-canonical Wnt signaling in the cells.

In a particular embodiment, a myogenic cell is contacted with an effective amount of a small molecule activator of non-canonical Wnt signaling for a sufficient time to increase non-canonical Wnt signaling in the cell, and to increase one or more therapeutic properties in the cell, e.g., engraftment potential.

In a certain embodiment, a myogenic cell is contacted with an effective amount of a polypeptide that activates non-canonical Wnt signaling for a sufficient time to increase non-canonical Wnt signaling in the cell, and to increase one or more therapeutic properties in the cell, e.g., engraftment potential.

In one embodiment, a myogenic cell is contacted with an effective amount of a polynucleotide that encodes a polypeptide that activates non-canonical Wnt signaling for a sufficient time to increase non-canonical Wnt signaling in the cell, and to increase one or more therapeutic properties in the cell, e.g., engraftment potential. In particular embodiments, the polynucleotide modifies the genome of the cell. In other particular embodiments, the polynucleotide is episomal.

In one embodiment, a population of cells is treated (e.g., contacted) with one or more non-canonical Wnt signaling pathway activators, each at a final concentration of about 1 μM to about 100 μM. In certain embodiments, a population of cells is treated with one or more pharmaceutical agents, each at a final concentration of about 1×10⁻¹⁴ M to about 1×10⁻³ M, about 1×10⁻¹³ M to about 1×10⁻⁴ M, about 1×10⁻¹² M to about 1×10⁻⁵ M, about 1×10⁻¹¹ M to about 1×10⁻⁴ M, about 1×10⁻¹¹ M to about 1×10⁻⁵ M, about 1×10⁻¹⁰ M to about 1×10⁻⁴ M, about 1×10⁻¹⁰ M to about 1×10⁻⁵ M, about 1×10⁻⁹ M to about 1×10⁻⁴ M, about 1×10⁻⁹ M to about 1×10⁻⁵ M, about 1×10⁻⁸ M to about 1×10⁻⁴ M, about 1×10⁻⁷ M to about 1×10⁻⁴ M, about 1×10⁻⁶ M to about 1×10⁻⁴ M, or any intervening ranges of final concentrations.

In another particular embodiment, a population of cells is treated with one or more non-canonical Wnt signaling pathway activators, each at a final concentration of about 1×10⁻¹⁴ M, about 1×10⁻¹³ M, about 1×10⁻¹² M, about 1×10⁻¹⁰ M, about 1×10⁻⁹ M, about 1×10⁻⁸ M, about 1×10⁻⁷ M to about 1×10⁻⁶ M, about 1×10⁻⁵ M, about 1×10⁻⁴ M, about 1×10⁻³ M, or any intervening final concentration. In treatments comprising one or more one or more non-canonical Wnt signaling pathway activators, the activators can be at different concentrations from each other or at the same concentration.

In particular embodiments, a population of cells is treated (e.g., contacted with one or more non-canonical Wnt signaling pathway activators) 1, 2, 3, 4, 5, 6, 7, 8, 9 or 10 or more times. A population of cells can be intermittently, episodically, or sequentially contacted with one or more non-canonical Wnt signaling pathway activators.

In certain embodiments, a population of cells is cultured or treated with one or more non-canonical Wnt signaling pathway activators for about 1 hr, about 2 hours, about 4 hours, about 8 hours, about 16 hours, about 24 hours; about 2, 3, 4, 5, 6, or 7 days; about 1, 2, 3, or 4 weeks; about 1, 2, 3, 4, 5, 6 months, or longer, including any intervening duration of time, so long as the therapeutic properties of the cells are maintained. In addition, cells may be subject to repeated freeze/thaw cycles, prior to administration to a subject.

The therapeutic cells contemplated herein are capable of obtaining product licensure from the FDA (i.e., FDA approval) and other health authorities in other countries and regulatory territories, as well as product labeling with characterizing information regarding product indication, product efficacy, safety and purity. FDA licensure is likely to be based on cell dose and HLA mismatch. In particular embodiments, the therapeutic is processed and cryopreserved according to accredited standards, sterile, and labeled for, e.g., HLA typing and the A, B, and DR-beta-1 loci, and post-processing counts, infectious disease screening, family history and evidence of maternal consent for donation. The therapeutic cell composition to be used for transplant would include cells that match a minimum of 4/6 antigens or 3/6 alleles, and a cell dose as described herein.

3. Gene Therapy Compositions

In various embodiments, a starting population of cells is modified to provide gene therapy and then contacted with, cultured in the presence of, or modified to express one or more non-canonical Wnt signaling pathway activators. In other particular embodiments, the polynucleotide is episomal. In particular embodiments, the polynucleotide genetically modifies the cell. As used herein, the term “genetically modified” refers to the addition, deletion, or modification of the genetic material in a cell.

In other embodiments, a starting population of cells is contacted with, cultured in the presence of, or modified to express one or more non-canonical Wnt signaling pathway activators and provides gene therapy by virtue of providing a wild type or normal copy of the genome to an affected tissue of a subject.

In various embodiments, the genetically modified cells contemplated herein by introducing a polynucleotide encoding a therapeutic polypeptide or polypeptide-of-interest into the cell, in vitro or ex vivo, and optionally expanding the cells. The genetically modified cells are then administered to a subject in need of gene therapy. Without wishing to be bound to any particular theory, it is contemplated that genetically modified myogenic cells administered to the subject efficiently disperse, engraft, and fuse with host myogenic cells to form multinucleate syncytia, thereby delivering the polynucleotide encoding a therapeutic polypeptide or polypeptide-of-interest to the subject and providing efficient and long-lasting gene therapy.

Cells suitable for transduction and administration in the gene therapy methods contemplated herein include, but are not limited to a cell population comprises one or more stem cells, progenitor cells, and/or differentiated cells. In a preferred embodiment, a cell population comprising myogenic cells includes, satellite cells, satellite stem cells, satellite progenitor cells, myoblasts, myocytes, Pax7⁺/Myf5⁻/MyoD⁻ cells, Pax7⁺/Myf5⁺/MyoD⁻ cells, and/or Pax7⁺/Myf5⁺/MyoD⁺ cells, or combinations thereof are modified to provide gene therapy to a subject in need thereof.

In one embodiment, a polynucleotide that encodes a polypeptide that provides gene therapy is introduced into a myogenic cell. In certain embodiments, a heterogeneous population of genetically modified cells is contemplated, e.g., 5%, 10%, 15%, 20%, 25%, 30%, 35%, 40%, 45%, 50%, 55%, 60%, 65%, 70%, 75%, 80%, 85%, 90%, 95% or more cells are genetically modified. In other embodiments, a homogenous population of genetically modified cells is contemplated.

Without limitation, it is contemplated that the cells may be genetically modified with any polynucleotide that encodes a polypeptide that provides gene therapy contemplated herein. Generally, delivery of polynucleotides can be accomplished by, for example, dextran-mediated transfection, calcium phosphate precipitation, polybrene mediated transfection, protoplast fusion, electroporation, encapsulation of the polynucleotide(s) in liposomes, direct microinjection of the DNA into nuclei, and viral transduction, all of which are well known in the art.

In certain embodiments, it will be preferred to deliver a polynucleotide encoding a therapeutic polypeptide to the cell being genetically modified using a viral vector. In a preferred embodiment, such as vectors include adenovirus, retrovirus, lentivirus, adeno-associated virus vectors (AAV), or the use of other viral vectors as expression constructs (including without limitation vaccinia virus, polioviruses and herpes viruses).

The genetically modified cells contemplated herein, may be modified with a polynucleotide encoding a therapeutic polypeptide that provides regenerative therapy or gene therapy to a subject.

In one embodiment, cells are genetically modified with a polynucleotide encoding a therapeutic polypeptide that provides a therapeutic benefit to a subject having or diagnosed with a muscle wasting disease, such as cachexia, muscular attenuation or atrophy, including sarcopenia, ICU-induced weakness, surgery-induced weakness (e.g., following knee or hip replacement), muscle trauma, muscle injury, surgery, disuse atrophy, and muscle degenerative diseases, such as muscular dystrophies.

In one embodiment, cells are genetically modified with a polynucleotide encoding a therapeutic polypeptide that provides a therapeutic benefit to a subject having or diagnosed with a muscular dystrophy selected from the group consisting of Duchenne muscular dystrophy (DMD), Becker muscular dystrophy (BMD), Emery-Dreifuss muscular dystrophy, Landouzy-Dejerine muscular dystrophy, facioscapulohumeral muscular dystrophy (FSH), Limb-Girdle muscular dystrophies, von Graefe-Fuchs muscular dystrophy, oculopharyngeal muscular dystrophy (OPMD), Myotonic dystrophy (Steinert's disease) and congenital muscular dystrophies.

In one embodiment, cells are genetically modified with a polynucleotide encoding a laminin subunit, a subunits of the dystrophin glycoprotein complex (DGC), a dystrophin polypeptide or a Wnt polypeptide.

Illustrative examples of Wnt polypeptides that cells contemplated herein can be genetically modified to express include, but are not limited to human Wnt proteins selected from the group consisting of: Wnt1, Wnt2, Wnt2b/13, Wnt3, Wnt3a, Wnt4, Wnt5a, Wnt5b, Wnt6, Wnt7a, Wnt7b, Wnt8, Wnt8a, Wnt8b, Wnt9a, Wnt9b, Wnt10a, Wnt10b, Wnt11, Wnt16, orthologs, paralogs, homologs, and modified Wnt polypeptides thereof.

### E. Methods for Administering Cell Based Compositions

The therapeutic compositions contemplated herein are sterile, and are suitable and ready for administration (i.e., can be administered without any further processing) to human patients. As used herein, the terms “administration-ready” or “ready for administration” refer to a cell-based composition contemplated herein that does not require any further treatment or manipulations prior to transplant or administration to a subject.

The sterile, therapeutically acceptable compositions suitable for administration to a patient may comprise one or more pharmaceutically-acceptable salts, carriers, diluents, excipients, and/or physiologically-acceptable solutions (e.g., pharmaceutically acceptable medium, for example, cell culture medium), or other pharmaceutically acceptable components. Pharmaceutically acceptable carriers and/or diluents are determined in part by the particular composition being administered, as well as by the particular method used to administer the therapeutic composition.

A “pharmaceutical composition” refers to a formulation of a composition of the invention and a medium generally accepted in the art for the delivery of cell-based therapeutics to mammals, e.g., humans. Such a medium includes all pharmaceutically acceptable salts, carriers, diluents or excipients. Additional methods of formulating compositions known to the skilled artisan, for example, as described in the Physicians Desk Reference, 62nd edition. Oradell, N.J.: Medical Economics Co., 2008; Goodman & Gilman's The Pharmacological Basis of Therapeutics, Eleventh Edition. McGraw-Hill, 2005; Remington: The Science and Practice of Pharmacy, 20th Edition. Baltimore, Md.: Lippincott Williams & Wilkins, 2000; and The Merck Index, Fourteenth Edition. Whitehouse Station, N.J.: Merck Research Laboratories, 2006; each of which is hereby incorporated by reference in relevant parts.

In other illustrative embodiments, a therapeutic composition or cell-based composition contemplated herein may comprise a biocompatible scaffold, optionally comprising a bioabsorbable material. A porous carrier is preferably made of one component or a combination of multiple components selected from the group consisting of collagen, collagen derivatives, hyaluronic acid, hyaluronates, chitosan, chitosan derivatives, polyrotaxane, polyrotaxane derivatives, chitin, chitin derivatives, gelatin, fibronectin, heparin, laminin, and calcium alginate; wherein a support member is made of one component or a combination of multiple components selected from the group consisting of polylactic acid, polyglycolic acid, polycaprolactone, polylactic acid-polyglycolic acid copolymer, polylactic acid-polycaprolactone copolymer, and polyglycolic acid-polycaprolactone copolymer (see, for example, U.S. Pat. Nos. 5,077,049 and 5,42,033, and U.S. Patent Application Publication No. 2006/0121085, of which the polymer formulations and methods of making the same of each patent and application is incorporated herein in its entirety).

In particular illustrative embodiments of the invention, the biocompatible scaffold or cell graft comprises a viscous, biocompatible liquid material. The biocompatible liquid is capable of gelling at body temperature and is selected from the group consisting of alginate, collagen, fibrin, hyaline, or plasma. The viscous, biocompatible liquid material can also be combined with a malleable, three dimensional matrix capable of filling an irregular tissue defect. The matrix is a material including, but not limited to, polyglycolic-polylactic acid, poly-glycolic acid, poly-lactic acid, or suture-like material.

In further illustrative embodiments, biocompatible scaffolds or cell grafts comprising matrices can be molded into desired shapes (e.g., two-dimensional or three-dimensional structures) conducive to or facilitating cell, tissue, and/or organ development. The implant can be formed from polymeric material, having fibers such as a mesh or sponge. Such a structure provides sufficient area on which the cells can grow and proliferate. Desirably, the matrices of the scaffolds or cell grafts are biodegradable over time, so that they will be absorbed into the animal matter as it develops. Suitable polymers can be homopolymers or heteropolymers and can be formed from monomers including, but not limited to glycolic acid, lactic acid, propyl fumarate, caprolactone, and the like. Other suitable polymeric material can include a protein, polysaccharide, polyhydroxy acid, polyorthoester, polyanhydride, polyphosphozene, or a synthetic polymer, particularly a biodegradable polymer, or any combination thereof.

In particular embodiments, therapeutic cell compositions comprising myogenic cells including, but not limited to satellite cells, satellite stem cells, satellite progenitor cells, myoblasts, myocytes, Pax7⁺/Myf5⁻/MyoD⁻ cells, Pax7⁺/Myf5⁺/MyoD⁻ cells, and/or Pax7⁺/Myf5⁺/MyoD⁺ cells are formulated in a pharmaceutically acceptable cell culture medium. A therapeutic composition comprising a cell-based composition contemplated herein can be administered parenterally. As used herein, the phrases “parenteral administration” and “administered parenterally” refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion. See, for example, in U.S. Pat. No. 5,543,158; U.S. Pat. No. 5,641,515 and U.S. Pat. No. 5,399,363 (each specifically incorporated herein by reference in its entirety).

The pharmaceutically-acceptable salts, carriers, diluents, excipients, and/or physiologically-acceptable solutions must be of sufficiently high purity and of sufficiently low toxicity to render it suitable for administration to the human subject being treated. It further should maintain or increase the stability of the therapeutic composition. The pharmaceutically acceptable carrier can be liquid or solid and is selected, with the planned manner of administration in mind, to provide for the desired bulk, consistency, etc., when combined with other components of the therapeutic composition of the invention. For example, the pharmaceutically acceptable carrier can be, without limitation, a binding agent (e.g., pregelatinized maize starch, polyvinylpyrrolidone or hydroxypropyl methylcellulose, etc.), a filler (e.g., lactose and other sugars, microcrystalline cellulose, pectin, gelatin, calcium sulfate, ethyl cellulose, polyacrylates, calcium hydrogen phosphate, etc.), a lubricant (e.g., magnesium stearate, talc, silica, colloidal silicon dioxide, stearic acid, metallic stearates, hydrogenated vegetable oils, corn starch, polyethylene glycols, sodium benzoate, sodium acetate, etc.), a disintegrant (e.g., starch, sodium starch glycolate, etc.), or a wetting agent (e.g., sodium lauryl sulfate, etc.). Other suitable pharmaceutically acceptable carriers for the compositions of the present invention include, but are not limited to, water, salt solutions, alcohols, polyethylene glycols, gelatins, amyloses, magnesium stearates, talcs, silicic acids, viscous paraffins, hydroxymethylcelluloses, polyvinylpyrrolidones and the like.

Such carrier solutions also can contain buffers, diluents and other suitable additives. The term “buffer” as used herein refers to a solution or liquid whose chemical makeup neutralizes acids or bases without a significant change in pH. Examples of buffers envisioned by the invention include, but are not limited to, Dulbecco's phosphate buffered saline (PBS), Ringer's solution, 5% dextrose in water (D5W), normal/physiologic saline (0.9% NaCl).

These pharmaceutically acceptable carriers and/or diluents may be present in amounts sufficient to maintain a pH of the therapeutic composition of between about 3 and about 10. As such, the buffering agent may be as much as about 5% on a weight to weight basis of the total composition. Electrolytes such as, but not limited to, sodium chloride and potassium chloride may also be included in the therapeutic composition.

In one aspect, the pH of the therapeutic composition is in the range from about 4 to about 10. Alternatively, the pH of the therapeutic composition is in the range from about 5 to about 9, from about 6 to about 9, or from about 6.5 to about 8. In another embodiment, the therapeutic composition comprises a buffer having a pH in one of said pH ranges. In another embodiment, the therapeutic composition has a pH of about 7. Alternatively, the therapeutic composition has a pH in a range from about 6.8 to about 7.4. In still another embodiment, the therapeutic composition has a pH of about 7.4.

The sterile composition of the invention may be a sterile solution or suspension in a nontoxic pharmaceutically acceptable medium. The term “suspension” as used herein may refer to non-adherent conditions in which cells are not attached to a solid support. For example, cells maintained in suspension may be stirred and are not adhered to a support, such as a culture dish.

A suspension is a dispersion (mixture) in which a finely-divided species is combined with another species, with the former being so finely divided and mixed that it doesn't rapidly settle out. A suspension may be prepared using a vehicle such as a liquid medium, including a solution. In particular embodiments, the therapeutic composition of the invention is a suspension, where the myogenic cells are dispersed within an acceptable liquid medium or solution, e.g., saline or serum-free medium, and are not attached to a solid support. In everyday life, the most common suspensions are those of solids in liquid water. Among the acceptable diluents, e.g., vehicles and solvents, that may be employed are water, Ringer's solution, isotonic sodium chloride (saline) solution, and serum-free cell culture medium. In some embodiments, hypertonic solutions are employed in making suspensions. In addition, sterile, fixed oils are conventionally employed as a solvent or suspending medium. For parenteral application, particularly suitable vehicles consist of solutions, preferably oily or aqueous solutions, as well as suspensions, emulsions, or implants. Aqueous suspensions may contain substances which increase the viscosity of the suspension and include, for example, sodium carboxymethyl cellulose, sorbitol and/or dextran. In some embodiments, the infusion solution is isotonic to subject tissues. In some embodiments, the infusion solution is hypertonic to subject tissues.

The pharmaceutically-acceptable salts, carriers, diluents, excipients, and/or physiologically-acceptable solutions, and other components comprising the administration-ready therapeutic composition of the invention are derived from U.S. Pharmaceutical grade reagents that will permit the therapeutic composition to be used in clinical regimens. Typically, these finished reagents, including any medium, solution, or other pharmaceutically acceptable carriers and/or diluents, are sterilized in a manner conventional in the art, such as filter sterilized, and are tested for various undesired contaminants, such as mycoplasma, endotoxin, or virus contamination, prior to use. The pharmaceutically acceptable carrier in one embodiment is substantially free of natural proteins of human or animal origin, and suitable for storing the population of cells of the therapeutic composition, including myogenic cells. The therapeutic composition is intended to be administered into a human patient, and thus is substantially free of cell culture components such as bovine serum albumin, horse serum, and fetal bovine serum.

The use of a pharmaceutically acceptable cell culture medium in particular compositions and/or cultures is contemplated in certain embodiments. Such compositions are suitable for administration to human subjects. Generally speaking, any medium that supports the maintenance, growth, and/or health of the desired reprogrammed and/or programmed cells of the invention are suitable for use as a pharmaceutical cell culture medium. In particular embodiments, the pharmaceutically acceptable cell culture medium is a serum free medium.

The therapeutic composition may comprise serum-free medium suitable for storing the population of cells comprising the composition. Serum-free medium has several advantages over serum containing medium, including a simplified and better defined composition, a reduced degree of contaminants, elimination of a potential source of infectious agents, and lower cost. In various embodiments, the serum-free medium is animal-free, and may optionally be protein-free. Optionally, the medium may contain biopharmaceutically acceptable recombinant proteins. “Animal-free” medium refers to medium wherein the components are derived from non-animal sources. Recombinant proteins replace native animal proteins in animal-free medium and the nutrients are obtained from synthetic, plant or microbial sources. Protein-free medium, in contrast, is defined as substantially free of protein.

The serum-free medium employed in the present invention is a formulation suitable for use in human therapeutic protocols and products. Serum-free media known in the art include, but are not limited to: Life Technologies: Knock Out DMEM+XF KSR; Biological Industries: NutriStem; Life Technologies Catalogue StemPro-34 serum free culture media; Life Technologies Catalogue information on AIM V serum free culture media; BioWhittaker Catalogue information on X-VIVO 10 serum free culture media; U.S. Pat. No. 5,397,706 entitled Serum-free basal and culture medium for hematopoietic and leukemia cells; no cell proliferation; Kurtzberg et al., 18:153-4 (2000); Kurtzberg et al., Exp Hematol 26(4):288-98 (April 1998).

One having ordinary skill in the art would appreciate that the above example of medium is illustrative and in no way limits the formulation of media suitable for use in the present invention and that there are many such media known and available to those in the art.

The therapeutic composition is substantially free of mycoplasm, endotoxin, and microbial contamination. In particular embodiments, the therapeutic composition contains less than about 10, 5, 4, 3, 2, 1, 0.1, 0.05 μg/ml bovine serum albumin.

By “substantially free” with respect to endotoxin is meant that there is less endotoxin per dose of cells than is allowed by the FDA for a biologic, which is a total endotoxin of 5 EU/kg body weight per day, which for an average 70 kg person is 350 EU per total dose of cells.

With respect to mycoplasma and microbial contamination, “substantially free” as used herein means a negative reading for the generally accepted tests known to those skilled in the art. For example, mycoplasm contamination is determined by subculturing a sample of the therapeutic composition in broth medium and distributed over agar plates on day 1, 3, 7, and 14 at 37° C. with appropriate positive and negative controls. The sample appearance is compared microscopically, at 100×, to that of the positive and negative control. Additionally, inoculation of an indicator cell culture is incubated for 3 and 5 days and examined at 600× for the presence of mycoplasmas by epifluorescence microscopy using a DNA-binding fluorochrome. The sample is considered satisfactory if the agar and/or the broth media procedure and the indicator cell culture procedure show no evidence of mycoplasma contamination.

### F. Methods of Treatment

The therapeutic cell compositions contemplated herein including, but not limited to, cells comprising increased non-canonical Wnt signaling, and optionally genetically modified with a polynucleotide encoding a therapeutic polypeptide are useful for various therapeutic applications. In particular embodiments, the compositions and methods contemplated herein are useful for promoting tissue formation, regeneration, repair or maintenance in a subject in need thereof. In other particular embodiments, the compositions and methods contemplated herein are useful for increasing force generation, to increasing twitch tension; and/or hypertrophy in a subject in need thereof.

In preferred embodiments, the compositions and methods contemplated herein are useful in enhancing the engraftment of a cell therapy, increasing the efficacy of a cell graft in a subject, delivering a polynucleotide encoding a therapeutic polypeptide or polypeptide-of-interest to a subject, providing a myogenic cell-based gene therapy to a subject, and treating, ameliorating, or preventing a muscular disorder in a subject.

In various embodiments, a method of increasing engraftment of a cell in a subject comprises contacting the cell or culturing the cell in vitro or ex vivo with or introducing into the cell at least one non-canonical Wnt signaling activator for a time sufficient to increase non-canonical Wnt signaling in the cell and administering the cell to a subject in need thereof. The administered cells having increased non-canonical Wnt signaling have increased engraftment as a function of increased dispersal from the administration site and increased cell motility and/or migration of the cell compared to a non-contacted cell.

In particular embodiments, the engraftment of myogenic cells is increased by contacting or culturing myogenic cells or introducing into the myogenic cells one or more non-canonical Wnt signaling activators. In one embodiment, the one or more non-canonical Wnt signaling activators increase non-canonical Wnt7a/Fzd7 signaling in the myogenic cells. In addition to increased engraftment, upon administration to a subject, the contacted myogenic cells may also display increases in one or more of the following therapeutic properties: dispersal, cell motility, cell migration, myofusion, twitch tension, force generation, and hypertrophy.

In one embodiment, a method of increasing cell graft, i.e., a population of cells to be transplanted in a subject, efficacy in a subject comprises contacting a cell graft in vitro with a non-canonical Wnt signaling activator for a time sufficient to increase the engraftment potential of the cell graft and administering the cell graft to a subject in need thereof, wherein the administered cell graft has increased engraftment compared to a non-contacted cell graft.

In preferred embodiments, a population of cells or cell graft comprising mesoangioblast cells, satellite cells, satellite stem cells, satellite progenitor cells, myoblasts, myocytes, Pax7⁺/Myf5⁻/MyoD⁻ cells, Pax7⁺/Myf5⁺/MyoD⁻ cells, and/or Pax7⁺/Myf5⁺/MyoD⁺ cells is contacted with or modified to express at least one non-canonical Wnt signaling activator that increases non-canonical Wnt7a/Fzd7 signaling. In one preferred embodiment, the cells are genetically modified with a polynucleotide encoding a therapeutic polypeptide and are suitable for providing gene therapy to a subject in need thereof.

In various embodiments, a myogenic cell-based gene therapy is contemplated. In particular embodiments, a myogenic cell-based gene therapy comprises a myogenic cell genetically modified or altered with a polynucleotide encoding a therapeutic polypeptide or polypeptide-of-interest, contacting the genetically modified myogenic cell with or introducing into the cell, a non-canonical Wnt signaling activator for a time sufficient to increase non-canonical Wnt signaling in the cell and administering the contacted myogenic cell to a subject in need of gene therapy. In particular embodiments, the polynucleotide encoding a therapeutic polypeptide or polypeptide-of-interest is delivered to the subject when the myogenic cell fuses and forms a multinucleate syncytium with the affected muscle cells in the subject.

In preferred embodiments, myogenic cells comprising mesoangioblast cells, satellite cells, satellite stem cells, satellite progenitor cells, myoblasts, myocytes, Pax7⁺/Myf5⁻/MyoD⁻ cells, Pax7⁺/Myf5⁺/MyoD⁻ cells, and/or Pax7⁺/Myf5⁺/MyoD⁺ cells are contacted with or modified to express at least one non-canonical Wnt signaling activator that increases non-canonical Wnt7a/Fzd7 signaling.

In another preferred embodiment, the cells are genetically modified with a polynucleotide encoding a therapeutic polypeptide that provides a therapeutic benefit to a subject having or diagnosed with a muscle wasting disease, such as cachexia, muscular attenuation or atrophy, including sarcopenia, ICU-induced weakness, surgery-induced weakness (e.g., following knee or hip replacement), muscle trauma, muscle injury, surgery, disuse atrophy, and muscle degenerative diseases, such as muscular dystrophies.

Illustrative examples of muscular dystrophies that can be treated with myogenic cell-based gene therapies contemplated herein include: Duchenne muscular dystrophy (DMD), Becker muscular dystrophy (BMD), Emery-Dreifuss muscular dystrophy, Landouzy-Dejerine muscular dystrophy, facioscapulohumeral muscular dystrophy (FSH), Limb-Girdle muscular dystrophies, von Graefe-Fuchs muscular dystrophy, oculopharyngeal muscular dystrophy (OPMD), Myotonic dystrophy (Steinert's disease) and congenital muscular dystrophies.

In one preferred embodiment, myogenic cells are genetically modified with a polynucleotide encoding a laminin subunit, a subunits of the dystrophin glycoprotein complex (DGC), a dystrophin polypeptide, or a Wnt polypeptide including, but not limited to Wnt1, Wnt2, Wnt2b/13, Wnt3, Wnt3a, Wnt4, Wnt5a, Wnt5b, Wnt6, Wnt7a, Wnt7b, Wnt8, Wnt8a, Wnt8b, Wnt9a, Wnt9b, Wnt10a, Wnt10b, Wnt11, Wnt16, orthologs, paralogs, homologs, and modified Wnt polypeptide thereof.

In various embodiments, methods of delivering a polynucleotide encoding a therapeutic polypeptide or a polypeptide-of-interest to a subject in need thereof are contemplated. In particular embodiments, a myogenic cell is genetically modified or altered with a polynucleotide encoding a therapeutic polypeptide or polypeptide-of-interest, and the genetically modified myogenic cell is contacted with or modified to express a non-canonical Wnt signaling activator for a time sufficient to increase non-canonical Wnt signaling in the cell and the contacted myogenic cell is administered to a subject in need thereof. In particular embodiments, the polynucleotide encoding a therapeutic polypeptide or polypeptide-of-interest is delivered to the subject when the myogenic cell fuses and forms a multinucleate syncytium with the affected muscle cells in the subject.

In preferred embodiments, a polynucleotide is delivered to a subject via genetic modification of myogenic cells comprising satellite cells, satellite stem cells, satellite progenitor cells, myoblasts, myocytes, Pax7⁺/Myf5⁻/MyoD⁻ cells, Pax7⁺/Myf5⁺/MyoD⁻ cells, and/or Pax7⁺/Myf5⁺/MyoD⁺ cells that are contacted with or modified to express at least one non-canonical Wnt signaling activator that increases non-canonical Wnt7a/Fzd7 signaling.

Illustrative examples of therapeutic polypeptides suitable for delivering to a subject using the polynucleotide delivery systems contemplated herein include, but are not limited to: a laminin subunit, a subunits of the dystrophin glycoprotein complex (DGC), dystrophin, Wnt1, Wnt2, Wnt2b/13, Wnt3, Wnt3a, Wnt4, Wnt5a, Wnt5b, Wnt6, Wnt7a, Wnt7b, Wnt8, Wnt8a, Wnt8b, Wnt9a, Wnt9b, Wnt10a, Wnt10b, Wnt11, Wnt16, orthologs, paralogs, homologs, and modified polypeptides thereof

In various other embodiments, a method of preventing, ameliorating, or treating a muscle disorder or a symptom thereof in a subject in need thereof is provided. Symptoms of muscular disorders include, but are not limited to muscle wasting, decreased muscle mass, decreased twitch tension, decrease force generation, and muscle protein catabolism. In particular embodiments, a method of preventing, ameliorating, or treating a muscle disorder or a symptom thereof comprises contacting a myogenic cell with one or more non-canonical Wnt signaling pathway activators or introducing one or more non-canonical Wnt signaling pathway activators into the myogenic cell in vitro, optionally wherein the myogenic cell has been genetically altered a polynucleotide encoding a therapeutic polypeptide or polypeptide-of-interest, and administering the genetically altered myogenic cell to the subject.

In preferred embodiments, the myogenic cells comprise satellite cells, satellite stem cells, satellite progenitor cells, myoblasts, myocytes, Pax7⁺/Myf5⁻/MyoD⁻ cells, Pax7⁺/Myf5⁺/MyoD⁻ cells, and/or Pax7⁺/Myf5⁺/MyoD⁺ cells and are contacted with or modified to express at least one non-canonical Wnt signaling activator that increases non-canonical Wnt7a/Fzd7 signaling.

Some relevant indications that can be prevented, ameliorated, or treated with the compositions contemplated herein include situations where there is a need to prevent muscle loss or regenerate lost or damaged muscle tissue by increasing muscle size, volume or strength. Such situations may include, for example, after chemotherapy or radiation therapy, after muscle injury, or in the treatment or management of diseases and conditions affecting muscle. In certain embodiments, the disease or condition affecting muscle may include urinary incontinence, a wasting disease (e.g., cachexia, which may be associated with an illness such as cancer or AIDS), muscular attenuation or atrophy, or a muscle degenerative disease. Muscular attenuation and atrophy may be associated with, for example, sarcopenia (including age-related sarcopenia), ICU-induced weakness, disuse of muscle (for example disuse of muscle due to coma paralysis, injury, or immobilization), surgery-induced weakness (e.g., following hip or knee replacement), muscle trauma, muscle injury, surgery, disuse atrophy, or a muscle degenerative disease (e.g., muscular dystrophies). This list is not exhaustive.

In certain embodiments, the compositions contemplated herein may be used to replace or repair damaged or defective tissue, or to prevent muscle atrophy or loss of muscle mass, in particular, in relation to diseases and disorders affecting muscle, such as muscular dystrophy, neuromuscular and neurodegenerative diseases, muscle wasting diseases and conditions, atrophy, cardiovascular disease, stroke, heart failure, myocardial infarction, cancer, HIV infection, AIDS, and the like.

In additional embodiments, the compositions and methods contemplated herein are useful for repairing or regenerating dysfunctional skeletal muscle, for instance, in subjects having muscle degenerative diseases. The subject can be suspected of having, or be at risk of at having skeletal muscle damage, degeneration or atrophy. The skeletal muscle damage may be disease related or non-disease related. The human subject may have or be at risk of having muscle degeneration or muscle wasting. The muscle degeneration or muscle wasting may be caused in whole or in part by a disease, for example AIDS, cancer, a muscular degenerative disease, e.g., muscular dystrophy, or a combination thereof.

Illustrative examples of muscular dystrophies include, but are not limited to Duchenne muscular dystrophy (DMD), Becker muscular dystrophy (BMD), Emery-Dreifuss muscular dystrophy, Landouzy-Dejerine muscular dystrophy, facioscapulohumeral muscular dystrophy (FSH), Limb-Girdle muscular dystrophies, von Graefe-Fuchs muscular dystrophy, oculopharyngeal muscular dystrophy (OPMD), Myotonic dystrophy (Steinert's disease) and congenital muscular dystrophies.

All publications, patent applications, and issued patents cited in this specification are herein incorporated by reference as if each individual publication, patent application, or issued patent were specifically and individually indicated to be incorporated by reference in its entirety.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### EXAMPLES

### Example 1

### Wnt7a and Fzd7 Polarize Myogenic Cells and Stimulate Directed Cell Migration

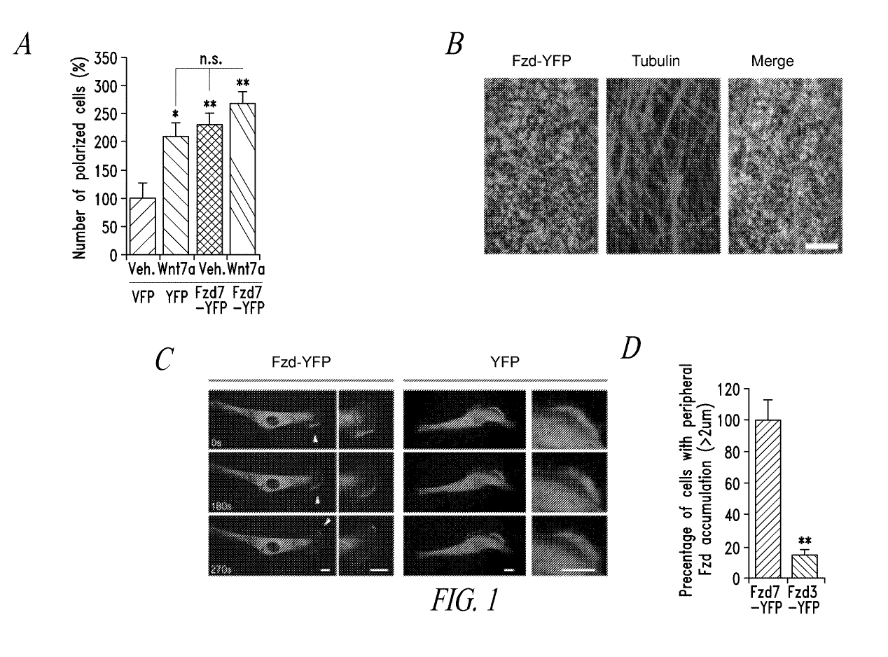

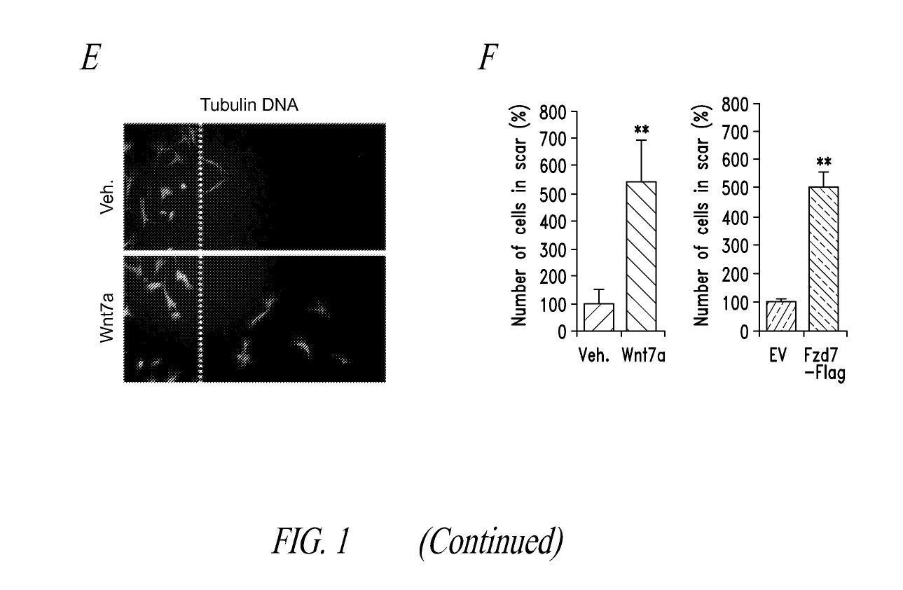

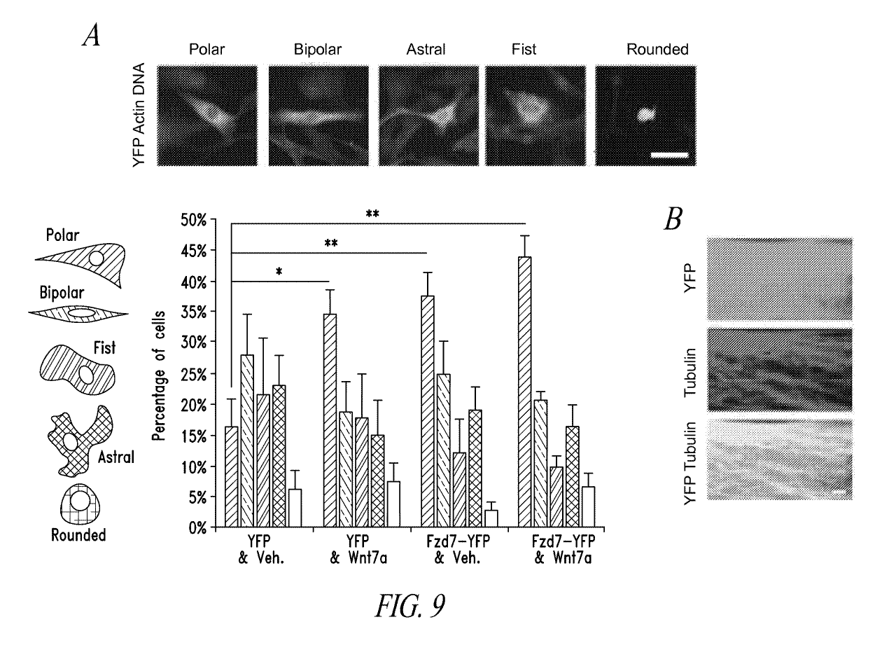

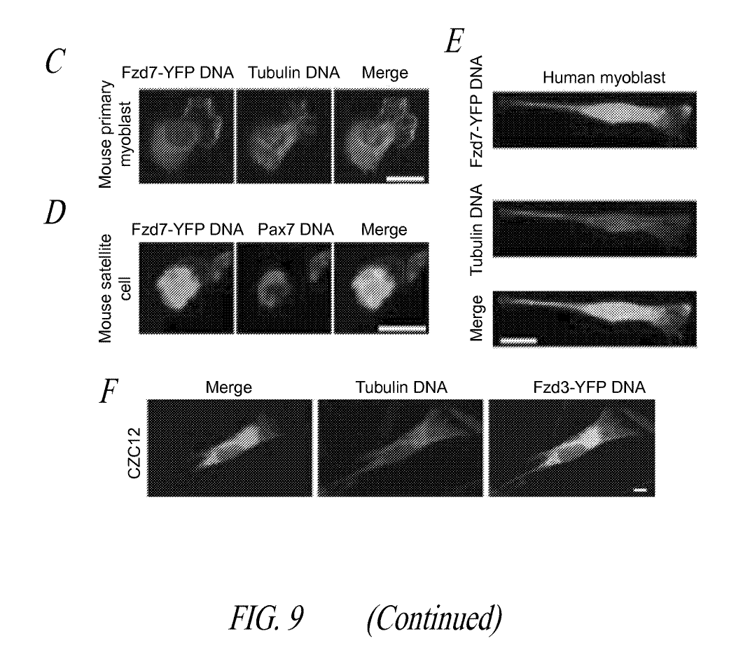

Cell migration is typically accompanied by cytoskeletal polarization leading to a distinctive triangular cell shape. C2C12 myoblasts incubated with Wnt7a led to a 111% increase in the abundance of triangular polarized cells (FIG. 1A and FIG. 9A). The major receptor of Wnt7a in myogenic cells is Fzd7 and Fzd7 overexpression has been shown to be sufficient to induce signaling.

Fzd7-YFP was expressed in myogenic cells and induced polarization by 130% (FIG. 1A and FIG. 9A). Treatment of Fzd7-YFP expressing cells with Wnt7a increased cell polarization by 167% but was not substantially different from the Wnt7a or Fzd7-YFP conditions alone. The majority of Fzd7-YFP localized to small intracellular vesicles associated with the tubulin cytoskeleton in C2C12 cells (FIG. 1B). By contrast, no such localization could be observed for YFP alone (FIG. 9B). It was observed that Fzd7-YFP also accumulated in the periphery of migrating cells (FIG. 1C), while YFP alone did not show such a polarized localization (FIG. 1C). In addition, peripheral localization of Fzd7-YFP was observed in transfected primary mouse myoblasts (FIG. 9C), mouse satellite cells (FIG. 9D) and human myoblasts (FIG. 9E). The peripheral localization of Fzd7-YFP was not due to its transmembrane nature because an unrelated canonical Fzd, Fzd3-YFP, did not exhibit this localization (FIG. 1D and FIG. 9F).

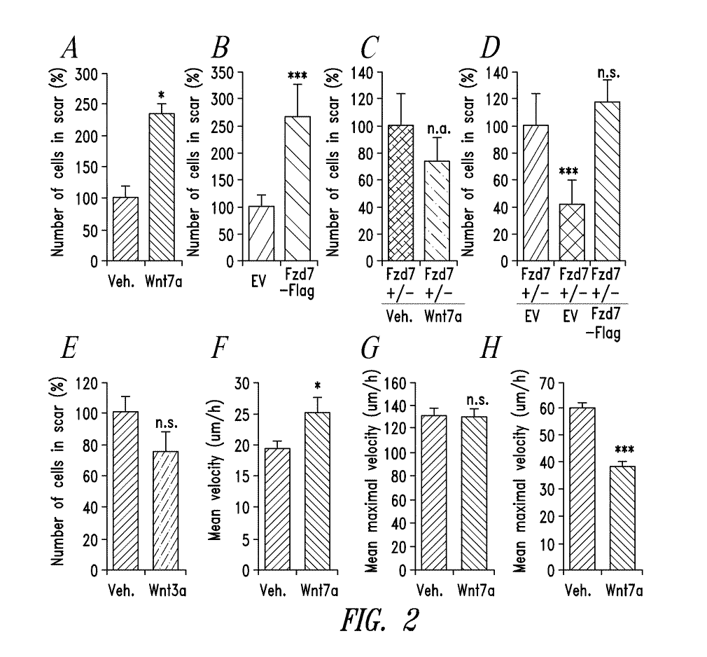

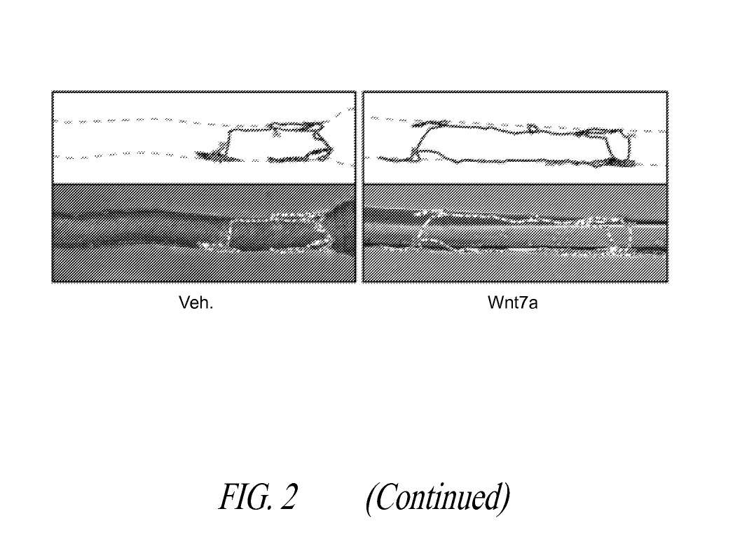

Cellular polarization is indicative of increased motility (de Forges et al., 2012). Scratch assays were used to test whether Wnt7a and Fzd7 polarization affect cell motility. To exclude effects on the localization of cells arising from altered rates of proliferation, cells were treated with Mitomycin-C. Addition of Wnt7a to the culture medium increased the migration of C2C12 cells by 443% (FIGS. 1E and 1F). Similarly, over-expression of Fzd7-Flag resulted in a 401% increase in cell migration (FIG. 1G). Wnt7a treatment and Fzd7-Flag overexpression also increased cell migration by 136% and 167% respectively in satellite cell derived mouse primary myoblasts (FIGS. 2A and 2B).

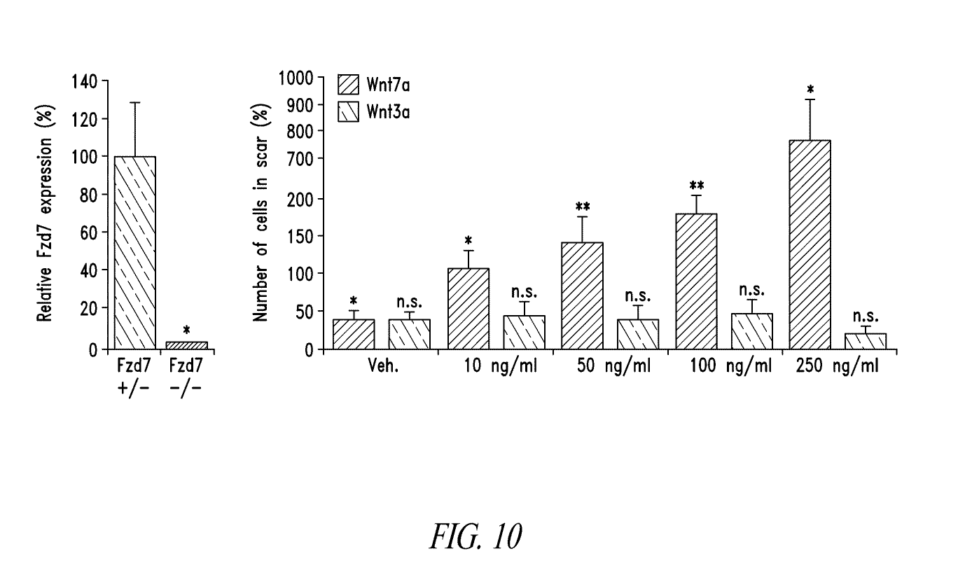

The effect of Wnt7a signaling on cell migration was dependent on Fzd7 since myoblasts derived from Fzd7 knockout mice showed no reaction to this factor (FIG. 2C and FIG. 10A). Fzd7 deficient cells migrated 59% less compared to heterozygous myoblasts and expression of Fzd7-Flag in Fzd7 knockout myoblasts restored migration to normal levels (FIG. 2D). An unrelated canonical Wnt, Wnt3a, did not stimulate cell migration of primary mouse myoblasts (FIG. 2E). To investigate whether the stimulation of myoblast migration is dose dependent, different concentrations of Wnt7a and Wnt3a were tested for their effect on migration. Increased concentrations of the non-canonical Wnt, Wnt7a, caused relatively linear increases in cell migration. In contrast, the canonical Wnt, Wnt3a, had no significant effect at any concentration tested (FIG. 10B).

The effect of Wnt7a on satellite cell migration was measured using a time lapse imaging technique previously employed to monitor satellite cells on single muscle myofibers (Siegel et al., 2009). Wnt7a increased the mean velocity of satellite cells by 31% when compared to Veh. (FIG. 2F). The mean maximal velocity was not substantially different between Wnt7a and Veh. (FIG. 2G). The increase in mean velocity was therefore likely caused by the 36% higher directional persistence of Wnt7a treated satellite cells (FIGS. 2H and 2I).

These data indicated that the activation of Wnt7a/Fzd7 signaling markedly stimulated the motility of satellite cells and myogenic progenitors by inducing polarization and enhancing directionality of migration.

### Example 2

### Wnt7a Induces Cell Migration

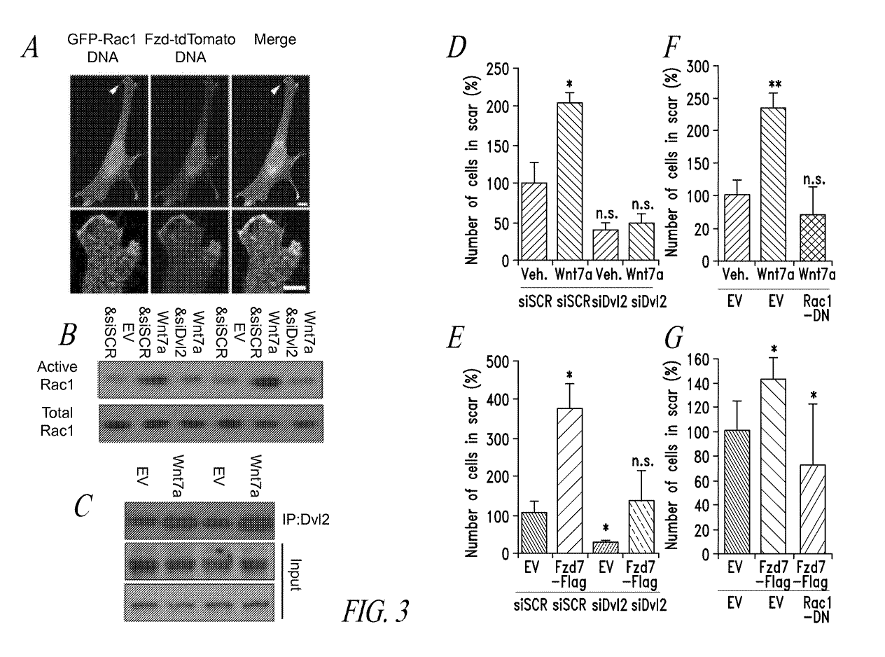

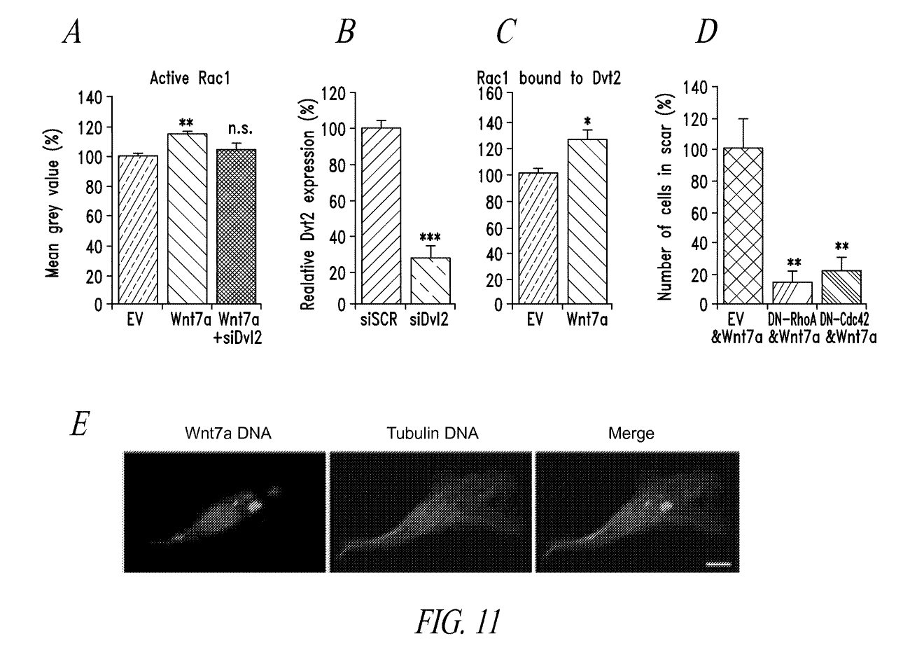

Wnt7a signaling in satellite cells involves Rac1. Polarized peripheral Fzd7-tdTomato colocalized with GFP-Rac1 in C2C12 cells (FIG. 3A). It was hypothesized that the small GTPase Rac1 might also be involved in Wnt7a mediated cell polarization and migration. In agreement with this hypothesis, Wnt7a overexpression significantly increased the activation of Rac1 (FIG. 3B and FIG. 11A). Various forms of Wnt signaling require Disheveled (Dvl) proteins. At the protein level, Dvl2 is the most expressed Dvl in mouse primary myoblasts. siRNA SMARTpool mediated knockdown of Dvl2 (siDvl2) from Wnt7a stimulated cells significantly reduced the activation of Rac1 when compared to the scrambled siRNA (siSCR) control (FIG. 3B; and FIGS. 11A and 11B). In addition, increased Rac1 association with Dvl2 was observed in Wnt7a stimulated cells (FIG. 3C and FIG. 11C).

siDvl2 prevented both Wnt7a and Fzd7-Flag overexpression mediated migration of mouse primary myoblasts in scratch assays (FIGS. 3D and 3E). Moreover, the expression of dominant negative Rac1-T17N (Rac1-DN) also antagonized Wnt7a and Fzd7 mediated migration in primary mouse myoblasts (FIGS. 3F and 3G). Biochemical analysis revealed no significant activation of other small GTPases such as Cdc42 and RhoA but both dominant negative Cdc42-T17N (Cdc42-DN) and RhoA-T19N (RhoA-DN) were able to prevent Wnt7a induced cell migration (FIG. 11D). Thus, these data indicated that Dvl2 and the small GTPases Rac1, Cdc42 and RhoA play a role in the induction of cell motility by Wnt7a.

### Example 3

### Non-Canonical Wnt7a Signaling in Myoblasts

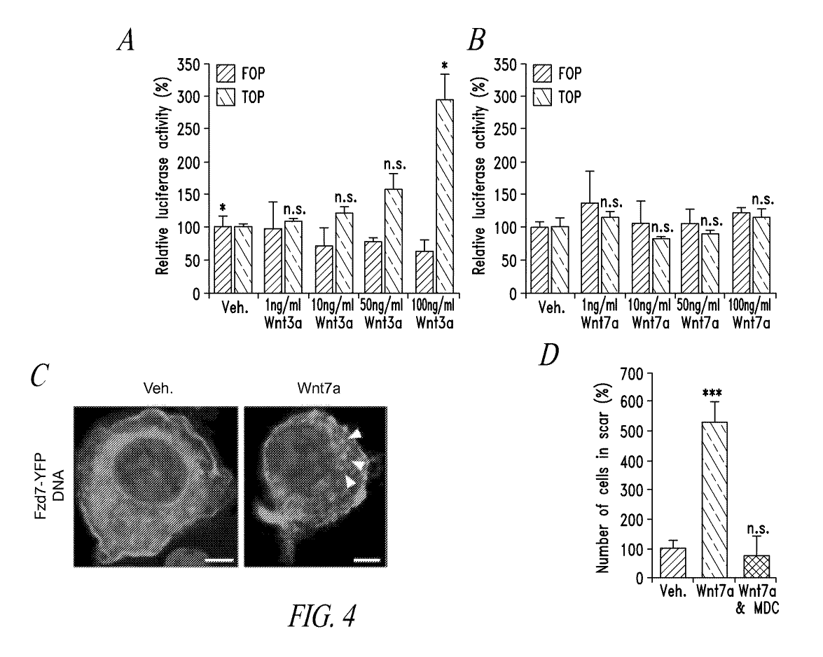

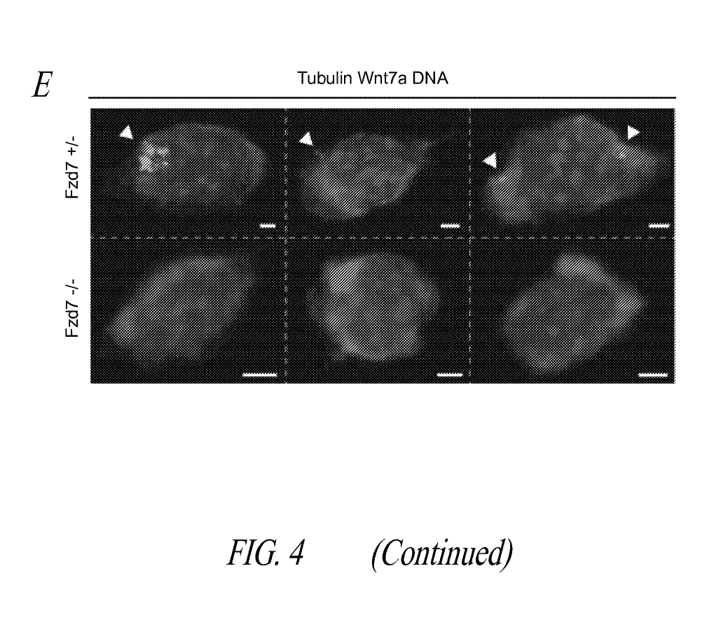

Wnt7a does not appear to activate β-catenin signaling in satellite cells, primary myoblasts (Le Grand et al., 2009) or muscle myofibers (von Maltzahn et al., 2011). Moreover, ectopic expression of Wnt7a does not induce the TOP-flash luciferase reporter (Molenaar et al., 1996) in C2C12 myoblasts (Kuroda et al., 2013). Different concentrations of Wnt7a were tested to exclude a dose dependent effect on β-catenin signaling, as measured by induction of TOP-flash activity in C2C12 cells. Canonical Wnt3a showed a dose dependent response (FIG. 4A); whereas, in contrast, increasing concentrations of Wnt7a did not increase TOP-flash reporter activity (FIG. 4B). These data indicated that Wnt7a acts through non-canonical Wnt signaling pathways in cells of the adult muscle lineage.

### Example 4

### Wnt7a Induced Cell Migration Involves Endocytosis

The effect of Wnt7a treatment on the subcellular distribution of Fzd7 was tested. Wnt7a stimulated primary myoblasts displayed a higher abundance of large intracellular aggregates and reduced peripheral Fzd7-YFP than controls (FIG. 4C). In addition, treatment with monodansylcadaverine (MDC), an inhibitor of clathrin-mediated endocytosis (Schlegel et al., 1982), prevented Wnt7a mediated migration in scratch assays (FIG. 4D). Furthermore, it was observed that myoblasts exposed to conditioned medium containing HA epitope tagged Wnt7a (Wnt7a-HA) quickly endocytosed this factor. After 3 h of incubation and several subsequent washing steps, endocytosed Wnt7a-HA was detectable for ≧72 h in intracellular structures in primary myoblasts (FIG. 4E) and C2C12 cells (FIG. 11E). No intracellular Wnt7a-HA could be observed in Fzd7−/− myoblasts exposed to the conditioned medium (FIG. 4E). These data showed that both Fzd7 and Wnt7a endocytosis are interdependent and requires activation of non-canonical signaling in myoblasts; that the endocytosis of Wnt7a was surprisingly fast and the protein appeared to be present in intracellular stores for prolonged periods; and that a short exposure to Wnt7a has sustained effects on myogenic cells through non-canonical Wnt signaling pathways.

### Example 5

### Wnt7a Treatment Facilitates the Migration of Primary Myoblasts In Vivo

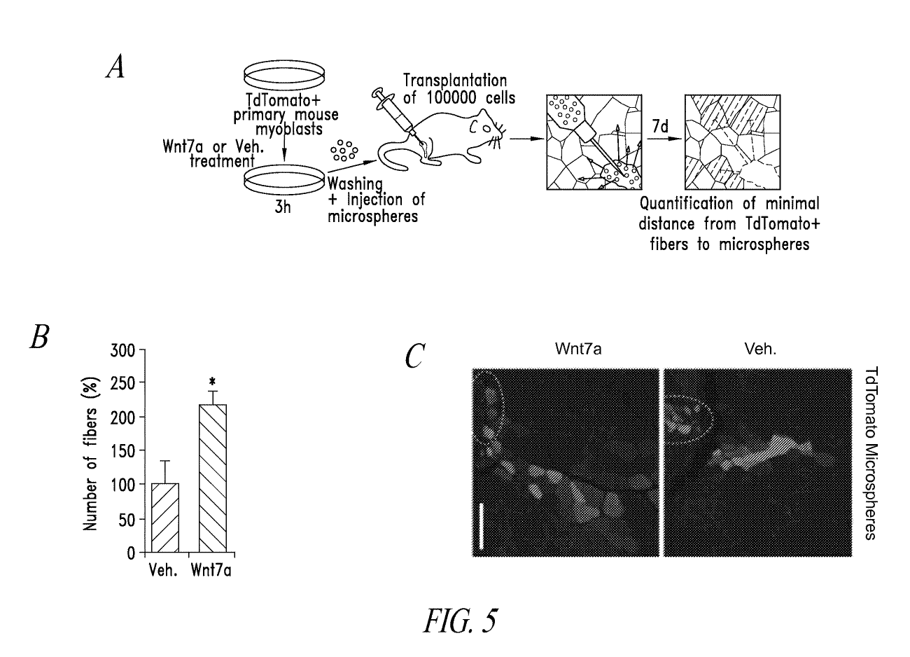

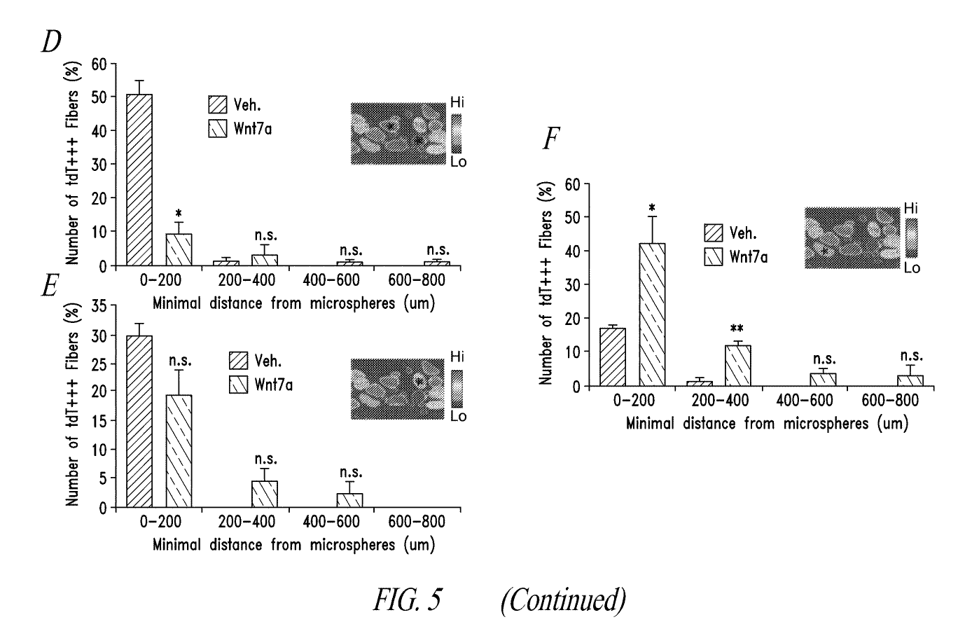

The duration of effects on cell migration were tested by loading intracellular stores with Wnt7a. tdTomato-expressing primary myoblasts, derived from tamoxifen treated Pax7-CreER; R26R-tdTomato mice (Yin et al., 2013a) were exposed to Wnt7a for 3 h; then the cells were washed and transplanted into the tibialis anterior (TA) muscle of immunosuppressed C57BL/6 mice (FIG. 5A). The myoblasts were co-injected with fluorescent microspheres to identify the injection site in subsequent analyses. 7 days later the mice were sacrificed and the behavior of the transplanted cells in the tissue was analyzed by scoring the number of tdTomato-expressing myofibers that were generated by fusion events with the donor cells.

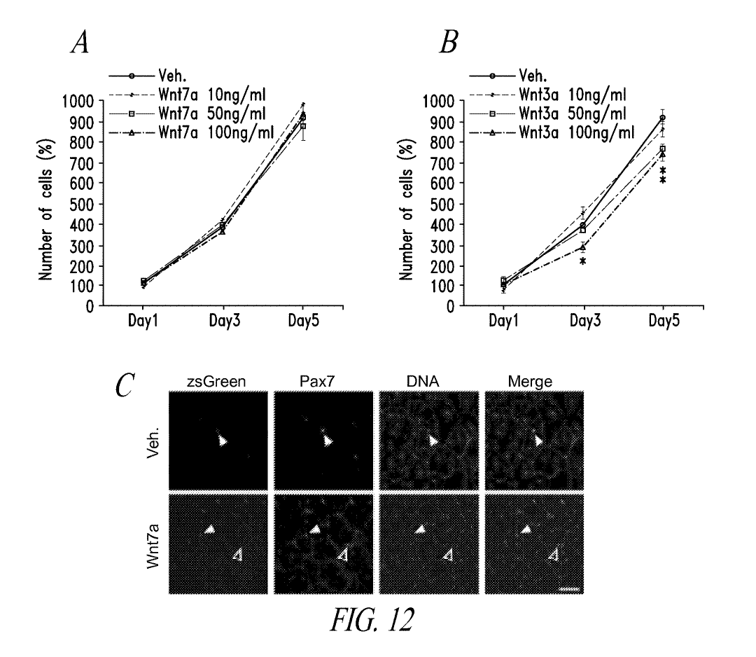

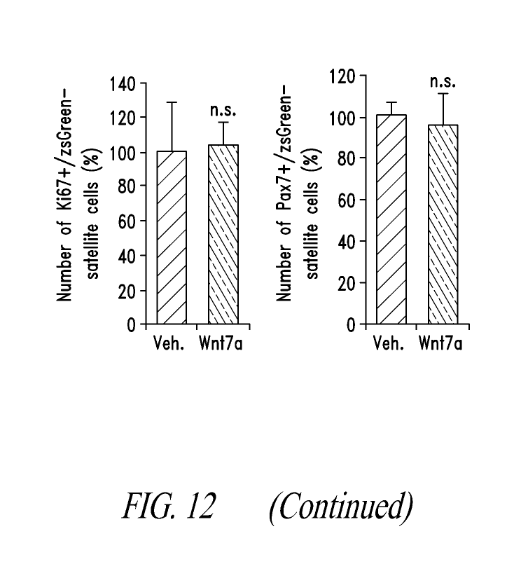

Wnt7a treatment increased the number of tdTomato-expressing myofibers by 119% (FIG. 5B). The proliferation of primary myoblasts following a 3 h exposure to various concentrations of Wnt7a was analyzed to control for the effect being due to changes in the cell cycle status of the transplanted cells. No change in the rate of proliferation was observed at any of the tested concentrations of Wnt7a over 5 days (FIG. 12A). By contrast, the induction of canonical signaling with higher concentrations of Wnt3a reduced the proliferation of primary myoblasts (FIG. 12B).

The increased number of tdTomato-expressing myofibers upon transplantation of Wnt7a treated myoblasts was not due to increased proliferation but was a consequence of enhanced dispersal. The observed increase in numbers of highly (tdT+++) tdTomato-expressing myofibers proximal to the injection site after transplantation of untreated cells (FIG. 5C-5E) relative to Wnt7a treated cells, provides strong evidence in support of this conclusion. The myofibers generated by fusion to Wnt7a-treated cells were weaker for tdTomato (tdT+), but higher in number and more spread out with respect to the injection site (FIGS. 5C and 5F). Thus, Wnt7a loading resulted in increased dispersal of the cells and lowered the chance for multiple fusion events to the same myofiber. Taken together, these data indicated that short exposure of myoblasts to Wnt7a dramatically enhances engraftment by promoting the tissue dispersion of the cells.

### Example 6

### Wnt7a Treatment Enhances the Outcome of Cell Therapy of Dystrophic Muscle

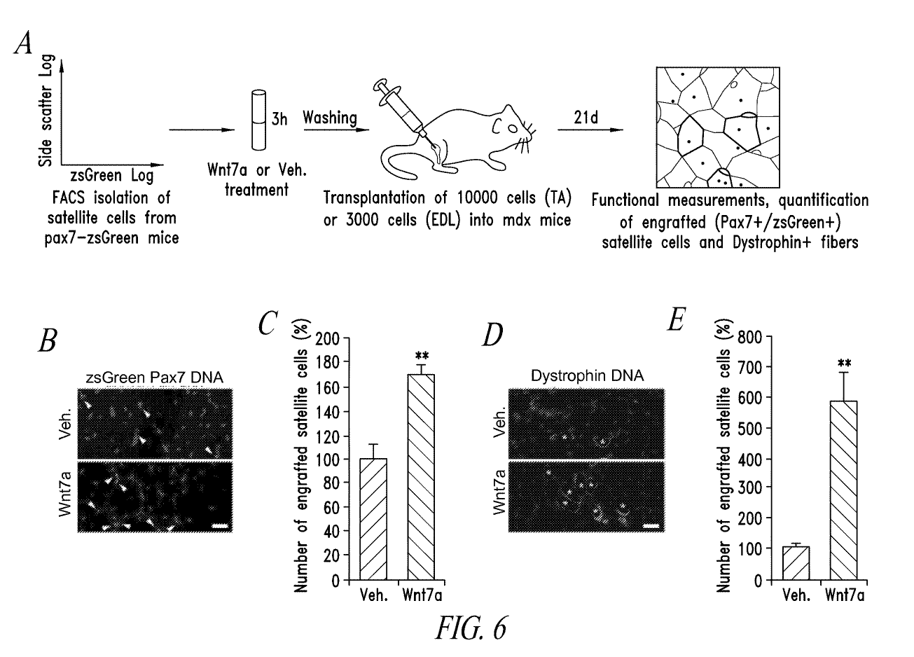

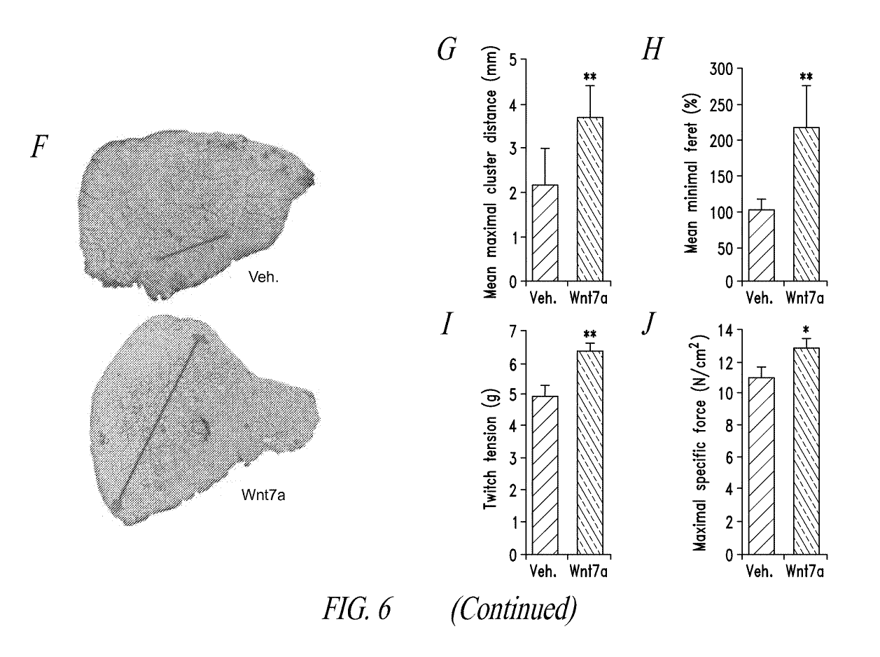

It is known that poor migration of myogenic cells upon intramuscular injection is a major hurdle for the development of cell based therapies for muscular dystrophy (Skuk et al., 2007). The present inventors tested whether satellite cells treated with Wnt7a ex vivo prior to transplantation would enhance engraftment. 10,000 satellite cells were isolated from Pax7-zsGreen mice by fluorescent activated cell sorting (Bosnakovski et al., 2008), treated with Wnt7a for 3 h; washed extensively; and transplanted into the TA of immunosuppressed dystrophin-deficient mdx mice that were injured with cardiotoxin 2 days before the procedure (FIG. 6A).

Wnt7a-treatment enhanced engraftment of zsGreen-expressing cells into the recipient muscles by 69% after 3 weeks, as determined by immunostaining for Pax7 and zsGreen in muscle sections (FIGS. 6B and 6C; FIG. 12C). Wnt7a-treatment also did not alter the proportion of zsGreen+ engrafted cells that express Ki67 (FIG. 14D) indicating that Wnt7a-treatment did not alter the rate of in vivo proliferation. In addition, the numbers of endogenous satellite cells was not altered (FIG. 14E), showing that resident satellite cells were not being stimulated by Wnt7a derived from the transplanted cells.

Under normal conditions, a portion of transplanted satellite cells will differentiate and fuse to muscle myofibers. In mdx mice, this process can be tracked by staining for restored dystrophin expression in muscle fibers. Using this assay, the number of myofibers expressing dystrophin increased on average by 486% following transplantation of Wnt7a-treated cells (FIGS. 6D and 6E). Similar to the stimulatory effect of Wnt7a on myogenic cell migration in vitro, clusters of dystrophin-expressing myofibers in muscles that were transplanted with Wnt7a-treated cells were highly dispersed and on average located maximally 3.7 mm apart. By contrast, transplantation of untreated cells gave rise to clusters of dystrophin-expressing myofibers that were on maximally 2.2 mm apart on average (FIGS. 6F and 6G). It was also observed that dystrophin-expressing myofibers arising after fusion with Wnt7a treated cells were hypertrophic by 116% (FIGS. 6D and 6H). Transplantation of 3,000 satellite cells treated ex vivo with Wnt7a into mdx extensor digitorum longus (EDL) muscles resulted in a striking 30% increase in twitch tension and to an 18% increase in maximal specific force generation when compared to the Veh. control (FIGS. 6I and 6J).

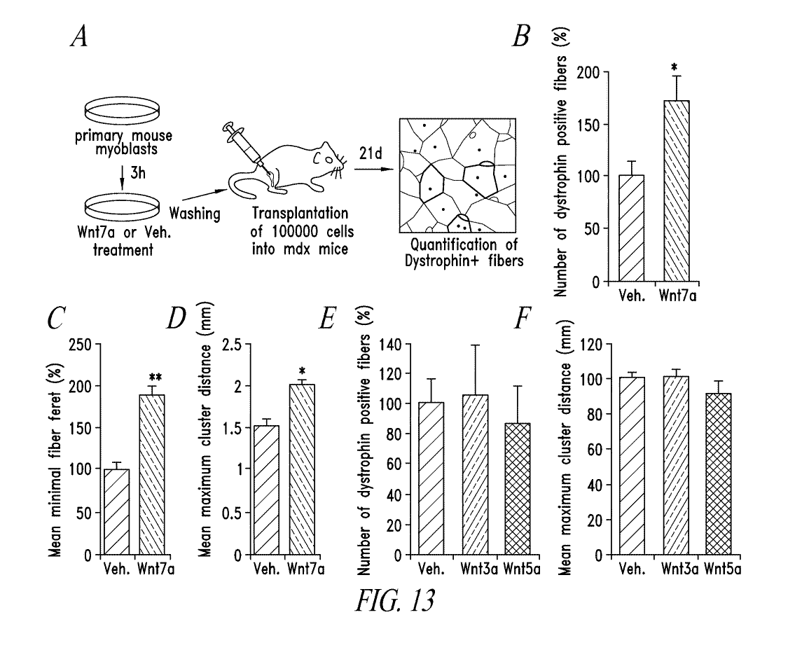

In a clinical setting, freshly isolated satellite cells are often not readily available. Therefore, the effect of 3 h Wnt7a loading on cultured primary mouse myoblasts that were subsequently transplanted into injured muscles of immunosuppressed mdx mice (FIG. S5 A) was examined. After transplantation of 100,000 cells, the number of dystrophin-expressing myofibers was increased by 72% in muscles that were injected with Wnt7a treated myoblasts (FIG. 13B). Moreover, dystrophin-expressing myofibers derived from Wnt7a-treated cells exhibited a 87% hypertrophy (FIG. 13C). In addition, the mean maximal cluster distance (dispersion) increased from 1.5 mm for Veh., to 2.0 mm for Wnt7a (FIG. 13D).

Improved engraftment efficiency is associated with Wnt7a. Neither the number of dystrophin-expressing myofibers (FIG. 13E) nor the mean maximal cluster distance (FIG. 13F) was changed by treatment with Wnt3a and Wnt5a. Taken together, these data indicated that Wnt7a treatment significantly enhanced the outcome of cell therapy of skeletal muscle. Transplantation of dystrophic muscle with Wnt7a-loaded cells led to an enhanced engraftment, enhanced tissue distribution of donor derived myofibers, and to improved force generation.

### Example 7

### Ex Vivo Wnt7a Treatment Stimulates Human Myoblast Transplantation

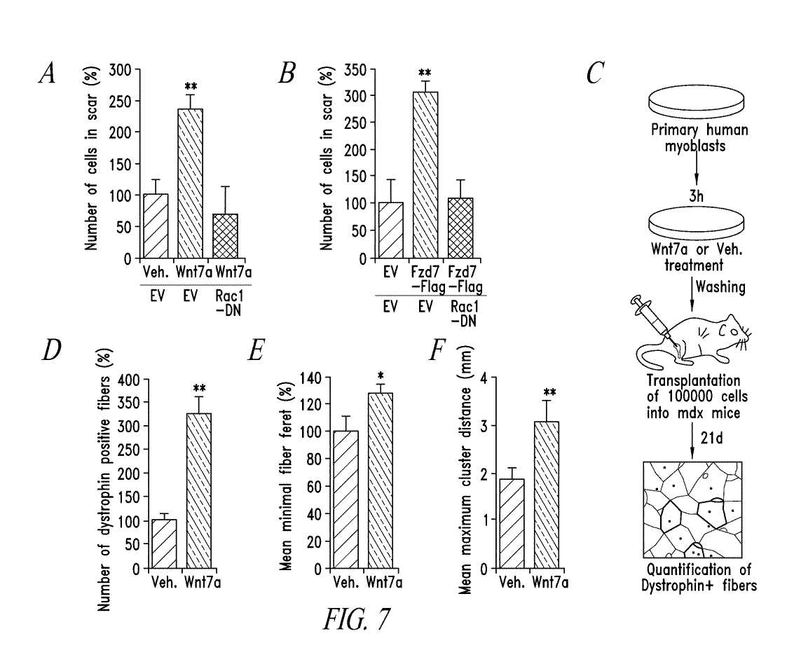

The translational relevance of Wnt7a treatment was examined in human primary myoblasts. Both Wnt7a treatment and Fzd7 overexpression significantly increased human myoblast migration, whereas Rac-DN prevented this effect (FIGS. 7A and 7B).

The effectiveness of Wnt7a on human myoblasts, in vivo, was tested using the same experimental paradigm that we previously used for mouse myoblasts (FIG. 7C). 100,000 human myoblasts were treated ex vivo with Wnt7a for 3 h, and transplanted into immunosuppressed mdx mice. Wnt7a treatment resulted in a 226% increase in the number of dystrophin-expressing myofibers in muscles of immunosuppressed mdx mice when compared to Veh. (FIG. 7D). Myofibers arising from fusion of mouse myofibers to Wnt7a-treated human myoblasts exhibited a 29% increase in fiber feret (FIG. 7E). Moreover, the mean maximal distance between dystrophin-expressing myofiber clusters increased from 1.8 mm under the Veh. condition to 3.0 mm for Wnt7a treatment (FIG. 7F). These data indicated that the Wnt7a induction of motility of myogenic cells is conserved in humans.

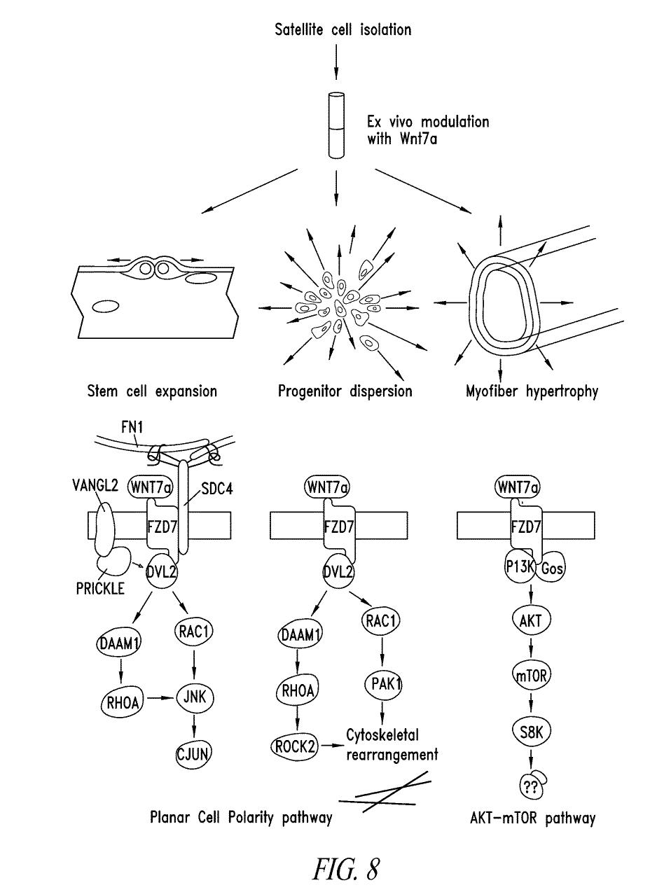

Taken together, the examples provided herein showed that ex vivo Wnt7a treatment of myogenic cells enhances the migration and tissue dispersion of both murine and human myogenic cells through Dvl2 and the small GTPases. This effect was most pronounced in Pax7⁺/Myf5⁻ satellite cells, but also prevalent in Pax7⁺/Myf5⁺ satellite cells, indicating that committed myogenic progenitors readily activate non-canonical signaling in response to Wnt7a. Thus, Wnt7a has several therapeutically attractive properties that modulate muscle regeneration at multiple levels including the stimulation of motility and engraftment (FIG. 8).

### Example 8

### Experimental Summary

Strategies to treat progressive degenerative muscle diseases include the genetic correction of affected muscle fibers and the restoration of tissue regenerative capacity. The ability of myogenic cells to add their nuclei to the syncytial muscle fibers through fusion makes them an ideal candidate for cell therapy of genetic diseases that affect myofiber stability or function. Moreover, life-long muscle hypertrophy as a consequence of transplanting a small number of satellite cells raise hope for the use of these cells as a therapeutic option for conditions such as burns, cancer cachexia and sarcopenia that are accompanied by uncontrolled catabolism of muscle protein.

In humans, satellite cells account for less than 5% of total myofiber nuclei (Lindstrom and Thornell, 2009). In addition, the amount of tissue that can be obtained from a muscle biopsy is relatively small. Therefore, the availability of sufficient donor cells is a major obstacle for the effective therapy of affected muscle groups. Compounding this problem, in vitro expansion of satellite cells profoundly impairs their stem cell character and decreases the efficiency of engraftment several fold.

The effects of Wnt7a on transplanted satellite cells were shown to be more profound when compared to human and mouse myoblasts (Compare FIGS. 6 and 7; and FIG. 13). Nevertheless, Wnt7a was also beneficial for the outcome of myoblast transplantation. Following ex vivo Wnt7a treatment, human myoblasts generated >200% dystrophin-expressing myofibers that were also distributed more evenly throughout the tissue (FIGS. 7D and 7F). Thus, the dose of cells required for therapy may be up to 3-fold more effective if they are treated with Wnt7a before transplantation.

The transplantation of ex vivo Wnt7a-loaded cells into dystrophic muscles resulted in a significant increase in both the twitch tension and the maximal specific force, when compared to Veh. (FIGS. 6I and 6J). The enhanced twitch tension was a direct measure of muscle strength, and was due, in part, to myofiber hypertrophy induced by fusion of Wnt7a-loaded cells (FIGS. 6H and 6I). By contrast, the improved contraction quality under tetanic stimulation likely reflects the higher number of more evenly distributed dystrophin-expressing myofibers in the Wnt7a condition (FIGS. 6E and 6J).

The experiments conducted herein demonstrate that the enhanced tissue dispersion of Wnt7a stimulated cells was due to directed migration (FIGS. 2H and 2I) and did not involve induction of proliferation (FIG. 12A). In addition, the effect of Wnt7a on the migration of myogenic progenitors was β-catenin independent (FIG. 10B and FIG. 4B) and involved effectors of non-canonical Wnt signaling such as Dvl2 and small GTPases (FIG. 3 and FIG. 11D).

An enhanced ability of cells to migrate away from the injection site to prevent or escape necrotic cell death, contributed to the increased engraftment of Wnt7a treated cells in muscle tissue. Both Fzd7 and its downstream effector Rac1 showed a characteristic subcellular localization within vesicles in the cytoplasm and accumulated in the periphery of the cell (FIGS. 1B and 1C; and FIG. 3A). Fzd7 colocalized with Rac1 in the cellular periphery, while intracellular vesicle associated Fzd7 did not co-localize with Rac1 (FIG. 3A) and Fzd7-YFP was dynamically rearranged at the leading edge of migrating cells (FIG. 1C). Thus, Fzd7 locally activated Rac1 to facilitate directed migration. In addition, the localization of Fzd7 at the leading edge of migrating cells may have allowed the cells to respond to gradients of Wnt.

Endocytosis of Fzd receptors upon Wnt stimulation plays a role in non-canonical Wnt signaling. In the case of Fzd2, clathrin mediated endocytosis is a prerequisite for the activation of Rac1 (Sato et al., 2010). The data disclosed herein showed that clathrin dependent endocytosis also plays a role in the induction of Wnt7a/Fzd7 signaling (FIG. 4D) and that large fraction of Fzd7 localized to intracellular vesicles that seemed to be attached to the tubulin cytoskeleton (FIG. 1B). Wnt7a treatment led to partitioning of Fzd7 away from the cellular periphery into these intracellular structures (FIG. 4 C). It was also observed that myogenic cells readily endocytosed Wnt7a in a Fzd7-dependent manner after only a few hours of exposure (FIG. 4E and FIG. 11E). Unexpectedly, intracellular Wnt7a could be detected in the cells for periods longer than 72 h after internalization. Muscle fibers fused to such Wnt7a-loaded cells became hypertrophic (FIG. 4H; FIG. 12F; and FIG. 5E). Thus, Wnt7a can be released from intracellular storage to induce the signaling events leading to the hypertrophic response.

Due to their lipid modifications and other unique biochemical properties, Wnt proteins are notoriously difficult to purify (Willert and Nusse, 2012). This undoubtedly complicates their use as a therapeutic. However, exposure of cells to Wnt7a in the nanomolar range was sufficient to produce extremely long lasting effects. Thus, sufficient production of pharmaceutical grade Wnt7a for standardized therapies can be achieved.

In summary, the data demonstrate that Wnt7a loading is a highly efficient means to modulate human myogenic cells before transplantation. Binding of Wnt7a to Fzd7 leads to an activation of non-canonical Wnt signaling resulting in directed cell migration and enhanced engraftment. Moreover, Wnt7a-loaded cells are remarkably effective in increasing the growth and strength of dystrophic muscle.

### Example 9

### Materials and Methods

### Mice and Animal Care

Pax7-CreER; R26R-tdTomato, Pax7-zsGreen and Fzd7 knockout mice were described previously (Bosnakovski et al., 2008; Yin et al., 2013a; Yu et al., 2012). mdx mice were obtained from Jackson Laboratories. All experiments were performed in accordance with University of Ottawa guidelines for animal handling and care.

### Transplantation

Transplantation of zsGreen-expressing satellite cells or myoblasts into mdx mice was performed as previously described (Bentzinger et al., 2013c). Before transplantation the cells were treated for 3 h with Wnt7a, Wnt5a, or Wnt3a (R&D Systems) at a concentration of 50 ng/mL for freshly isolated satellite cells or with 100 ng/mL for cultured cells. Myoblasts expressing tdTomato were transplanted into uninjured muscles of C57BL/6 mice that were implanted with an osmotic pump (Alzet) delivering FK506 (Sigma) at 2.5 mg/kg/day 3 days prior to the transplantations. In some experiments, the injection site was marked with blue fluorescent microspheres (2 μm, Life Technologies) that were added at a concentration of 1:100 (vol/vol) to the cell suspension. 10 μl of the cell-microsphere suspension was injected into the TA of each mouse using a Hamilton syringe.

### Muscle Force Measurements

Extensor digitorum longus (EDL) muscles were isolated three weeks following transplantation and attached to an electrode and a force sensor (Aurora Scientific), and incubated at 25° C. in buffered physiological saline (Krebs-Ringer) supplemented with glucose and oxygen. Twitch force was determined following a single electrical stimulus, while maximal force was generated by electrical stimulation at 100 Hz for 500 ms. Thereafter, muscle length and weight were measured and specific muscle force was calculated ((maximal force×optimal fiber length×muscle density)/muscle mass).

### Live Imaging

Single cell imaging of cells transfected with Fzd7-YFP or YFP was performed on an LSM710 confocal microscope under 5% CO₂ at 37° C. in phenol-red free DMEM (Life technologies) containing 10% FBS. Imaging of satellite cells on fibers was performed as described (Siegel et al., 2009). For live imaging of satellite cells on myofibers, Wnt7a was used at a concentration of 1.5 μg/mL. Satellite cell time-lapse microscopy was performed using a Leica DMI 5100 inverted microscope and MetaMorph 7.6.1 Software (Molecular Devices) with time points being acquired every 7 minutes in a stagetop incubator (LiveCell Imaging). ImageJ analyzed the time-lapse data to calculate the velocity, defined as distance (um)/time (hr), between time points and the average velocity of each cell was calculated. The maximum velocity is defined as the highest velocity obtained by a single cell during the tracking period.

### Primary Myoblast Isolation and Culture

For myoblast culture, satellite cells were FACS purified as described (Bentzinger et al., 2013c) and plated on Collagen-coated dishes (BD Biosciences) in Ham's F10 medium supplemented with 20% FBS and 5 ng/mL of basic FGF (Millipore). C2C12 cells and human myoblasts were cultured as previously described (von Maltzahn et al., 2012). For in vitro morphology quantifications, Wnt7a was used at a concentration of 50 ng/mL.

### Scratch Assays

Following plasmid transfection or application of Wnts (R&D Systems), the cells were treated with Mitomycin-C for 2 h (50 μg/mL). Subsequently, the monolayer of cells was scraped in a straight line. The plates were then extensively washed with culture medium and incubated for 24 h before analysis. Analysis was performed using DAPI staining after matching the reference points and enumeration of DAPI-stained nuclei in the scar was performed. Unless otherwise indicated, Wnt7a and Wnt3a were used at a concentration of 100 ng/mL in scratch assays. Monodansylcadaverine (MDC, 30432, Sigma) was used in scratch assays at a concentration of 50 μm.

### Western Blotting and Immunoprecipitation

For co-immunoprecipitation (CoIP) experiments and the Rac1 activation assay, satellite cell-derived primary myoblasts were infected with retroviruses generated from empty pHAN or from pHAN-Wnt7a-HA (Kuroda et al., 2013). Dvl2 knockdown was performed with an ON-TARGETplus siRNA SMARTpool (L-040921-01, Thermo Fisher Scientific) (Wu et al., 2008) using Lipofectamine RNAiMAX (Life Technologies) according to the manufacturer's instructions. As a control for the knockdown, a non-silencing siRNA SMARTpool was used (D-001810-10, Thermo Fisher Scientific). Cell extracts were obtained through RIPA buffer lysis in the presence of a protease inhibitor cocktail (Nacalai). For CoIP of Rac1 with Dvl2, rabbit anti-Dvl2 antibody was coupled to Protein A Dynabeads (Life Technologies). Denaturing SDS-PAGE was performed using standard techniques. Rac1 activation assay was performed according to the manufacturer's instructions (Pierce).

### Immunostaining and Antibodies

Muscles frozen in liquid nitrogen were cut into 12 μm cross-sections. Cross-sections and cells were either fixed with ethanol (100%) or 2% PFA for 5 min, permeabilized with 0.1% Triton/0.1 M glycine/PBS for 10 min, blocked with 5% horse serum in PBS for several hours, and incubated with specific primary antibody in blocking buffer overnight at 4° C. Samples were subsequently washed with PBS and stained with appropriate fluorescently labeled secondary antibodies for 1 h at room temperature. After washing with PBS, samples were mounted with Permafluor (Fisher). Antibodies were as follows: chicken anti-GFP (Abcam, ab13970), mouse anti-Pax7 (DSHB), mouse anti-Dystrophin (7A10, DSHB), mouse anti-Rac1 (05-389, Millipore), mouse anti-Tubulin (Sigma, T9026), rabbit anti-Dvl2 (3224, Cell Signaling), rabbit anti-zsGreen (Clontech, 632474), rabbit anti-Laminin (Sigma, L9393), rabbit anti-HA (Millipore, 07-221).

### Real-Time PCR

Total RNA was isolated (NucleoSpin RNA II, Macherey-Nagel). Reverse transcription was carried out using a mixture of oligodT and random hexamer primers (iScript cDNA Synthesis Kit, Bio-Rad). Sybr Green, real-time PCR analysis (iQ SYBR Supermix, Bio-Rad) was performed using Mx300P real time thermocycler (Stratagene). The following primers were used: Dvl2 sense: ACGACGATGCTGTACGAGTG (SEQ ID NO: 86), Dvl2 anti-sense: CGAGGGAGGGTGAAGTAGG (SEQ ID NO: 87), Fzd7 sense: GCTTCCTAGGTGAGCGTGAC (SEQ ID NO: 88), Fzd7 anti-sense: AACCCGACAGGAAGATGATG (SEQ ID NO: 89).

### Plasmids and Transfection

The UBC-Fzd7-Flag (Fzd7-Flag), CMV-Fzd7-EYFP (Fzd7-YFP), CMV-Fzd3-EYFP (Fzd3-YFP), CMV-EYFP (YFP), CMV-EGFP-Rac1-wt (GFP-Rac1, 12980, Addgene), CMV-EGFP-Rac1-T17N (Rac1-DN, 12982, Addgene), CMV-EGFP-RhoA-T19N (RhoA-DN, 12967, Addgene) and CMV-EGFP-Cdc42-T17N (Cdc42-DN, 12976, Addgene) constructs have been described previously (Bentzinger et al., 2013a; Subauste et al., 2000; von Maltzahn et al., 2011). The Fzd7-tdtomato plasmid was generated by replacing the C-terminal YFP in Fzd7-YFP with tdTomato. For TOP-flash and FOP-flash the TCF reporter plasmid kit (17-285, Millipore) was used with the dual-luciferase reporter assay system (E1960, Promega). For normalization pGL4.74[hRluc/TK] (E6921, Promega) was cotransfected. For TOP-flash and FOP-flash assays the cells were transfected with GenJet lipofection reagent (SL100499, SignaGen). Otherwise, Lipofectamine 2000 (11668019, Life Technologies) was used for transfections.

### Statistical Analysis

Densitometry of gray values from western blots was performed with ImageJ software. Compiled data are expressed as the mean±SEM. Experiments were done with a minimum of three biological replicates. For statistical comparison of two conditions, the Student's t test was used. The level of significance is indicated as follows: ***p<0.001, **p<0.01, *p<0.05.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method of increasing engraftment of a cell comprising:
(a) contacting the cell with or introducing into the cell one or more non-canonical Wnt signaling activators in vitro, for a time sufficient to increase non-canonical Wnt signaling in the cell; and
(b) administering the contacted cell to a subject in need thereof;
wherein the administered cell has an increased engraftment potential compared to a non-contacted cell.

2. The method of claim 1, wherein the cell is a stem cell or progenitor cell.

3. (canceled)

4. The method of claim 1, wherein the cell is a myogenic cell.

5. The method of claim 1, wherein the cell is a muscle satellite stem cell.

6.-11. (canceled)

12. The method of claim 1, wherein the cell is not a hematopoietic cell.

13. The method of claim 1, wherein the cell is genetically modified.

14. The method of claim 1, wherein the non-canonical Wnt signaling activator is selected from the group consisting of a small molecule, a nucleic acid, a polypeptide, and suitable combinations thereof.

15. The method of claim 14, wherein the polypeptide comprises a non-canonical Wnt polypeptide or biologically active modified non-canonical Wnt polypeptide.

16. The method of claim 15, wherein the biologically active modified non-canonical Wnt polypeptide comprises one or more N-terminal or C-terminal truncations, or one or more amino acid additions, deletions, or substitutions.

17. (canceled)

18. The method of claim 16, wherein the lipidation of the biologically active modified non-canonical Wnt polypeptide is reduced.

19. The method of claim 15, wherein the non-canonical Wnt polypeptide comprises a Wnt7a polypeptide.

20. The method of claim 1, wherein the polypeptide is a Fzd7 polypeptide or modified Fzd7 polypeptide.

21. The method of claim 1, wherein engraftment potential is increased by an increase in cell motility, cell migration, myofusion or a combination thereof.

22. A myogenic cell-based gene therapy comprising:
(a) a myogenic cell comprising an exogenous polynucleotide;
(b) contacting the myogenic cell in vitro with at least one non-canonical Wnt signaling activator for a time sufficient to increase non-canonical Wnt signaling in the cell; and
(c) administering the contacted myogenic cell to a subject in need of gene therapy;
wherein fusion of the myogenic cell with a myofiber in the subject delivers the polynucleotide to the subject.

23.-38. (canceled)

39. The myogenic cell-based gene therapy of claim 22, wherein the subject has a disorder selected from the group consisting of: cachexia, cancer, AIDS, muscular attenuation, muscle atrophy, muscle trauma, muscle injury, surgery, disuse atrophy, or a muscle degenerative disease.

40. The myogenic cell-based gene therapy of claim 22, wherein the subject has a disorder selected from the group consisting of: Duchenne muscular dystrophy (DMD), Becker muscular dystrophy (BMD), Emery-Dreifuss muscular dystrophy, Landouzy-Dejerine muscular dystrophy, facioscapulohumeral muscular dystrophy (FSH), Limb-Girdle muscular dystrophies, von Graefe-Fuchs muscular dystrophy, oculopharyngeal muscular dystrophy (OPMD), Myotonic dystrophy (Steinert's disease) and congenital muscular dystrophies.

41.-60. (canceled)

61. A method of increasing cell graft efficacy comprising:
(a) contacting a cell graft in vitro with a non-canonical Wnt signaling activator for a time sufficient to increase the engraftment potential of the cell graft; and
(b) administering the contacted cell graft to a subject in need thereof;
wherein the administered cell graft has increased engraftment compared to a non-contacted cell graft.

62.-83. (canceled)

84. A culture comprising:
(a) a population of myogenic cells; and
(b) an exogenous non-canonical Wnt signaling pathway activator in an amount sufficient to increase the engraftment potential of the population of cells.

85. The culture of claim 84, wherein the population of myogenic cells comprises Pax7⁺/Myf5⁻/MyoD⁻ cells, Pax7⁺/Myf5⁺/MyoD⁻ cells, and/or Pax7⁺/Myf5⁺/MyoD⁺ cells.

86.-99. (canceled)

100. A method of preventing, ameliorating, or treating a muscle disorder in a mammal in need thereof comprising:
a) contacting a myogenic cell comprising a polynucleotide encoding a polypeptide-of-interest with one or more non-canonical Wnt signaling activators, in vitro; and
b) administering the contacted myogenic cell to the mammal.

101.-119. (canceled)
